(19) United States
(12) Patent Application Publication
(10) Pub. No.: US 2003/0188343 A1
(43) Pub. Date: Oct. 2, 2003

(54) Identification of genes associated with growth in plants

(75) Inventors: Benjamin A. Bowen, Berkeley, CA (US); Christian D. Haudenschild, Oakland, CA (US); Edward S. Buckler IV, Raleigh, NC (US)

Correspondence Address: QUINE INTELLECTUAL PROPERTY LAW GROUP, P.C., P O BOX 458, ALAMEDA, CA, 94501, US

(73) Assignee: Lynx Therapeutics, Inc., Hayward, CA (US)

(21) Appl. No.: 10/338,777

(22) Filed: Jan. 7, 2003

Related U.S. Application Data
(60) Provisional application No. 60/347,288, filed on Jan. 9, 2002.

(51) Int. Cl. A01H 1/00; C12N 5/04; C12Q 1/68; C07H 21/04; C12N 15/82

(52) U.S. Cl. 800/287; 435/6; 435/419; 435/468; 536/23.6

(57) ABSTRACT

Genes, nucleic acids and polypeptides associated with growth traits in plants are provided. Related probes, antibodies, marker sets, and arrays are provided as well as methods for predicting plant growth traits.

### CROSS-REFERENCE TO RELATED APPLICATIONS

This application claims priority to and benefit of a prior U.S. Provisional Application No. 60/347,288, Identification of Genes Associated with Growth in Plants, by Benjamin A Bowen, et al., filed Jan. 9, 2002. The full disclosure of the prior application is incorporated herein by reference.

### STATEMENT AS TO RIGHTS TO INVENTIONS MADE UNDER FEDERALLY SPONSORED RESEARCH AND DEVELOPMENT

The work of Edward S. Buckler IV was sponsored by USDA CRIS 6645-21000-022-00D.

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

This invention is in the field of genes which control growth traits in plants. The present invention relates, e.g., to the identification of candidate genes associated with growth in plants, polypeptides encoded by these genes, related probes, marker sets, methods for predicting the presence of growth traits in plants, and the like.

### BACKGROUND OF THE INVENTION

Improvement of plant crops has generally proceeded incrementally through the intentional and/or incidental selection of individual plants with desired traits for cultivation. Crossing of unique individuals can result in vigorous individual hybrid plants with desirable characteristics. These established methods of hybrid generation and selection have provided mankind with vastly improved crop plants, but continued improvement by these methods is slow and unpredictable.

Plant growth traits are among the most important crop characteristics in commercial agriculture. The green revolution has increased plant growth rates with fertilizers and inhibited plant (weed) growth through herbicide application, providing significant improvements in crop yields to feed the world population since at least the 1960s. However, marginal improvements in green revolution technologies are tapering off and new approaches are needed to increase the productivity of agriculture.

Agricultural biotechnology can provide a directed approach to enhancing the quality and quantity of crops. Identification of genes associated with a desired plant characteristic, or trait, can be the first step to control of the trait. Gene recombination technologies can be employed to incorporate the identified genes into expression systems which can modulate display of a trait, screen for plants having a trait, and/or screen for additional genes associated with the trait. Plant growth traits are of special significance in agriculture, and identification of genes controlling plant growth is critical to providing food for the growing world population. Thus, identification and characterization of gene(s) controlling plant characteristics is of great interest, and will be of significant scientific and commercial importance.

The present invention relates to the identification of genes associated with plant growth traits. Polypeptides encoded by these genes, as well as related probes, marker sets, and methods for predicting growth traits in plants, as well as other features, will become apparent upon review of the following materials.

### SUMMARY OF THE INVENTION

The present invention relates to a set of polynucleotide sequences which control growth traits in plants, exemplified by, e.g., SEQ ID NO: 1 through SEQ ID NO: 30 and, e.g., a set of polypeptide sequences which control growth traits in plants, exemplified by, e.g., SEQ ID NO: 31 through SEQ ID NO: 60.

In a first aspect, the invention relates to compositions including one or more nucleic acid expression vectors which include the polynucleotide sequences of the invention. For example, such expression vectors include nucleic acids including at least one polynucleotide sequence selected from SEQ ID NOs: 1-30. Similarly, sequences that hybridize under stringent hybridization conditions, or that are at least about 70%, (or at least about 75%, about 80%, about 85%, about 90%, about 95%, about 97%, about 98%, or at least about 99%) identical to one or more of SEQ ID NO: 1-30 can be included in the expression vectors of the invention. In addition, expression vectors, including polynucleotide sequences that encode a polypeptide sequence selected from among SEQ ID NO: 31-SEQ ID NO: 60, or conservative variations thereof, are compositions of the invention. Likewise, expression vectors incorporating nucleic acids with subsequences of at least 10 contiguous nucleotides of, e.g., SEQ ID NOs: 1-30 (or at least 12, 14, 16, or 17 or more contiguous nucleotides of one of the designated sequences) are included among the compositions of the invention. The polynucleotide sequences of the invention also include polynucleotide sequences complementary to any one of the above polynucleotide sequences described above. In some embodiments, the expression vector includes a promoter operably linked to one or more of the nucleic acids described above. Such expression vectors can encode expression products such as sense or antisense RNAs, or polypeptides.

Polypeptides having an amino acid sequence selected from the group consisting of SEQ ID NO: 31 to SEQ ID NO: 60, and conservative variants thereof, are also a feature of the invention, as are polypeptides encoded by a polynucleotide sequence of the invention (e.g., SEQ ID NO: 1-SEQ ID NO: 30, sequences that hybridize under stringent conditions to any one of SEQ ID NO: 1-SEQ ID NO: 30, sequences that are at least about 70% identical to any one of SEQ ID NO: 1-SEQ ID NO: 30, sequences that encode a polypeptide or conservative variations any such sequences, or subsequences thereof). Polypeptides (and oligopeptides and peptides) including amino acid subsequences of SEQ ID NO: 31 through SEQ ID NO: 60 are also a feature of the invention. For example, fusion proteins including a polypeptide of SEQ ID NO: 31 through SEQ ID NO: 60, or a subsequence, e.g., an antigenic subsequence, thereof are included in the polypeptides of the invention. Likewise, proteins having a sequence selected from SEQ ID NO: 31 to SEQ ID NO: 60, and homologous or variant polypeptides, and a peptide or polypeptide tag, such as a reporter peptide or polypeptide, localization signal or sequence, or antigenic epitope, are included among the polypeptides of the invention.

Cells comprising an expression vector, and/or expressing a polypeptide as described above, are also a feature of the invention. In certain embodiments, the expressed polypeptide can be encoded by an exogenous polynucleotide, e.g., an expression vector. Such expression vectors typically include a polynucleotide sequence encoding the polypeptide of interest operably linked to, and under the transcriptional regulation of, a constitutive or inducible promoter. In other embodiments, the polypeptide is encoded by an endogenous polynucleotide sequence activated by an exogenous promoter and/or enhancer.

Antibodies specific for the polypeptides of the invention, e.g., SEQ ID NO: 31-SEQ ID NO: 60, and conservatively modified variants, etc., are also a feature of the invention. Such specific antibodies can be either derived from a polyclonal antiserum or can be monoclonal antibodies. For example, such antibodies are specific for an epitope including or derived from a subsequence of one of SEQ ID NO: 31-SEQ ID NO: 60.

Another aspect of the invention provides labeled nucleic acid or polypeptide probes. For example, nucleic acid probes of the invention include DNA or RNA molecules incorporating a polynucleotide sequence of the invention e.g., selected from SEQ ID NO: 1-SEQ ID NO: 30, sequences that hybridize under stringent conditions to any one of SEQ ID NO: 1-SEQ ID NO: 30, sequences that are at least about 70% identical to any one of SEQ ID NO: 1-SEQ ID NO: 30, sequences that encode a polypeptide selected from SEQ ID NO: 1-SEQ ID NO: 30, sequences complementary to any such sequences, or a subsequence thereof including at least 10 contiguous nucleotides. Optionally, the subsequences include at least 12 contiguous nucleotides of one of, e.g., SEQ ID NOs: 1-30. Often such subsequences include at least 14 contiguous nucleotides, typically at least 16 contiguous nucleotides, and usually at least 17 or more contiguous nucleotides, e.g., of SEQ ID NO: 1 to SEQ ID NO: 30. These nucleic acid probes can be, e.g., synthetic oligonucleotides and probes, cDNA molecules, amplification products (e.g., produced by PCR or LCR), transcripts, or restriction fragments. In other embodiments, the labeled probes are polypeptides, such as polypeptides with amino acid sequences corresponding to SEQ ID NOs: 31-60, or subsequences thereof (e.g., peptide subsequence comprising at least six amino acids), including peptide subsequences. Antibodies specific for such polypeptides or peptides are also a feature of the invention (as are polypeptides which bind to such antibodies). For example, a polypeptide probe can be a fusion protein, or a polypeptide with an epitope tag. A peptide probe can be an antigenic peptide derived from one of SEQ ID NO: 31 through SEQ ID NO: 60.

The label of the nucleic acid, polypeptide or antibody probe can be any of a variety of detectable moieties including isotopic, fluorescent, fluorogenic, or colorimetric labels.

In another aspect, the invention relates to a marker set, e.g., for predicting at least one growth trait of a plant cell. Such marker sets can include a plurality of members, where the members comprise nucleic acids, polypeptides, and/or peptides, and/or antibodies. Marker sets can include two or more of one type of member, or optionally can include one or more of two or more different types of members. For example, marker sets can include a plurality of nucleic acids including one or more polynucleotide sequence selected from SEQ ID NO: 1 to SEQ ID NO: 30, or SEQ ID NO: 61 to SEQ ID NO: 403, or conservative modifications thereof; polynucleotide sequences that hybridize under stringent hybridization conditions, or that are at least about 70%, (or at least about 75%, 80%, 85%, 90%, 95%, 97%, 98%, or at least about 99%) identical to one or more of SEQ ID NOs: 1-30; sequences complementary to any such sequences or subsequences thereof including at least 10 contiguous nucleotides of, e.g., SEQ ID NOs: 1-30 (or at least 12, 14, 16, 17 or more contiguous nucleotides of one of the designated sequences).

In one embodiment, the marker set includes a plurality of oligonucleotides, such as synthetic oligonucleotides. In other embodiments, the marker set includes expression products, amplification products, nucleic acid probes, or the like. The marker set of the invention can also include multiple nucleic acids selected from among different molecular classifications, e.g., oligonucleotides, expression products (such as cDNAs), amplification products, restriction fragments, etc. In one embodiment, the marker set is made up of nucleic acids including polynucleotide sequences corresponding to each of SEQ ID NO: 1 through SEQ ID NO: 30, or a subsequence selected from each of SEQ ID NO: 1 through SEQ ID NO: 30, or their compliments. In one embodiment, the marker set is made up of a plurality or a majority of members that together comprise a plurality, majority, or all of sequences or subsequences selected from a plurality, a majority or each nucleic acid represented by SEQ ID NO: 61-SEQ ID NO: 403, or their compliments.

Markers of the invention can also be polypeptides, e.g., polypeptides encoded by SEQ ID NO: 31-SEQ ID NO: 60, or polypeptide or peptide subsequences thereof. Typically, a peptide subsequence comprises, e.g., at least about 6 contiguous amino acids, 10 contiguous amino acids or more, often at least about 15 contiguous amino acids, and frequently at least about 20 contiguous amino acids of, e.g., one of SEQ ID NOs: 31-60.

Markers of the invention can also be antibodies, e.g., monoclonal or polyclonal antibodies, or anti-sera specific for an epitope derived from a polypeptide of the invention, e.g., one or more of SEQ ID NO: 31 through SEQ ID NO: 60.

In certain useful embodiments, the marker set is logically or physically arrayed. For example, the members of the marker set, whether nucleic acid, polypeptide, peptide or antibody, or a combination thereof, can be physically arrayed in a solid phase or liquid phase array, such as a bead (or microbead) array. Arrays, including a plurality of SEQ ID NO: 1 to SEQ ID NO: 30, SEQ ID NO: 31-SEQ ID NO: 60, SEQ ID NO: 61-SEQ ID NO: 403, or antibodies specific therefor, are also a feature of the invention. In some embodiments, the arrays include members corresponding to a majority of SEQ ID NO: 1 to SEQ ID NO: 30, SEQ ID NO: 61-SEQ ID NO: 403, SEQ ID NO: 31 to SEQ ID NO: 60, or antibodies specific therefor. In one embodiment, the array includes members corresponding to each of SEQ ID NO: 1 to SEQ ID NO: 30, SEQ ID NO: 31 to SEQ ID NO: 60, or antibodies specific therefor. In an embodiment, the marker set is comprised of at least 10 contiguous nucleotides of each of SEQ ID NO: 61-SEQ ID NO: 403, at least 10 contiguous nucleotides of a plurality of SEQ ID NO: 61-SEQ ID NO: 403, at least 10 contiguous nucleotides of a majority of SEQ ID NO: 61-SEQ ID NO: 403, or complimentary sequences thereof. In an embodiment, the marker set is a mixed marker set including members that are selected from nucleic acids, polypeptides or peptides, and antibodies.

In one embodiment, the marker set of the invention is used to predict at least one growth trait of a plant cell by hybridizing one or more nucleic acids of the marker set to a DNA or RNA sample from a cell or tissue, and detecting at least one polymorphic polynucleotide or differentially expressed expression product in the sample. In another related embodiment, differentially expressed expression products are detected using an array, e.g., an antibody array.

Another aspect of the invention provides methods for modulating a plant growth trait. The methods of the invention for modulating plant growth in a cell or tissue optionally include modulating expression or activity of at least one polypeptide encoded by a nucleic acid with a polynucleotide sequence selected from SEQ ID) NO: 1 to SEQ ID NO: 30, or conservative modifications thereof; a polynucleotide sequence encoding a polypeptide sequence selected from SEQ ID NO: 31 to SEQ ID NO: 60; a polynucleotide sequence that hybridizes under stringent hybridization conditions, or that is at least 70%, (or at least 75%, 80%, 85%, 90%, 95%, 97%, 98%, or at least 99%) identical to at least one of SEQ ID NOs: 1-30; sequences complementary to any such sequences, or subsequences thereof including at least 10 contiguous nucleotides of, e.g., SEQ ID NOs: 1-30 (or at least 12, 14, 16, 17 or more contiguous nucleotides of one of the designated sequences).

In one embodiment, plant growth is regulated by modulating expression or activity of at least one polypeptide contributing to a plant growth trait. The modulation of plant growth traits can be done in variety of plants, e.g., flowering plants, a member of the family of Brassicaceae, or Arabidopsis, Brassica, Zea, Oryza, Triticum, Hordeum, Lolium, Sorghum, Glycine, Medicago, Helianthus, Lactuca, Beta, Vitis, Solanum, Lycopersicon, Capsicum, Gossypium, Hevea, Linum, Prunus, Citrus, Populus, Pinus, Quercus, Aspergillus, Neurospora, Candida and Saccharomyces. In an embodiment, expression is modulated by expressing an exogenous nucleic acid including a polynucleotide sequence selected from SEQ ID NO: 1 to SEQ ID NO: 30. In other embodiments, expression of an endogenous nucleic acid, such as an endogenous nucleic acid encoding one of SEQ ID NO: 31 through SEQ ID NO: 60 is induced or suppressed, for example, by introducing, e.g., integrating, an exogenous nucleic acid including at least one promoter that regulates expression of the endogenous nucleic acid. In other embodiments, altered expression or activity of an expression product encoded by a nucleic acid, e.g., a polynucleotide sequence selected from SEQ ID NO: 1 to SEQ ID NO: 30 or conservative varients thereof, is detected, e.g., in a high throughput assay.

In some embodiments, expression or activity is modulated in response to an environmental factor, a chemical or biological agent, a pathogen, a bacteria, a virus, a fungus or an insect. An aspect of the invention includes methods which involve detecting altered expression or activity of an expression product, such as an RNA or polypeptide, encoded by a nucleic acid including a polynucleotide sequence selected from, e.g., SEQ ID NO: 1 to SEQ ID NO: 30. In some cases, altered expression or activity in response to the presence of a fertilizer or a herbicide is detected. In certain embodiments, a plurality of expression products are detected, e.g., in an array, a bead array or in a high-throughput assay.

In an embodiment, a data record related to the altered expression or activity is recorded in a database. For example, a data record can be a character string recorded in a data base made up of a plurality of character strings recorded in a computer or on a computer readable medium.

In another aspect, the invention provides methods for detecting genes for a plant growth trait. The methods of the invention for detecting genes for a plant growth trait involve providing a subject cell or tissue sample of nucleic acids and detecting at least one polynucleotide sequence or expression product corresponding to a polynucleotide sequence of the invention, e.g., such as a polynucleotide sequence selected from SEQ ID NO: 1-SEQ ID NO: 30, sequences that hybridize under stringent conditions to any one of SEQ ID NO: 1-SEQ ID NO: 30, sequences that are at least about 70% (or at least 75%, 80%, 85%, 90%, 95%, 97%, 98%, or at least 99%) identical to any one of SEQ ID NO: 1-SEQ ID NO: 30, sequences that encode a polypeptide encoded by any one of SEQ ID NO: 1-SEQ ID NO: 30, sequences complementary to any such sequences, or subsequences thereof including at least 10 contiguous nucleotides, e.g., of SEQ ID NOs: 1-30 (or at least 12, 14, 16, or 17 or more contiguous nucleotides of one of the designated sequences.

Detection of expression products is performed either qualitatively (presence or absence of one or more product of interest) or quantitatively (by monitoring the level of expression of one or more product of interest). In one embodiment, the expression product is an RNA expression product, such as differentially expressed RNA. The present invention optionally includes monitoring an expression level of a nucleic acid or polypeptide as noted herein for detection of a plant growth trait in a plant or in a population of plants.

Kits which incorporate one or more of the nucleic acids, polypeptides, antibodies, or arrays noted above are also a feature of the invention. Such kits can include any of the above noted components and further include, e.g., instructions for use of the components in any of the methods noted herein, packaging materials, containers for holding the components, and/or the like.

Digital systems which incorporate one or more representation (e.g., character string, data table, or the like) of one or more of the nucleic acids or polypeptides herein are also a feature of the invention.

### BRIEF DESCRIPTION OF THE DRAWINGS

FIG. 1 shows a chart of differential gene expression between a plant having long roots and a plant having short roots versus chromosome position. A QTL plot for association with root length is also mapped on the same genome.

FIG. 2 shows Arabidopsis QTL plots for three growth related traits (root length, aerial mass, and root mass). The LOD score for association of each marker interval in the genome with each phenotype is shown.

### DETAILED DISCUSSION

Control of plant growth is perhaps the most important goal in modern agriculture. The rate of plant growth, overall yield of usable plant mass, fertilizer response, and sensitivity to herbicides can all affect a farmer's productivity. First, the rate of plant growth can be critical, e.g., where growing seasons are short, where several crops are planted each year, or for long growing crops such as lumber. Second, maximum growth in the usable plant mass is desirable, e.g., in the roots of a potato plant, trunk of a pine tree, leaves of tobacco and grain of wheat. Third, growth modulation by application of fertilizers and herbicides must be efficient to reduce costs and to protect the environment. As a result, effective control of plant growth traits is central to productive agriculture.

Plant growth is a complex trait subject to complex interactions of genes and the environment. Multiple genes, e.g., metabolic, structural and tissue specific genes, interact to influence plant growth. Multiple environmental factors, e.g., availability of nutrients, light conditions, temperature, the presence of herbicides, availability of water, the presence of salts, etc., also play roles in plant growth. Finally, the multiple genetic and environmental factors interact to provide the ultimate plant growth trait. Thus, identification of genes associated with growth in plants can furnish tools to investigate interactions that can produce a desired plant growth trait.

The present invention provides genes associated with plant growth, which are useful tools in deciphering the complex interactions for improved plant growth. The provided genes can be employed directly, e.g., to produce recombinant plants with desired characteristics. The polynucleotides and polypeptides of the invention can be used as tools, e.g., as elements of marker sets, sequence databases, probes, enzymes, and processes, to investigate interactions resulting in desired growth traits.

Definitions

Unless defined otherwise, all scientific and technical terms are understood to have the same meaning as commonly used in the art to which they pertain. For the purpose of the present invention, the following terms are defined below.

The term plant growth trait refers to quantifiable plant growth parameters such as, e.g., root length, aerial mass, root mass, total plant mass, stem growth rate, etc.

The term “nucleic acid” is generally used in its art-recognized meaning to refer to a ribose nucleic acid (RNA) or deoxyribose nucleic acid (DNA) polymer, or analog thereof, e.g., a nucleotide polymer comprising modifications of the nucleotides, a peptide nucleic acid, or the like. In certain applications, the nucleic acid can be a polymer that includes both RNA and DNA subunits. A nucleic acid can be, e.g., a chromosome or chromosomal segment, a vector (e.g., an expression vector), a naked DNA or RNA polymer, the product of a polymerase chain reaction (PCR), an oligonucleotide, a probe, etc.

The term “polynucleotide sequence” refers to a contiguous sequence of nucleotides in a single nucleic acid or to a representation, e.g., a character string, thereof. “Polymorphic polynucleotides” are polynucleotide sequences corresponding to a single locus, i.e., alleles at a locus, characterized by at least one variant (or alternative) nucleotide subunit. Thus, a polymorphic polynucleotide is a polynucleotide that differs, e.g., from another allele at the same locus, or between an otherwise homologous or similar polynucleotide, at one or more nucleotide positions.

A “phenotype” is the display of a trait in an individual organism resulting from the interaction of gene expression and the environment.

An “expression vector” is a vector, e.g., a plasmid, capable of producing transcripts and, potentially, polypeptides encoded by a polynucleotide sequence. Typically, an expression vector is capable of producing transcripts in an exogenous cell, e.g., a bacterial cell, or a plant cell, in vivo or invitro, e.g., a cultured plant protoplast. Expression of a product can be either constitutive or inducible depending, e.g., on the promoter selected. In the context of an expression vector, a promoter is said to be “operably linked” to a polynucleotide sequence if it is capable of regulating expression of the associated polynucleotide sequence. The term also applies to alternative exogenous gene constructs, such as expressed or integrated transgenes. Similarly, the term operably linked applies equally to alternative or additional transcriptional regulatory sequences such as enhancers, associated with a polynucleotide sequence.

An “expression product” is a transcribed sense or antisense RNA, or a translated polypeptide corresponding to a polynucleotide sequence. Depending on context, the term also can be used to refer to an amplification product (amplicon) or cDNA corresponding to the RNA expression product transcribed from the polynucleotide sequence.

A polynucleotide sequence is said to “encode” a sense or antisense RNA molecule, or a polypeptide, if the polynucleotide sequence can be transcribed (in spliced or unspliced form) or translated into the RNA or polypeptide, or a fragment of thereof.

A probe and a gene (or expression product) are said to “correspond” when they share substantial structural identity, or complimentarity, depending on context. For example, a probe or an expression product, e.g., a messenger RNA, corresponds to a gene when it is derived from a genetic element with substantial sequence identity.

Polynucleotides of the Invention

The present invention is based on the identification of nucleic acid sequences and full length genes associated with control of growth traits in plants. The gene sequences of the invention can influence plant growth by their presence in the genome of a plant species or by the abundance of their expression products in such a plant.

The sequences of the invention can be implicated in control of plant growth traits in their differential expression between plants with high growth and low growth characteristics. The specified sequences can be implicated in the control of growth traits in plants by their differential regulation in response to environmental factors known to induce or suppress display of the growth traits. Unlike the vast majority of polynucleotide sequences present in the plant genome, e.g., randomly selected unique or repetitive polynucleotide sequences, this defined and limited group of polynucleotides, possess an extraordinary high probability of association with loci involved in the growth traits in plants.

Given the sequences of the invention, as disclosed herein, those skilled in the art can readily synthesize the sequences or screen them from nature. Screening from nature can be, e.g., by massively parallel signature sequencing (MPSS). Massively parallel signature sequencing is a wide ranging and sensitive quantitative cDNA analysis tool for preparation of expression profiles, Brenner et al. “In vitro cloning of complex mixtures of DNA on microbeads: Physical separation of differentially expressed cDNAs”, (2000) PNAS 97, 1665-1670. In MPSS, cDNA is prepared from poly(A) RNA (mRNA) using a biotin-labeled oligo-dT primer. The oligo-dT is designed to prime each mRNA molecule exactly at the poly(A) junction. The cDNA fragments are then digested with DpnII (recognition sequence GATC), and the 3′-most DpnII-poly(A) fragments are purified utilizing the biotin label at the end of each molecule. The fragments are subsequently bound to 5 micron diameter microbeads using a complex set of 32 base tag/antitags. This process yields a library of beads where one mRNA molecule is represented by one microbead, and each microbead contains approximately 100,000 identical cDNA fragments from that mRNA. All molecules are covalently attached to the microbeads at their poly(A) ends; therefore, the DPNII end is available for sequencing reactions. Expression differences between organisms, e.g., of different phenotypes can be identified using MPSS as a tool.

Accordingly, in one aspect, the polynucleotide sequences of the invention are useful for identifying corresponding cDNAs associated with growth in plants and/or chromosomal segments associated with growth. More generally, the polynucleotide sequences of the invention and corresponding polypeptides are useful, individually and/or collectively, as probes (e.g., probes labeled with a detectable moiety) and markers. In addition, the polynucleotide sequences of the invention are useful for the production of plant and cell culture models useful for the monitoring of agents and evaluation of protocols aimed at controlling growth in plants. Nucleic acid sequences of the invention, e.g., SEQ ID NO: 1 through SEQ ID NO: 30, can also be used in vector systems to control plant growth, e.g., by transformation of plant cells to modulate expression of growth correlated genes.

Polynucleotide sequences of the invention include, e.g., the polynucleotide sequences represented by SEQ ID NO: 1 through SEQ ID NO: 30 and SEQ ID NO: 61 through SEQ ID NO: 403. In addition to the sequences expressly provided in the accompanying sequence listing, the invention includes polynucleotide sequences, that are highly related structurally and/or functionally. For example, polynucleotides encoding polypeptide sequences represented by SEQ ID NO: 31 through SEQ ID NO: 60, or subsequences thereof are one embodiment of the invention. In addition, polynucleotide sequences of the invention include polynucleotide sequences that hybridize under stringent conditions to a polynucleotide sequence comprising any of SEQ ID NO: 1-SEQ ID NO: 30.

In addition to the polynucleotide sequences of the invention, e.g., enumerated in SEQ ID NO: 1 to SEQ ID NO: 30, or SEQ ID NO: 61-SEQ ID NO: 403, polynucleotide sequences that are substantially identical to a polynucleotide of the invention can be used in the compositions and methods of the invention. Substantially identical or substantially similar polynucleotide (or polypeptide) sequences are defined as polynucleotide (or polypeptide) sequences that are identical, on a nucleotide by nucleotide bases, with at least a subsequence of a reference polynucleotide (or polypeptide), e.g., selected from SEQ ID NO: 1-30 (or 61-403). Such polynucleotides can include, e.g., insertions, deletions, and substitutions relative to any of SEQ ID NO: 1-30. For example, such polynucleotides are typically at least about 70% identical to a reference polynucleotide (or polypeptide) selected from among SEQ ID NO: 1 through SEQ ID NO: 30 (or 61-403). That is, at least 7 out of 10 nucleotides (or amino acids) within a window of comparison are identical to the reference sequence selected SEQ ID NO: 1-30. Frequently, such sequences are at least about 80%, usually at least about 90%, and often at least about 95%, or even at least about 98%, or about 99%, identical to the reference sequence, e.g., at least one of SEQ ID NO: 1 to SEQ ID NO: 30 or SEQ ID NO: 61 to SEQ ID NO: 403.

Subsequences of the polynucleotides of the invention described above, e.g., SEQ ID NOs: 1-30, including at least 10 contiguous nucleotides or complementary subsequences thereof are also a feature of the invention. More commonly a subsequence includes at least 12 contiguous nucleotides, e.g.;, of one or more of SEQ ID NO: 1 through SEQ ID NO: 30 or SEQ ID NO: 61 through SEQ ID NO: 403. Typically, the subsequence includes at least 14, frequently at least 16, and usually at least 17 or more contiguous nucleotides of one of the specified polynucleotide sequences. Such subsequences can be, e.g., oligonucleotides, such as synthetic oligonucleotides, or full-length genes or cDNAs.

In addition, polynucleotide sequences complementary to any of the above described sequences are included among the polynucleotides of the invention. Where the polynucleotide sequences are translated to form a polypeptide or subsequence of a polypeptide, the nucleotide changes can result in either conservative or non-conservative amino acid substitutions. Conservative amino acid substitutions refer to the interchangeability of residues having functionally similar side chains. Conservative substitution tables providing functionally similar amino acids are well known in the art. Table 1 sets forth six groups which contain amino acids that are “conservative substitutions” for one another. Other conservative substitution charts are available in the art, and can be used in a similar manner.

TABLE 1
Conservative Substitution Group

| | | | | |
|---|---|---|---|---|
| 1 | Alanine (A) | Serine (S) | Threonine (T) | |
| 2 | Aspartic acid (D) | Glutamic acid (E) | | |
| 3 | Asparagine (N) | Glutamine (Q) | | |
| 4 | Arginine (R) | Lysine (K) | | |
| 5 | Isoleucine (I) | Leucine (L) | Methionine (M) | Valine (V) |
| 6 | Phenylalanine (F) | Tyrosine (Y) | Tryptophan (W) | |

One of skill in the art will appreciate that many conservative substitutions of the nucleic acid constructs which are disclosed yield a functionally identical construct. For example, as discussed above, owing to the degeneracy of the genetic code, “silent substitutions” (i.e., substitutions in a nucleic acid sequence which do not result in an alteration in an encoded polypeptide) are an implied feature of every nucleic acid sequence which encodes an amino acid. Similarly, “conservative amino acid substitutions,” in one or a few amino acids in an amino acid sequence (e.g., about 1%, 2%, 3%, 4%, 5%, 6%, 7%, 8%, 9%, 10% or more) are substituted with different amino acids with highly similar properties, are also readily identified as being highly similar to a disclosed construct. Such conservative variations of each disclosed sequence are a feature of the present invention.

Methods for obtaining conservative variants, as well as more divergent versions of the nucleic acids and polypeptides of the invention are widely known in the art. In addition to naturally occurring homologues which can be obtained, e.g., by screening genomic or expression libraries according to any of a variety of well-established protocols, see, e.g., Ausubel et al. Current Protocols in Molecular Biology (supplemented through 2001) John Wiley & Sons, New York (“Ausubel”); Sambrook et al. Molecular Cloning—A Laboratory Manual (2nd Ed.), Vol. 1-3, Cold Spring Harbor Laboratory, Cold Spring Harbor, N.Y., 1989 (“Sambrook”), and Berger and Kimmel Guide to Molecular Cloning Techniques, Methods in Enzymology volume 152 Academic Press, Inc., San Diego, Calif. (“Berger”), additional variants can be produced by a variety of mutagenesis procedures. Many such procedures are known in the art, including site directed mutagenesis, oligonucleotide-directed mutagenesis, and many others. For example, site directed mutagenesis is described, e.g., in Smith (1985) “In vitro mutagenesis” Ann. Rev. Genet. 19:423-462, and references therein, Botstein & Shortle (1985) “Strategies and applications of in vitro mutagenesis” Science 229:1193-1201; and Carter (1986) “Site-directed mutagenesis” Biochem. J. 237:1-7. Oligonucleotide-directed mutagenesis is described, e.g., in Zoller & Smith (1982) “Oligonucleotide-directed mutagenesis using M13-derived vectors: an efficient and general procedure for the production of point mutations in any DNA fragment” Nucleic Acids Res. 10:6487-6500). Mutagenesis using modified bases is described e.g., in Kunkel (1985) “Rapid and efficient site-specific mutagenesis without phenotypic selection” Proc. Natl. Acad. Sci. USA 82:488-492, and Taylor et al. (1985) “The rapid generation of oligonucleotide-directed mutations at high frequency using phosphorothioate-modified DNA” Nucl. Acids Res. 13: 8765-8787. Mutagenesis using gapped duplex DNA is described, e.g., in Kramer et al. (1984) “The gapped duplex DNA approach to oligonucleotide-directed mutation construction” Nucl. Acids Res. 12: 9441-9460). Point mismatch repair is described, e.g., by Kramer et al. (1984) “Point Mismatch Repair” Cell 38:879-887). Double-strand break repair is described, e.g., in Mandecki (1986) “Oligonucleotide-directed double-strand break repair in plasmids of Escherichia coli: a method for site-specific mutagenesis” Proc. Natl. Acad. Sci. USA, 83:7177-7181, and in Arnold (1993) “Protein engineering for unusual environments” Current Opinion in Biotechnology 4:450-455). Mutagenesis using repair-deficient host strains is described, e.g., in Carter et al. (1985) “Improved oligonucleotide site-directed mutagenesis using M13 vectors” Nucl. Acids Res. 13: 4431-4443. Mutagenesis by total gene synthesis is described e.g., by Nambiar et al. (1984) “Total synthesis and cloning of a gene coding for the ribonuclease S protein” Science 223: 1299-1301. DNA shuffling is described, e.g., by Stemmer (1994) “Rapid evolution of a protein in vitro by DNA shuffling” Nature 370:389-391, and Stemmer (1994) “DNA shuffling by random fragmentation and reassembly: In vitro recombination for molecular evolution.” Proc. Natl. Acad. Sci. USA 91:10747-10751.

Many of the above methods are further described in Methods in Enzymology Volume 154, which also describes useful controls for trouble-shooting problems with various mutagenesis methods. Kits for mutagenesis, library construction and other diversity generation methods are also commercially available. For example, kits are available from, e.g., Amersham International plc (e.g., using the Eckstein method above), Anglian Biotechnology Ltd (e.g., using the Carter/Winter method above), Bio/Can Scientific, Bio-Rad (e.g., using the Kunkel method described above), Boehringer Mannheim Corp., Clonetech Laboratories, DNA Technologies, Epicentre Technologies (e.g., the 5 prime 3 prime kit); Genpak Inc, Lemargo Inc, Life Technologies (Gibco BRL), New England Biolabs, Pharmacia Biotech, Promega Corp., Quantum Biotechnologies, Stratagene (e.g., QuickChange™ site-directed mutagenesis kit; and Chameleon™ double-stranded, site-directed mutagenesis kit).

Determining Sequence Relationships

The nucleic acid and amino acid sequences of the invention include, e.g., those provided in SEQ ID NO: 1 to SEQ ID NO: 403 as well as similar sequences. Similar sequences are objectively determined by any number of methods, e.g., percent identity, hybridization, immunologically, and the like. A variety of methods for determining relationships between two or more sequences (e.g., identity, similarity and/or homology) are available, and well known in the art. The methods include manual alignment, computer assisted sequence alignment and combinations thereof. A number of algorithms (which are generally computer implemented) for performing sequence alignment are widely available, or can be produced by one of skill. These methods include, e.g., the local homology algorithm of Smith and Waterman (1981) Adv. Appl. Math. 2:482; the homology alignment algorithm of Needleman and Wunsch (1970) J. Mol. Biol. 48:443; the search for similarity method of Pearson and Lipman (1988) Proc. Natl. Acad. Sci. (USA) 85:2444; and/or by computerized implementations of these algorithms (e.g., GAP, BESTFIT, FASTA, and TFASTA in the Wisconsin Genetics Software Package Release 7.0, Genetics Computer Group, 575 Science Dr., Madison, Wis.).

For example, software for performing sequence identity (and sequence similarity) analysis using the BLAST algorithm is described in Altschul et al. (1990) J. Mol. Biol. 215:403-410. This software is publicly available, e.g., through the National Center for Biotechnology Information on the world wide web at ncbi.nlm.nih.gov. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP (BLAST Protein) program uses as defaults a wordlength (W) of 3, an expectation (E) of 10, and the BLOSUM62 scoring matrix (see, Henikoff & Henikoff (1989) Proc. Natl. Acad. Sci. USA 89:10915).

Additionally, the BLAST algorithm performs a statistical analysis of the similarity between two sequences (see, e.g., Karlin & Altschul (1993) Proc. Nat'l. Acad. Sci. USA 90:5873-5787). One measure of similarity provided by the BLAST algorithm is the smallest sum probability (p(N)), which provides an indication of the probability by which a match between two nucleotide or amino acid sequences would occur by chance. For example, a nucleic acid is considered similar to a reference sequence (and, therefore, in this context, homologous) if the smallest sum probability in a comparison of the test nucleic acid to the reference nucleic acid is less than about 0.1, or less than about 0.01, and or even less than about 0.001.

Another example of a useful sequence alignment algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS5:151-153. The program can align, e.g., up to 300 sequences of a maximum length of 5,000 letters. The multiple alignment procedure begins with the pairwise alignment of the two most similar sequences, producing a cluster of two aligned sequences. This cluster can then be aligned to the next most related sequence or cluster of aligned sequences. Two clusters of sequences can be aligned by a simple extension of the pairwise alignment of two individual sequences. The final alignment is achieved by a series of progressive, pairwise alignments. The program can also be used to plot a dendogram or tree representation of clustering relationships. The program is run by designating specific sequences and their amino acid or nucleotide coordinates for regions of sequence comparison.

An additional example of an algorithm that is suitable for multiple DNA, or amino acid, sequence alignments is the CLUSTALW program (Thompson, J. D. et al. (1994) Nucl. Acids. Res. 22: 4673-4680). CLUSTALW performs multiple pairwise comparisons between groups of sequences and assembles them into a multiple alignment based on homology. Gap open and Gap extension penalties can be, e.g., 10 and 0.05 respectively. For amino acid alignments, the BLOSUM algorithm can be used as a protein weight matrix. See, e.g., Henikoff and Henikoff (1992) Proc. Natl. Acad. Sci. USA 89: 10915-10919.

Nucleic Acid Hybridization

Similarity between nucleic acids of the invention can also be evaluated by “hybridization” between single stranded (or single stranded regions of) nucleic acids with complementary or partially complementary polynucleotide sequences.

Hybridization is a measure of the physical association between nucleic acids, typically, in solution, or with one of the nucleic acid strands immobilized on a solid support, e.g., a membrane, a bead, a chip, a filter, etc. Nucleic acid hybridization occurs based on a variety of well characterized physico-chemical forces, such as hydrogen bonding, solvent exclusion, base stacking, and the like. Numerous protocols for nucleic acid hybridization are well known in the art. An extensive guide to the hybridization of nucleic acids is found in Tijssen (1993) Laboratory Techniques in Biochemistry and Molecular Biology—Hybridization with Nucleic Acid Probes, part I, chapter 2, “Overview of principles of hybridization and the strategy of nucleic acid probe assays,” (Elsevier, New York), as well as in Ausubel et al. Current Protocols in Molecular Biology (supplemented through 2001) John Wiley & Sons, New York (“Ausubel”); Sambrook et al. Molecular Cloning—A Laboratory Manual (2nd Ed.), Vol. 1-3, Cold Spring Harbor Laboratory, Cold Spring Harbor, N.Y., 1989 (“Sambrook”), and Berger and Kimmel Guide to Molecular Cloning Techniques, Methods in Enzymology volume 152 Academic Press, Inc., San Diego, Calif. (“Berger”). Hames and Higgins (1995) Gene Probes 1, IRL Press at Oxford University Press, Oxford, England (Hames and Higgins 1) and Hames and Higgins (1995) Gene Probes 2, IRL Press at Oxford University Press, Oxford, England (Hames and Higgins 2) provide details on the synthesis, labeling, detection and quantification of DNA and RNA, including oligonucleotides.

Conditions suitable for obtaining hybridization, including differential hybridization, are selected according to the theoretical melting temperature (Tₘ) between complementary and partially complementary nucleic acids. Under a given set of conditions, e.g., solvent composition, ionic strength, etc., the Tₘ is the temperature at which the duplex between the hybridizing nucleic acid strands is 50% denatured. That is, the Tₘ corresponds to the temperature corresponding to the midpoint in transition from helix to random coil; it depends on the length of the nucleotides, nucleotide composition, and ionic strength, for long stretches of nucleotides.

After hybridization, unhybridized nucleic acids can be removed by a series of washes, the stringency of which can be adjusted depending upon the desired results. Low stringency washing conditions (e.g., using higher salt and lower temperature) increase sensitivity, but can product nonspecific hybridization signals and high background signals. Higher stringency conditions (e.g., using lower salt and higher temperature that is closer to the Tₘ) lower the background signal, typically with primarily the specific signal remaining. See, also, Rapley, R. and Walker, J. M. eds., Molecular Biomethods Handbook (Humana Press, Inc. 1998).

“Stringent hybridization wash conditions” or “stringent conditions” in the context of nucleic acid hybridization experiments, such as Southern and northern hybridizations, are sequence dependent, and are different under different environmental parameters. An extensive guide to the hybridization of nucleic acids is found in Tijssen (1993), supra, and in Hames and Higgins 1 and Hames and Higgins 2, supra.

An example of stringent hybridization conditions for hybridization of complementary nucleic acids which have more than 100 complementary residues on a filter in a Southern or northern blot is 2× SSC, 50% formamide at 42° C., with the hybridization being carried out overnight (e.g., for approximately 20 hours). An example of stringent wash conditions is a 0.2× SSC wash at 65° C. for 15 minutes (see Sambrook, supra for a description of SSC buffer). Often, the wash determining the stringency is preceded by a low stringency wash to remove signal due to residual unhybridized probe. An example low stringency wash is 2× SSC at room temperature (e.g., 20° C. for 15 minutes).

In general, a signal to noise ratio of at least 2.5×-5× (and typically higher) than that observed for an unrelated probe in the particular hybridization assay indicates detection of a specific hybridization. Detection of at least stringent hybridization between two sequences in the context of the present invention indicates relatively strong structural similarity to, e.g., the nucleic acids of the present invention provided in the sequence listings herein.

For purposes of the present invention, generally, “highly stringent” hybridization and wash conditions are selected to be about 5° C. or less lower than the thermal melting point (Tₘ) for the specific sequence at a defined ionic strength and pH (as noted below, highly stringent conditions can also be referred to in comparative terms). Target sequences that are closely related or identical to the nucleotide sequence of interest (e.g., “probe”) can be identified under stringent or highly stringent conditions. Lower stringency conditions are appropriate for sequences that are less complementary.

For example, in determining stringent or highly stringent hybridization (or even more stringent hybridization) and wash conditions, the hybridization and wash conditions are gradually increased (e.g., by increasing temperature, decreasing salt concentration, increasing detergent concentration, and/or increasing the concentration of organic solvents, such as formamide, in the hybridization or wash), until a selected set of criteria are met. For example, the hybridization and wash conditions are gradually increased until a probe comprising one or more polynucleotide sequences of the invention, e.g., selected from SEQ ID NO: 1 to SEQ ID NO: 30, SEQ ID NO: 61 through SEQ ID NO: 403, and/or complementary polynucleotide sequences thereof, binds to a perfectly matched complementary target (again, a nucleic acid comprising one or more nucleic acid sequences or subsequences selected from SEQ ID NO: 1 to SEQ ID NO: 30, SEQ ID NO: 61 through SEQ ID NO: 403, and complementary polynucleotide sequences thereof), with a signal to noise ratio that is at least 2.5×, and optionally 5×, or 10×, or 100× or more, as high as that observed for hybridization of the probe to an unmatched target, as desired.

For example, using subsequences derived from the nucleic acids encoding the polypeptides of the invention, novel target nucleic acids can be obtained; such target nucleic acids are also a feature of the invention. For example, such target nucleic acids include sequences that hybridize under stringent conditions to an oligonucleotide probe that encodes a unique subsequence in any of the polypeptides of the invention, e.g., SEQ ID NOs: 31-60.

For example, hybridization conditions are chosen under which a target oligonucleotide that is perfectly complementary to the oligonucleotide probe hybridizes to the probe with at least about a 5-10× higher signal to noise ratio than for hybridization of the target oligonucleotide to a negative control non-complimentary nucleic acid.

Higher ratios of signal to noise can be achieved by increasing the stringency of the hybridization conditions such that ratios of about 15×, 20×, 30×, 50× or more are obtained. The particular signal will depend on the label used in the relevant assay, e.g., a fluorescent label, a calorimetric label, a radio active label, or the like.

Probes

Nucleic acids including one or more polynucleotide sequence of the invention are favorably used as probes for the detection of complimentary, corresponding, or related nucleic acids in a variety of contexts, such as the nucleic hybridization experiments discussed above. The probes can be either DNA or RNA molecules, such as restriction fragments of genomic or cloned DNA, cDNAs, amplification products, transcripts, and oligonucleotides, and can vary in length from oligonucleotides as short as about 10 nucleotides in length to chromosomal fragments or cDNAs in excess of one or more kilobases. For example, in some embodiments, a probe of the invention includes a polynucleotide sequence or subsequence selected from among SEQ ID NO: 1 to SEQ ID NO: 30, SEQ ID NO: 61 through SEQ ID NO: 403, or sequences complementary thereto. Alternatively, polynucleotide sequences that are variants of one of the above designated sequences can be used as probes. Most typically, such variants include one or a few nucleotide variations. For example, pairs (or sets) of oligonucleotides can be selected, in which the two (or more) polynucleotide sequences are conservative variations of each other, wherein one polynucleotide sequence corresponds identically to a first allele or allelic variant and the other(s) correspond identically to additional alleles or allelic variants. Such pairs of oligonucleotide probes are particularly useful, e.g., for allele specific hybridization experiments to detect polymorphic nucleotides. In other applications, probes are selected that are more divergent, that is, probes that are at least about 70% (or 80%, 90%, 95%, 98%, or 99%) identical are selected.

The probes of the invention, as exemplified by sequences derived from SEQ ID NO: 1 through SEQ ID NO: 30 and SEQ ID NO: 61 through SEQ ID NO: 403, can also be used to identify additional useful polynucleotide sequences according to procedures routine in the art. In one set of embodiments, one or more probes, as described above, are utilized to screen libraries of expression products or chromosomal segments (e.g. expression libraries or genomic libraries) to identify clones that include sequences identical to, or with significant sequence similarity to, one or more of SEQ ID NO: 1-30, i.e., allelic variants, homologues or orthologues. In turn, each of these identified sequences can be used to make probes, including pairs or sets of variant probes as described above. It will be understood that in addition to such physical methods as library screening, computer assisted bioinformatic approaches, e.g., BLAST and other sequence homology search algorithms, and the like, can also be used for identifying related polynucleotide sequences. Polynucleotide sequences identified in this manner are also a feature of the invention.

For example, oligonucleotide probes, most typically produced by well known synthetic methods, such as the solid phase phosphoramidite triester method described by Beaucage and Caruthers (1981) Tetrahedron Letts. 22(20):1859-1862, e.g., using an automated synthesizer, as described in Needham-VanDevanter et al. (1984) Nucleic Acids Res., 12:6159-6168. Oligonucleotides can also be custom made and ordered from a variety of commercial sources known to persons of skill. Purification of oligonucleotides, where necessary, is typically performed by either native acrylamide gel electrophoresis or by anion-exchange UPLC as described in Pearson and Regnier (1983) J. Chrom. 255:137-149. The sequence of the synthetic oligonucleotides can be verified using the chemical degradation method of Maxam and Gilbert (1980) in Grossman and Moldave (eds.) Academic Press, New York, Methods in Enzymology 65:499-560. Custom oligos can also easily be ordered from a variety of commercial sources known to persons of skill.

In addition, essentially any nucleic acid can be custom ordered from any of a variety of commercial sources, such as The Midland Certified Reagent Company (mcrc@oligos.com), The Great American Gene Company (http:Hlwww.genco.com), ExpressGen Inc. (www.expressgen.com), Operon Technologies Inc. (Alameda, Calif.) and many others. Similarly, peptides and antibodies can be custom ordered from any of a variety of sources, such as PeptidoGenic (pkim@ccnet.com), HTI Bio-products, inc. (http:/Iwww.htibio.com), BMA Biomedicals Ltd (U.K.), Bio.Synthesis, Inc., and many others.

As noted, in one embodiment, oligonucleotide probes of the invention include subsequences of SEQ ID NO: 1 through SEQ ID NO: 30, SEQ ID NO: 61 through SEQ ID NO: 403, and/or complementary sequences thereof, including e.g., at least 10 contiguous nucleotides in length. Commonly, the oligonucleotide probes are at least 12 contiguous nucleotides in length; usually, the oligonucleotides are at least 14 contiguous nucleotides in length; frequently, the oligonucleotides are at least 16 contiguous nucleotides in length, and in many cases the oligonucleotides are at least 17 or more contiguous nucleotides of at least one sequence selected from SEQ ID NO: 1 to SEQ ID NO: 30 or SEQ ID NO: 61 through SEQ ID NO: 403. In some cases, the oligonucleotide probes consist of a polynucleotide sequence selected from SEQ ID NO: 1 through SEQ ID NO: 30 or from SEQ ID NO: 61 through SEQ ID NO: 403.

In other circumstances, e.g., relating to functional attributes of cells or organisms expressing the polynucleotides and polypeptides of the invention, probes that are polypeptides, peptides, or antibodies are favorably utilized. For example, polypeptides, polypeptide fragments, and peptides corresponding to, or derived from SEQ ID NO: 31 to SEQ ID NO: 60, are favorably used to identify and isolate antibodies or other binding proteins, e.g., from phage display libraries, combinatorial libraries, polyclonal sera, and the like.

Antibodies specific for any one of SEQ ID NO: 31 to SEQ ID NO: 60 are likewise valuable as probes for evaluating expression products, e.g., from cells or tissues. In addition, antibodies are particularly suitable for evaluating expression of proteins corresponding to SEQ ID NOs: 31-60, in situ, in a cell, tissue or whole plant, e.g., a plant providing an experimental model for manipulation of growth traits. Antibodies can be directly labeled with a detectable reagent as described below, or detected indirectly by labeling of a secondary antibody specific for the heavy chain constant region (i.e., isotype) of the specific antibody. Additional details regarding production of specific antibodies are provided below in the section entitled “Antibodies.”

Labeling and Detecting Probes

Numerous methods are available for labeling and detection of the nucleic acid and polypeptide (or peptide or antibody) probes of the invention, these include: 1) fluorescence (using, e.g., fluorescein, Cy-5, rhodamine or other fluorescent tags); 2) isotopic methods, e.g., using end-labeling, nick translation, random priming, or PCR to incorporate radioactive isotopes into the probe polynucleotide/oligonucleotide; 3) chemifluorescence using alkaline phosphatase and the substrate AttoPhos (Amersham) or other substrates that produce fluorescent products; 4) chemiluminescence (using either horseradish peroxidase and/or alkaline phosphatase with substrates that produce photons as breakdown products, kits providing reagents and protocols are available from such commercial sources as Amersham, Boehringer-Mannheim, and Life Technologies/Gibco BRL); and, 5) colorimetric methods (again using both horseradish peroxidase and alkaline phosphatase with substrates that produce a colored precipitate, kits are available from Life Technologies/Gibco BRL, and Boehringer-Mannheim). Other methods for labeling and detection will be readily apparent to one skilled in the art.

More generally, a probe can be labeled with any composition detectable by spectroscopic, photochemical, biochemical, immunochemical, electrical, optical, chemical, or other available means. Useful labels in the present invention include spectral labels such as fluorescent dyes (e.g., fluorescein isothiocyanate, Texas red, rhodamine, and the like), radiolabels (e.g., ³H, ¹²⁵I, ³⁵S, ¹⁴C, ³²P, ³³P, etc.), enzymes (e.g., horse-radish peroxidase, alkaline phosphatase, etc.), spectral colorimetric labels such as colloidal gold, or colored glass or plastic (e.g. polystyrene, polypropylene, latex, etc.) beads. The label may be coupled directly or indirectly to a component of the detection assay (e.g., a probe, such as an oligonucleotide, isolated DNA, amplicon, restriction fragment, or the like) according to methods well known in the art. As indicated above, a wide variety of labels may be used, with the choice of label depending on sensitivity required, ease of conjugation with the compound, stability requirements, available instrumentation, and disposal provisions. In general, a detector which monitors a probe-target nucleic acid hybridization is adapted to the particular label which is used. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising a nucleic acid array with particular set of probes bound to the array is digitized for subsequent computer analysis.

Because incorporation of radiolabeled nucleotides into nucleic acids is straightforward, this detection represents one favorable labeling strategy. Exemplar technologies for incorporating radiolabels include end-labeling with a kinase or phoshpatase enzyme, nick translation, incorporation of radio-active nucleotides with a polymerase and many other well known strategies.

Fluorescent labels are desirable, having the advantage of requiring fewer precautions in handling, and being amenable to high-throughput visualization techniques. Preferred labels are typically characterized by one or more of the following: high sensitivity, high stability, low background, low environmental sensitivity and high specificity in labeling. Fluorescent moieties, which are incorporated into the labels of the invention, are generally are known, including Texas red, fluorescein isothiocyanate, rhodamine, etc. Many fluorescent tags are commercially available from SIGMA chemical company (Saint Louis, Mo.), Molecular Probes (Eugene, Oreg.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), and Applied Biosystems (Foster City, Calif.) as well as other commercial sources known to one of skill. Similarly, moieties such as digoxygenin and biotin, which are not themselves fluorescent but are readily used in conjunction with secondary reagents, i.e., anti-digoxygenin antibodies, avidin (or streptavidin), that can be labeled, are suitable as labeling reagents in the context of the probes of the invention.

The label is coupled directly or indirectly to a molecule to be detected (a product, substrate, enzyme, or the like) according to methods well known in the art. As indicated above, a wide variety of labels are used, with the choice of label depending on the sensitivity required, ease of conjugation of the compound, stability requirements, available instrumentation, and disposal provisions. Non-radioactive labels are often attached by indirect means. Generally, a ligand molecule (e.g., biotin) is covalently bound to a nucleic acid such as a probe, primer, amplicon, or the like. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with labeled, anti-ligands. Alternatively, any haptenic or antigenic compound can be used in combination with an antibody. Labels can also be conjugated directly to signal generating compounds, e.g., by conjugation with an enzyme or fluorophore or chromophore. Enzymes of interest a labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. Means of detecting labels are well known to those of skill in the art. Thus, for example, where the label is a radioactive label, means for detection include a scintillation counter or photographic film as in autoradiography. Where the label is optically detectable, typical detectors include microscopes, cameras, phototubes and photodiodes and many other detection systems which are widely available.

It will be appreciated that probe design is influenced by the intended application. For example, where several allele-specific probe-target interactions are to be detected in a single assay, e.g., on a single DNA chip, it is desirable to have similar melting temperatures for all of the probes. Accordingly, the length of the probes are adjusted so that the melting temperatures for all of the probes on the array are closely similar (it will be appreciated that different lengths for different probes may be needed to achieve a particular Tm where different probes have different GC contents). Although melting temperature is a primary consideration in probe design, other factors are optionally used to further adjust probe construction, such as selecting against primer self-complementarity and the like.

Marker Sets

Sets of probes, including multiple nucleic acids with polynucleotide sequences or sequences selected from among the polynucleotides of the invention, e.g., SEQ ID NO: 1 through SEQ ID NO: 30, SEQ ID NO: 61 through SEQ ID NO: 403, or subsequences thereof, or conservative variants thereof, or sequences complimentary to any of the foregoing are also a feature of the invention. Such sets of probes are useful as marker sets, e.g., for predicting plant growth traits before they become apparent, identifying plant or cell phenotype, and/or the like.

Marker sets of the invention favorably include any of the probe sequences described above, such as polynucleotide sequences that hybridize under stringent conditions to any one of SEQ ID NO: 1-SEQ ID NO: 30, any one of SEQ ID NO: 61 through SEQ ID NO: 403, sequences that are at least 70% identical to any one of SEQ ID NO: 1-SEQ ID NO: 30, sequences that encode a polypeptide or peptide comprising a subsequence encoded by any one of SEQ ID NO: 31-SEQ ID NO: 60, sequences complementary to any such sequences, or subsequences thereof.

In one embodiment, the marker set of the invention is a plurality of oligonucleotides, e.g., synthetic oligonucleotides produced by the phosporamidite triester synthesis method on an automated synthesizer, as described above. For example, at least two oligonucleotides including a polynucleotide sequence of at least 10 contiguous nucleotides of sequences selected from a polynucleotide of the invention, e.g., SEQ ID NO: 1 to SEQ ID NO: 30 or SEQ ID NO: 61 through SEQ ID NO: 403, can be used as a set to predict plant growth traits before they become apparent. Frequently, the oligonucleotides selected will be longer than 10 contiguous nucleotides in length, for example, oligonucleotides of at least 12, or 14, or 16 or 17, or more contiguous nucleotides are favorably employed in the marker sets of the invention.

While as few as one or two probes can constitute a marker set, it is frequently desirable to employ marker sets with more than two members. Typically, a marker set of the invention has at least 3, often at least about 5 or more members selected from among any of the polynucleotides of the invention. In one favorable embodiment, the marker set includes oligonucleotides corresponding in sequence to at least part of each of SEQ ID NO: 1 through SEQ ID NO: 30 or SEQ ID NO: 61 through SEQ ID NO: 403. In another embodiment, the marker sets are made up of expression products such as cDNAs, or amplification products corresponding to cDNA or RNA expression products.

In some applications, the marker set includes labeled nucleic acid probes as described in the preceding section. In other applications, e.g., certain array applications, a labeled nucleic acid sample is hybridized to a set of unlabeled marker nucleic acids.

The marker sets of the invention are frequently employed in the context of a polynucleotide sequence array. Any of the polynucleotide sequences of the invention, as described above, can be logically or physically arrayed to produce a useful array. For example, nucleic acids, e.g., oligonucleotides, cDNAs, amplicons, and/or chromosomal segments, can be physically arrayed in a solid phase or liquid phase array. Common solid phase arrays include a variety of solid substrates suitable for attaching nucleic acids in an ordered manner, such as membranes, filters, chips, beads, pins, slides, plates, etc. Common liquid phase arrays include, e.g., arrays of wells (e.g., as in microtiter trays) or containers (e.g., as in arrays of test tubes).

Nucleic acids of the marker sets are optionally immobilized, for example by direct or indirect cross-linking, to the solid support. Essentially any solid support capable of withstanding the reagents and conditions used in the particular detection assay can be utilized. For example, functionalized glass, silicon, silicon dioxide, modified silicon, any of a variety of polymers, such as (poly)tetrafluoroethylene, (poly)vinylidenedifluoride, polystyrene, polycarbonate, membranes (e.g., nylon or nitrocellulose), or combinations thereof, can all serve as the substrate for a solid phase array.

In one embodiment, the array is a “chip” composed, e.g., of one of the above specified materials. Polynucleotide probes, e.g., RNA or DNA, such as cDNA, synthetic oligonucleotides, and the like, as discussed above are adhered to the chip in a logically ordered manner, i.e., in an array. Additional details regarding methods for linking nucleic acids and proteins to a chip substrate, can be found in, e.g., U.S. Pat. No. 5,143,854 “Large Scale Photolithographic Solid Phase Synthesis of Polypeptides and Receptor Binding Screening Thereof” to Pirrung et al., issued, Sep. 1, 1992; U.S. Pat. No. 5,837,832 “Arrays of Nucleic Acid Probes on Biological Chips” to Chee et al., issued Nov. 17, 1998; U.S. Pat. No. 6,087,112 “Arrays with Modified Oligonucleotide and Polynucleotide Compositions” to Dale, issued Jul. 11, 2000; U.S. Pat. No. 5,215,882 “Method of Immobilizing Nucleic Acid on a Solid Substrate for Use in Nucleic Acid Hybridization Assays” to Bahl et al., issued Jun. 1, 1993; U.S. Pat. No. 5,707,807 “Molecular Indexing for Expressed Gene Analysis” to Kato, issued Jan. 13, 1998; U.S. Pat. No. 5,807,522 “Methods for Fabricating Microarrays of Biological Samples” to Brown et al., issued Sep. 15, 1998; U.S. Pat. No. 5,958,342 “Jet Droplet Device” to Gamble et al., issued Sep. 28, 1999; U.S. Pat. No. 5,994,076 “Methods of Assaying Differential Expression” to Chenchik et al., issued Nov. 30, 1999; U.S. Pat. No. 6,004,755 “Quantitative Microarray Hybridization Assays” to Wang, issued Dec. 21, 1999; U.S. Pat. No. 6,048,695 “Chemically Modified Nucleic Acids and Method for Coupling Nucleic Acids to Solid Support” to Bradley et al., issued Apr. 11, 2000; U.S. Pat. No. 6,060,240 “Methods for Measuring Relative Amounts of Nucleic Acids in a Complex Mixture and Retrieval of Specific Sequences Therefrom” to Kamb et al., issued May 9, 2000; U.S. Pat. No. 6,090,556 “Method for Quantitatively Determining the Expression of a Gene” to Kato, issued Jul. 18, 2000; and U.S. Pat. No. 6,040,138 “Expression Monitoring by Hybridization to High Density Oligonucleotide Arrays” to Lockhart et al., issued Mar. 21, 2000.

In addition to being able to design, build and use probe arrays using available techniques, one of skill can simply order custom-made arrays and array-reading devices from manufacturers specializing in array manufacture. For example, custom arrays are available through Agilent Technology, Inc. or through Affymetrix Corp., in Santa Clara, Calif. which manufactures DNA VLSIP™ arrays.

In addition to marker sets made up of nucleic acid probes described above, marker sets including polypeptide, peptide, and antibody probes as discussed in the section entitled “Labeled Probes” are favorably used in certain applications. As discussed above for individual probes, sets of probes including multiple members selected from SEQ ID NOs: 31-60, or antibodies specific to such sequences can be used in liquid phase, or immobilized as described above with respect to nucleic acid markers.

Vectors, Promoters and Expression Systems

The present invention includes recombinant constructs incorporating one or more of the nucleic acid sequences described above. Such constructs include a vector, for example, a plasmid, a cosmid, a phage, a virus, a bacterial artificial chromosome (BAC), a yeast artificial chromosome (YAC), etc., into which one or more of the polynucleotide sequences of the invention, e.g., comprising any of SEQ ID NO: 1-30 or SEQ ID NO: 61-403, or a subsequence thereof, has been inserted, in a forward or reverse orientation. For example, the inserted nucleic acid can include a chromosomal sequence or cDNA including a all or part of at least one of SEQ ID NO: 1 through SEQ ID NO: 30, such as a sequence originating on Arabidopsis chromosome 2, or a cDNA corresponding to an mRNA expression product transcribed from a polynucleotide sequence on Arabidopsis chromosome 2. In an embodiment, the construct further comprises regulatory sequences, including, for example, a promoter, operably linked to the sequence. Large numbers of suitable vectors and promoters are known to those of skill in the art, and are commercially available.

The polynucleotides of the present invention can be included in any one of a variety of vectors suitable for generating sense or antisense RNA, and optionally, polypeptide expression products. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, pseudorabies, adenovirus, adeno-associated virus, retroviruses and many others. Any vector that is capable of introducing genetic material into a cell, and, if replication is desired, which is replicable in the relevant host can be used.

In an expression vector, the polynucleotide sequence of interest is physically arranged in proximity and orientation to an appropriate transcription control sequence (promoter, and optionally, one or more enhancers) to direct mRNA synthesis. That is, the polynucleotide sequence of interest is operably linked to an appropriate transcription control sequence. Examples of such promoters include: LTR or SV40 promoter, E. coli lac or trp promoter, phage lambda P_{L} promoter, and other promoters known to control expression of genes in prokaryotic or eukaryotic cells or their viruses. The expression vector also contains a ribosome binding site for translation initiation, and a transcription terminator. The vector optionally includes appropriate sequences for amplifying expression.

For example, constitutive promoters useful in vectors of the invention include the cauliflower mosaic virus (CaMV) 35S transcription initiation region, the 1′- or 2′-promoter derived from T-DNA of Agrobacterium tumefaciens, and other transcription initiation regions from various bacterial, plant or animal genes known to those of skill. Alternatively, the promoter can direct expression of a polynucleotide of the invention in a specific tissue (tissue-specific promoters) or can be otherwise under more precise environmental control (inducible promoters). Examples of tissue-specific promoters under developmental control include promoters that initiate transcription only in certain tissues, such as fruit, seeds, or flowers.

Any of a number of promoters which direct transcription in cells can be suitable. The promoter can be either constitutive or inducible. For example, in addition to the promoters noted above, promoters of bacterial origin which operate in plants include the octopine synthase promoter, the nopaline synthase promoter and other promoters derived from native Ti plasmids. See, Herrara-Estrella et al. (1983), Nature, 303:209-213. Viral promoters include the 35S and 19S RNA promoters of cauliflower mosaic virus. See, Odell et al. (1985) Nature, 313:810-812. Other plant promoters include the ribulose-1,3-bisphosphate carboxylase small subunit promoter and the phaseolin promoter. The promoter sequence from the E8 gene and other genes can also be used. The isolation and sequence of the E8 promoter is described in detail in Deikman and Fischer, (1988) EMBO J. 7:3315-3327. Many other promoters are in current use and can be coupled to an exogenous DNA sequence to direct expression of the nucleic acid.

In addition, the expression vectors optionally comprise one or more selectable marker genes to provide a phenotypic trait for selection of transformed host cells, such as dihydrofolate reductase or neomycin resistance for eukaryotic cell culture, or such as tetracycline or ampicillin resistance in E. coli. The vector comprising the sequences (e.g., promoters or coding regions) from genes encoding expression products and polynucleotides of the invention optionally include a nucleic acid subsequence, a marker gene which confers a selectable, or alternatively, a screenable, phenotype on plant cells. For example, the marker may encode biocide tolerance, particularly antibiotic tolerance, such as tolerance to kanamycin, G418, bleomycin, hygromycin, or in plants: herbicide tolerance, such as tolerance to chlorosluforon, or phosphinothricin (the active ingredient in the herbicides bialaphos or Basta). See, e.g., Padgette et al. (1996) “New weed control opportunities: Development of soybeans with a Round UP Ready™ gene” In: Herbicide-Resistant Crops (Duke, ed.), pp. 53-84, CRC Lewis Publishers, Boca Raton (“Padgette, 1996”). For example, crop selectivity to specific herbicides can be conferred by engineering genes into crops which encode appropriate herbicide metabolizing enzymes from other organisms, such as microbes. See, Vasil (1996) “Phosphinothricin-resistant crops” In: Herbicide-Resistant Crops (Duke, ed.), pp 85-91, CRC Lewis Publishers, Boca Raton) (“Vasil”, 1996).

Additional Expression Elements

Where translation of polypeptide encoded by a nucleic acid comprising a polynucleotide sequence of the invention is desired, additional translation specific initiation signals can improve the efficiency of translation. These signals can include, e.g., an ATG initiation codon and adjacent sequences. In some cases, for example, full-length cDNA molecules or chromosomal segments including a coding sequence incorporating, e.g., a polynucleotide sequence of the invention, a translation initiation codon and associated sequence elements are inserted into the appropriate expression vector simultaneously with the polynucleotide sequence of interest. In such cases, additional translational control signals frequently are not required. However, in cases where only a polypeptide coding sequence, or a portion thereof, is inserted, exogenous translational control signals, including an ATG initiation codon is provided for expression of the relevant sequence. The initiation codon is put in the correct reading frame to ensure transcription of the polynucleotide sequence of interest. Exogenous transcriptional elements and initiation codons can be of various origins, both natural and synthetic. The efficiency of expression can be enhanced by the inclusion of enhancers appropriate to the cell system in use (Scharf D et al. (1994) Results Probl Cell Differ 20:125-62; Bittner et al. (1987) Methods in Enzymol 153:516-544).

Expression Hosts

The present invention also relates to host cells which are transduced with vectors of the invention, and the production of polypeptides of the invention by recombinant techniques. Host cells are genetically engineered (i.e., transduced, transformed or transfected) with a vector, such as an expression vector, of this invention. As described above, the vector can be in the form of a plasmid, a viral particle, a phage, etc. Examples of appropriate expression hosts include: bacterial cells, such as Agrobacterium tumefaciens, E. coli, Streptomyces, and Salmonella typhimurium; fungal cells, such as Saccharomyces cerevisiae, Pichia pastoris, and Neurospora crassa; insect cells such as Drosophila and Spodoptera frugiperda; mammalian cells such as COS, CHO, BHK, HEK 293 or Bowes melanoma; plant cells, etc.

The engineered host cells can be cultured in conventional nutrient media modified as appropriate for activating promoters, selecting transformants, or amplifying the inserted polynucleotide sequences. The culture conditions, such as temperature, pH and the like, are typically those previously used with the host cell selected for expression, and will be apparent to those skilled in the art and in the references cited herein, including, e.g., Freshney (1994) Culture of Animal Cells, a Manual of Basic Technique, third edition, Wiley-Liss, New York and the references cited therein. Expression products corresponding to the nucleic acids of the invention can also be produced in non-animal cells such as plants, yeast, fungi, bacteria and the like. In addition to Sambrook, Berger and Ausubel, details regarding cell culture can be found in Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Atlas and Parks (eds) The Handbook of Microbiological Media (1993) CRC Press, Boca Raton, Fla.

In bacterial systems, a number of expression vectors can be selected depending upon the use intended for the expressed product. For example, when large quantities of a polypeptide or fragments thereof are needed for the production of antibodies, vectors which direct high level expression of fusion proteins that are readily purified are favorably employed. Such vectors include, but are not limited to, multifunctional E. coli cloning and expression vectors such as BLUESCRIPT (Stratagene), in which the coding sequence of interest, e.g., a polynucleotide of the invention as described above, can be ligated into the vector in-frame with sequences for the amino-terminal translation initiating Methionine and the subsequent 7 residues of beta-galactosidase producing a catalytically active beta galactosidase fusion protein; pIN vectors (Van Heeke & Schuster (1989) J Biol Chem 264:5503-5509); pET vectors (Novagen, Madison Wis.); and the like.

Similarly, in the yeast Saccharomyces cerevisiae a number of vectors containing constitutive or inducible promoters such as alpha factor, alcohol oxidase and PGH can be used for production of the desired expression products. For reviews, see Berger, Ausubel, and, e.g., Grant et al. (1987; Methods in Enzymology 153:516-544).

In mammalian host cells, a number expression systems, such as viral-based systems, can be utilized. For example, in cases where an adenovirus is used as an expression vector, a coding sequence is optionally ligated into an adenovirus transcription/translation complex consisting of the late promoter and tripartite leader sequence. Insertion in a nonessential E1 or E3 region of the viral genome will result in a viable virus capable of expressing the polypeptides of interest in infected host cells (Logan and Shenk (1984) Proc Natl Acad Sci 81:3655-3659). In addition, transcription enhancers, such as the rous sarcoma virus (RSV) enhancer, can be used to increase expression in mammalian host cells.

Transformed or transfected host cells containing the expression vectors described above are also a feature of the invention. The host cell can be a eukaryotic cell, such as a mammalian cell, a yeast cell, or a plant cell, or the host cell can be a prokaryotic cell, such as a bacterial cell. Introduction of the construct into the host cell can be effected by calcium phosphate transfection, DEAE-Dextran mediated transfection, electroporation, or other common techniques (Davis, L., Dibner, M., and Battey, I. (1986) Basic Methods in Molecular Biology).

A host cell strain is optionally chosen for its ability to modulate the expression of the inserted sequences or to process the expressed protein in the desired fashion. Such modifications of the protein include, but are not limited to, acetylation, carboxylation, glycosylation, phosphorylation, lipidation, and acylation. Post-translational processing which cleaves a precursor form into a mature form of the protein is sometimes important for correct insertion, folding, and/or function. Different host cells such as bacterial, fungal, plant and animal host cells have specific cellular machinery and characteristic mechanisms for such post-translational activities and can be chosen to ensure the correct modification and processing of the introduced, foreign protein.

For long-term, high-yield production of recombinant proteins encoded by or having subsequences encoded by the polynucleotides of the invention, stable expression systems are typically used. For example, cell lines which stably express a polypeptide of the invention are transfected using expression vectors which contain viral origins of replication or endogenous expression elements and a selectable marker gene. Following the introduction of the vector, cells are allowed to grow for 1-2 days in an enriched media before they are switched to selective media. The purpose of the selectable marker is to confer resistance to selection, and its presence allows growth and recovery of cells which successfully express the introduced sequences. For example, resistant colonies of stably transformed cells can be proliferated using tissue culture techniques appropriate to the cell type.

Host cells transformed with a nucleotide sequence encoding a polypeptide of the invention are optionally cultured under conditions suitable for the expression and recovery of the encoded protein from cell culture. The protein or fragment thereof produced by a recombinant cell can be secreted, membrane-bound, or contained intracellularly, depending on the sequence and/or the vector used.

Plant Transformation

The nucleic acids of the invention can be introduced into plants to modulate growth of the plants. That is, expression of the nucleic acids, e.g., when present as transgenes can modulate growth of the plants. Similarly, transgenic expression of sense or anti-sense sequences of the invention can modulate expression of endogenous forms or homologues of the nucleic acids, thereby modulating growth of the plants. Thus, the sequences specified herein, or homologues (or other variants) thereof, can be expressed to modulate plant growth.

The nucleic acids of the invention are optionally expressed under the control of an inducible promoter, e.g., a promoter regulated by an environmental signal (e.g., a chemical, a hormone (e.g., a plant or insect hormone), heat, light, water or the like. Alternately, a constitutive promoter can be used to drive expression of a nucleic acid of interest.

It can also be useful to stack expression of multiple nucleic acids of the invention in a single plant to modulate growth of the plant, or to stack expression of the nucleic acids of the invention with any other nucleic acid that provides a desired property (resistance to pests, herbicides, etc).

As noted, natural homologues, e.g., of the Arabadopsis sequences noted herein can be identified using standard molecular techniques as noted herein, and/or using sequence comparison methods as noted herein. In one embodiment, nucleic acids corresponding to homologues from a species are introduced as components of expression vectors into plants of that species (e.g., a corn homologue is introduced into corn) to modulate plant growth of the resulting transgenic plant. In another embodiment, nucleic acids from a species are introduced into a different species (e.g., a corn homologue is optionally introduced into a different grass family plant) to modulate plant growth of the resulting transgenic plant.

Accordingly, polynucleotides of the invention can be introduced into an Arabidopsis or any other desired plant genome, e.g., Brassica, Zea, Oryza, Triticum, Hordeum, Lolium, Sorghum, Glycine, Medicago, Helianthus, Lactuca, Beta, Vitis, Solanum, Lycopersicon, Capsicum, Gossypium, Hevea, Linum, Prunus, Citrus, Populus, Pinus, and Quercus, using a number of techniques well established in the art. Methods for transforming a wide variety of higher plant species have been described in the technical and scientific literature (see, e.g., Payne et al. (1992) Plant Cell and Tisue Culture in Liquid Systems John Wiley & Sons, Inc. New York, N.Y.; Gamborg and Phillips (1995) Plant Cell, Tissue and Organ Culture: Fundamental Methods Springer Lab Manual, Springer-Verlag, Berlin; Jones (1995) Plant Gene Transfer and Expression Protocols: Methods in Molecular Biology, Volume 49 Humana Press, Towata, N.J.; and Croy (1993) Plant Molecular Biology Bios Scientific Publishers, Oxfore, U.K., as well as, e.g., Weising et al. (1988) Ann. Rev. Genet. 22:421.

In many cases, introduction of exogenous nucleic acids into a plant genome is facilitated by molecular transformation of plant protoplasts or isolated plant tissues in a tissue culture system, e.g., a liquid tissue culture system, as described in the references above. Numerous protocols for establishment of transformable protoplasts from a variety of plant types and subsequent transformation of the cultured protoplasts are available in the art and are incorporated herein by reference. For examples, see, Hashimoto et al. (1990) Plant Physiol. 93:857; Fowke and Constabel (eds)(1994) Plant Protoplasts; Saunders et al. (1993) Applications of Plant In Vitro Technology Symposium, UPM 16-18; and Lyznik et al. (1991) BioTechniques 10:295, each of which is incorporated herein by reference.

Nucleic acids, e.g., DNA expression vectors comprising the polynucleotides of the invention, can be introduced directly into the genomic DNA of a plant cell using techniques such as electroporation (see, e.g., Fromm et al. (1985) Proc Nat'l Acad Sci USA 82:5824), polyethylene glycol precipitation (see, e.g., Paszkowski et al. (1984) EMBO J. 3:2717) and microinjection of plant cell protoplasts. Ballistic methods, such as DNA particle bombardment can be used to introduce DNA into plant tissues (see, e.g., Klein et al. (1987) Nature 327:70; and Weeks et al. Plant Physiol 102:1077).

Alternatively, the polynucleotides of the invention can be combined with suitable T-DNA flanking regions and introduced into a conventional Agrobacterium tumefaciens host vector. Agrobacterium-mediated transformation is widely used for the transformation of dicots, such as Arabidopsis as well as numerous other species of experimental and commercial interest, as well as certain monocots. For example, Agrobacterium transformation of rice is described by Hiei et al. (1994) Plant J. 6:271; U.S. Pat. No. 5,187,073; U.S. Pat. No. 5,591,616; Li et al. (1991) Science in China 34:54; and Raineri et al. (1990) Bio/Technology 8:33. Transformed maize, barley, triticale and asparagus by Agrobacterium mediated transformation have also been described (Xu et al. (1990) Chinese J Bot 2:81).

Agrobacterium mediated transformation techniques take advantage of the ability of the tumor-inducing (Ti) plasmid of A. tumefaciens to integrate into a plant cell genome, to co-transfer a nucleic acid of interest into a plant cell. Typically, an expression vector is produced wherein the nucleic acid of interest, such as a GAT polynucleotide of the invention, is ligated into an autonomously replicating plasmid which also contains T-DNA sequences. T-DNA sequences typically flank the expression cassette nucleic acid of interest and comprise the integration sequences of the plasmid. In addition to the expression cassette, T-DNA also typically includes a marker sequence, e.g., antibiotic resistance genes. The plasmid with the T-DNA and the expression cassette can then be transfected into Agrobacterium cells. Typically, for effective transformation of plant cells, the A. tumefaciens bacterium also possesses the necessary vir regions on a plasmid, or integrated into its chromosome. For a discussion of Agrobacterium mediated transformation, see, Firoozabady and Kuehnle, (1995) Plant Cell Tissue and Organ Culture Fundamental Methods, Gamborg and Phillips (eds.).

In addition, methods for transforming Arabidopsis in whole plants without tissue culture have been developed, e.g., using vacuum infiltration (Bechtold et al. (1993) “In planta Agrobacterium mediated gene transfer by infiltration of adult Arabidopsis thaliana plants”. CR Acad Sci Paris Life Sci 316:1194-1199) and simple dipping of flowering plants (Desfeux et al. (2000) “Female reproductive tissues are the primary target of Agrobacterium-mediated transformation by the Arabidopsis floral-dip method” Plant Physiol. 123:895-904).

Plant viral vectors can also be used to introduce exogenous nucleic acids comprising the polynucleotides of the invention into a plant genome. Typically, viral vectors are used when transient expression of the exogenous polynucleotide sequence is desirable. Viral vectors are simple to manipulate in vitro and can be easily introduced into mechanically wounded leaves of intact plants of a variety of laboratory plant species as well as common crop species. Over six-hundred-fifty plant viruses have been identified, and both DNA and RNA viruses have been used as vectors for gene replacement, gene insertion, epitope presentation and complementation, (see, e.g., Scholthof, Scholthof and Jackson, (1996) “Plant virus gene vectors for transient expression of foreign proteins in plants,” Annu. Rev. of Phytopathol. 34:299-323). The nucleotide sequences encoding many of these proteins are matters of public knowledge, and accessible through any of a number of databases, e.g. (Genbank: available at the world wide web at ncbi.nlm.nih.gov/genbank/or EMBL: available at the world wide web at ebi.ac.uk.embl/).

Methods for the transformation of plants and plant cells using sequences derived from plant viruses include the direct transformation techniques described above relating to DNA molecules, see e.g., Jones, ed. (1995) Plant Gene Transfer and Expression Protocols, Humana Press, Totowa, N.J., for a recent compilation. In addition viral sequences can be cloned adjacent T-DNA border sequences and introduced via Agrobacterium mediated transformation, or Agroinfection.

Viral particles comprising the plant virus vectors of the invention can also be introduced by mechanical inoculation using techniques well known in the art, (see e.g., Cunningham and Porter, eds. (1997) Methods in Biotechnology, Vol. 3. Recombinant Proteins from Plants: Production and Isolation of Clinically Useful Compounds, for detailed protocols).

Regeneration of Transgenic Plants

Transgenic plant cells which are derived by plant transformation techniques, including those discussed above, can be cultured to regenerate a whole plant which possesses the transformed genotype (e.g., SEQ ID NO: 1-30), and thus the desired phenotype, such as a desirable growth trait. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al. (1983) Protoplasts Isolation and Culture, Handbook of Plant Cell Culture, pp 124-176, Macmillan Publishing Company, New York; and Binding (1985) Regeneration of Plants, Plant Protoplasts pp 21-73, CRC Press, Boca Raton. Regeneration can also be obtained from plant callus, explants, organs, or parts thereof. Such regeneration techniques are described generally in Klee et al. (1987) Ann Rev of Plant Phys 38:467. See also, e.g., Payne and Gamborg, supra. After transformation with Agrobacterium, the explants typically are transferred to selection medium. One of skill will realize that the selection medium depends on the selectable marker that was co-transfected into the explants. After a suitable length of time, transformants will begin to form shoots. After the shoots are about 1-2 cm in length, the shoots should be transferred to a suitable root and shoot medium. Selection pressure should be maintained in the root and shoot medium.

Typically, the transformants will develop roots in about 1-2 weeks and form plantlets. After the plantlets are about 3-5 cm in height, they are placed in sterile soil in fiber pots. Those of skill in the art will realize that different acclimation procedures are used to obtain transformed plants of different species. For example, after developing a root and shoot, cuttings, as well as somatic embryos of transformed plants, are transferred to medium for establishment of plantlets. For a description of selection and regeneration of transformed plants, see, e.g., Dodds and Roberts (1995) Experiments in Plant Tissue Culture, 3^{rd} Ed., Cambridge University Press.

The transgenic plants of this invention can be characterized either genotypically or phenotypically to evaluate the presence of an exogenous nucleic acid, e.g., a polynucleotide of the invention. Genotypic analysis can be performed by any of a number of well-known techniques, including PCR amplification of genomic DNA and hybridization of genomic DNA with specific labeled probes. Phenotypic analysis includes, e.g., survival of plants or plant tissues exposed to a selected biocide or herbicide.

Essentially any plant can be transformed with the polynucleotides of the invention. Suitable plants include agronomically and horticulturally important species. Such species include, but are not restricted to members of the families: Graminae (including corn, rye, triticale, barley, millet, rice, wheat, oats, etc.); Leguminosae (including pea, beans, lentil, peanut, yam bean, cowpeas, velvet beans, soybean, clover, alfalfa, lupine, vetch, lotus, sweet clover, wisteria, and sweetpea); Compositae (the largest family of vascular plants, including at least 1,000 genera, including important commercial crops such as sunflower) and Rosaciae (including raspberry, apricot, almond, peach, rose, etc.), as well as nut plants (including, walnut, pecan, hazelnut, etc.), and forest trees (including Pinus, Quercus, Pseutotsuga, Sequoia, Populus, etc.). The ability to modulate growth of commercially relevant plants using the nucleic acids and proteins of the invention provides a clear utility for such nucleic acids and proteins.

Additional targets for modification by the polynucleotides of the invention, as well as those specified above, include plants from the genera: Agrostis, Allium, Antirrhinum, Apium, Arachis, Asparagus, Atropa, Avena (e.g., oats), Bambusa, Brassica, Bromus, Browaalia, Camellia, Cannabis, Capsicum, Cicer, Chenopodium, Chichorium, Citrus, Coffea, Coix, Cucumis, Curcubita, Cynodon, Dactylis, Datura, Daucus, Digitalis, Dioscorea, Elaeis, Eleusine, Festuca, Fragaria, Geranium, Gossypium, Glycine, Helianthus, Heterocallis, Hevea, Hordeum (e.g., barley), Hyoscyamus, Ipomoea, Lactuca, Lens, Lilium, Linum, Lolium, Lotus, Lycopersicon, Majorana, Malus, Mangifera, Manihot, Medicago, Nemesia, Nicotiana, Onobrychis, Oryza (e.g., rice), Panicum, Pelargonium, Pennisetum (e.g., millet), Petunia, Pisum, Phaseolus, Phleum, Poa, Prunus, Ranunculus, Raphanus, Ribes, Ricinus, Rubus, Saccharum, Salpiglossis, Secale (e.g., rye), Senecio, Setaria, Sinapis, Solanum, sorghum, Stenotaphrum, Theobroma, Trifolium, Trigonella, Triticum (e.g., wheat), Vicia, Vigna, Vitis, Zea (e.g., corn), the Olyreae, the Pharoideae, and many others. As noted, plants in the family Brassicaceae are a particularly favored target plants for the methods of the invention.

Common crop plants which are targets of the present invention include corn, rice, triticale, rye, cotton, soybean, sorghum, wheat, oats, barley, millet, sunflower, canola, peas, beans, lentils, peanuts, yam beans, cowpeas, velvet beans, clover, alfalfa, lupine, vetch, lotus, sweet clover, wisteria, sweetpea, and nut plants (e.g., walnut, pecan, etc).

In cases where expression in the plant chloroplast is desired, the polynucleotide of the invention is modified by the addition of a chloroplast transit sequence peptide to facilitate translocation of the gene products into the chloroplasts. Additionally, methods are available in the art to accomplish transformation directly into the chloroplast accompanied by expression of the transformed polynucleotides (e.g., Daniell et al. (1998) Nature Biotechnology 16:346; O'Neill et al. (1993) The Plant Journal 3:729; Maliga (1993) TIBTECH 11:1). In such cases, it is desirable to employ expression vectors that are designed to specifically to function in the chloroplast. Typically, the coding sequence, e.g., a polynucleotide sequence of the invention, is flanked by two regions of homology to the chloroplastid genome to effect a homologous recombination with the chloroplast genome; often a selectable marker gene is also present within the flanking plastid DNA sequences to facilitate selection of genetically stable transformed chloroplasts in the resultant transplastonic plant cells (see, e.g., Maliga (1993) and Daniell (1998), and references cited therein).

Polypeptide Production and Recovery

Following transduction of a suitable host cell line or strain, and growth of the host cells to an appropriate cell density, the selected promoter is induced by appropriate means (e.g., temperature shift or chemical induction) and cells are cultured for an additional period. The secreted polypeptide product is then recovered from the culture medium. Alternatively, cells can be harvested by centrifugation, disrupted by physical or chemical means, and the resulting crude extract retained for further purification. Eukaryotic or microbial cells employed in expression of proteins can be disrupted by any convenient method, including freeze-thaw cycling, sonication, mechanical disruption, or use of cell lysing agents, or other methods, which are well know to those skilled in the art.

Expressed polypeptides can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxylapatite chromatography, and lectin chromatography. Protein refolding steps can be used, as desired, in completing configuration of the mature protein. Finally, high performance liquid chromatography (HPLC) can be employed in the final purification steps. In addition to the references noted above, a variety of purification methods are well known in the art, including, e.g., those set forth in Sandana (1997) Bioseparation of Proteins, Academic Press, Inc.; and Bollag et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications. Second Edition Wiley-VCH, NY; and Walker (1998) Protein Protocols on CD-ROM Humana Press, NJ.

Alternatively, cell-free transcription/translation systems can be employed to produce polypeptides, e.g., corresponding to SEQ ID NO: 31 through SEQ ID NO: 60, subsequences thereof or sequences or subsequences encoded by the polynucleotides of the invention. A number of suitable in vitro transcription and translation systems are commercially available. A general guide to in vitro transcription and translation protocols is found in Tymms (1995) In vitro Transcription and Translation Protocols: Methods in Molecular Biology Volume 37, Garland Publishing, NY.

In addition, the polypeptides, or subsequences thereof, e.g., subsequences comprising antigenic peptides, can be produced manually or by using an automated system, by direct peptide synthesis using solid-phase techniques (see, Stewart et al. (1969) Solid-Phase Peptide Synthesis, W H Freeman Co, San Francisco; Merrifieid J (i963) J. Am. Chem. Soc. 85:2149-2154). Exemplary automated systems include the Applied Biosystems 431A Peptide Synthesizer (Perkin Elmer, Foster City, Calif.). If desired, subsequences can be chemically synthesized separately, and combined using chemical methods to provide full-length polypeptides.

Conservatively Modified Variations

The polypeptides of the invention include, e.g., those presented in SEQ ID NO: 31 to SEQ ID NO: 60, but also similar polypeptides such as, e.g., homologues, peptides synthesized with modified amino acids, subsequences, peptides with conservative modifications, etc.

For example, the polypeptides of the present invention include conservatively modified variations of SEQ ID NO: 31 to SEQ ID NO: 60. Such conservatively modified variations comprise substitutions, additions, or deletions which alter, add or delete a single amino acid or a small percentage of amino acids (typically less than about 5%, more typically less than about 4%, 2%, or 1%) in any of SEQ ID NO: 31 to SEQ ID NO: 60. Typically, substitutions of amino acids are conservative substitutions according to the six substitution groups set forth in Table 1 (supra).

For example, a conservatively substituted variation of the polypeptide identified herein as SEQ ID NO: 31 will contain “conservative substitutions”, according to the six groups defined above, in up to 17 residues (i.e., 5% of the amino acids) in the 346 amino acid polypeptide.

For example, if four conservative substitutions were localized in the region corresponding to amino acids 2-26 of SEQ ID NO: 31, examples of conservatively substituted variations of this region,

ALKSKLVSL LFLIATLSST FAASFS include:

AMKSKLLSL LFLIAALSST FAASWS and

ALRSKLVSL LFIIATLTST FAASYS and the like, in accordance with the conservative substitutions listed in Table 1 (in the above example, conservative substitutions are underlined). Listing of a protein sequence herein, in conjunction with the above substitution table, provides an express listing of all conservatively substituted proteins.

Finally, the addition of sequences which do not alter the encoded activity of a nucleic acid molecule, such as the addition of a non-functional sequence, provides conservative variations of the basic nucleic acid.

The polypeptides of the invention, including conservatively substituted sequences, can be present as part of larger polypeptide sequences such as occur upon the addition of one or more domains for purification of the protein (e.g., poly his segments, FLAG tag segments, etc.), e.g., where the additional functional domains have little or no effect on the activity of the protein, or where the additional domains can be removed by post synthesis processing steps such as by treatment with a protease.

Modified Amino Acids

Expressed polypeptides of the invention can contain one or more modified amino acid. The presence of modified amino acids can be advantageous in, for example, (a) increasing polypeptide serum half-life, (b) reducing polypeptide antigenicity, (c) increasing polypeptide storage stability. Amino acid(s) are modified, for example, co-translationally or post-translationally during recombinant production (e.g., N-linked glycosylation at N-X-S/T motifs during expression in mammalian cells), or modified by synthetic means (e.g., via PEGylation).

Non-limiting examples of a modified amino acid include a glycosylated amino acid, a sulfated amino acid, a prenlyated (e.g., farnesylated, geranylgeranylated) amino acid, an acetylated amino acid, an acylated amino acid, a PEG-ylated amino acid, a biotinylated amino acid, a carboxylated amino acid, a phosphorylated amino acid, and the like, as well as amino acids modified by conjugation to, e.g., lipid moieties or other organic derivatizing agents. References adequate to guide one of skill in the modification of amino acids are replete throughout the literature. Example protocols are found in Walker (1998) Protein Protocols on CD-ROM Human Press, Towata, N.J.

Antibodies

The polypeptides of the invention can be used to produce antibodies specific for the polypeptides of SEQ ID NO: 31-SEQ ID NO: 60, and conservative variants thereof. Antibodies specific for, e.g., SEQ ID NOs: 31-60, and related variant polypeptides are useful, e.g., for screening and identification purposes, e.g., related to the activity, distribution, and expression of target polypeptides.

Antibodies specific for the polypeptides of the invention can be generated by methods well known in the art. Such antibodies can include, but are not limited to, polyclonal, monoclonal, chimeric, humanized, single chain, Fab fragments and fragments produced by an Fab expression library.

Polypeptides do not require biological activity for antibody production. The full length polypeptide, subsequences, fragments or oligopeptide can be antigenic. Peptides used to induce specific antibodies typically have an amino acid sequence of at least about 10 amino acids, and often at least 15 or 20 amino acids. Short stretches of a polypeptide, e.g., selected from among SEQ ID NO: 31-SEQ ID NO: 60, can be fused with another protein, such as keyhole limpet hemocyanin, and antibody produced against the chimeric molecule.

Numerous methods for producing polyclonal and monoclonal antibodies are known to those of skill in the art, and can be adapted to produce antibodies specific for the polypeptides of the invention, e.g., corresponding to SEQ ID NO: 31-SEQ ID NO: 60. See, e.g., Coligan (1991) Current Protocols in Immunology Wiley/Greene, NY; and Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Goding (1986) Monoclonal Antibodies: Principles and Practice (2d ed.) Academic Press, New York, N.Y.; Fundamental Immunology, e.g., 4^{th} Edition (or later), W. E. Paul (ed.), Raven Press, N.Y. (1998); and Kohler and Milstein (1975) Nature 256: 495-497. Other suitable techniques for antibody preparation include selection of libraries of recombinant antibodies in phage or similar vectors. See, Huse et al. (1989) Science 246: 1275-1281; and Ward, et al. (1989) Nature 341: 544-546. Specific monoclonal and polyclonal antibodies and antisera will usually bind with a K_{D} of at least about 0.1 μM, preferably at least about 0.01 μM or better, and most typically and preferably, 0.001 μM or better.

Defining Polypeptides by Immunoreactivity

The polypeptides of the invention listed in the sequence listing herein, as well as novel variants derived therefrom, which are also encompassed within the present invention, provide a variety of structural features which can be recognized, e.g., in immunological assays. The generation of antisera which specifically binds the polypeptides of the invention, as well as the polypeptides which are bound by such antisera, are a feature of the invention.

The invention includes polypeptides that specifically bind to or that are specifically immunoreactive with an antibody or antisera generated against an immunogen comprising an amino acid sequence, e.g., selected from one or more of SEQ ID NO: 31 to SEQ ID NO: 60. To eliminate cross-reactivity with non related polypeptides, the antibody or antisera can be subtracted with unrelated polypeptides or proteins.

In one typical format, the immunological assay uses a polyclonal antiserum which was raised against one or more polypeptide comprising one or more of the sequences corresponding to one or more polypeptides of the invention, such as SEQ ID NO: 31 to SEQ ID NO: 60, or a subsequence thereof (e.g., a substantial subsequence including at least about 30% of the full length sequence provided). Such an antigenic peptide or polypeptide is referred to as an “immunogenic polypeptide.” The resulting antisera is optionally selected to have low cross-reactivity against unrelated polypeptides, e.g., BSA, and any such cross-reactivity can be removed by immunoabsorbtion with one or more of the unrelated polypeptides, or protein preparations, prior to use of the polyclonal antiserum in the immunoassay.

In order to produce antisera for use in an immunoassay, one or more of the immunogenic polypeptides is produced and purified as described herein. For example, a recombinant protein can be produced in a bacterial host. An inbred strain of mice (used in this assay because results are more reproducible due to the virtual genetic identity of the mice)can be immunized with the immunogenic protein(s) in combination with a standard adjuvant, such as Freund's adjuvant, and a standard mouse immunization protocol (see, Harlow and Lane (1988) Antibodies, A Laboratory Manual, Cold Spring Harbor Publications, New York, for a standard description of antibody generation, immunoassay formats and conditions that can be used to determine specific immunoreactivity). Alternatively, one or more synthetic or recombinant polypeptide derived from the sequences disclosed herein can be conjugated to a carrier protein and used as an immunogen.

Polyclonal sera are collected and titered against the immunogenic polypeptide in an immunoassay, for example, a solid phase immunoassay with one or more of the immunogenic proteins immobilized on a solid support. Polyclonal antisera with a titer of 10⁶ or greater are selected, pooled and subtracted with the control unrelated polypeptides to produce subtracted pooled titered polyclonal antisera.

If desired, the subtracted pooled titered polyclonal antisera are tested for cross reactivity against any unrelated polypeptides. Discriminatory binding conditions are determined for the subtracted titered polyclonal antisera which result in at least about a 5-fold to 10-fold higher signal to noise ratio for binding of the titered polyclonal antisera to the immunogenic polypeptide of interest as compared to binding to the unrelated polypeptide. That is, the stringency of the binding reaction can be adjusted by the addition of non-specific competitors such as albumin or non-fat dry milk, or by adjusting salt conditions, temperature, and/or the like. These binding conditions can be used in subsequent assays for determining whether a test polypeptide is specifically bound by the pooled subtracted polyclonal antisera. In particular, test polypeptides which show at least a 2-5× (i.e., 2-fold to 5-fold) and preferably 10× or higher signal to noise ratio than for the control polypeptides under discriminatory binding conditions, and at least about a half the signal to noise ratio as compared to the immunogenic polypeptide(s) (and typically 90% or more of the signal to noise ratio shown for the immunogenic peptide), shares substantial structural similarity with the immunogenic polypeptide as compared to unrelated polypeptides, and is, therefore, a polypeptide of the invention.

Such methods are also useful for detecting an unknown test protein or polypeptide, which is also specifically bound by the antisera under conditions as described above. In one format, the immunogenic polypeptide(s) are immobilized to a solid support which is exposed to the subtracted pooled antisera. Test proteins are added to the assay to compete for binding to the pooled subtracted antisera. The ability of the test protein(s) to compete for binding to the pooled subtracted antisera as compared to the immobilized protein(s) is compared to the ability of the immunogenic polypeptide(s) added to the assay to compete for binding (the immunogenic polypeptides compete effectively with the immobilized immunogenic polypeptides for binding to the pooled antisera). The percent cross-reactivity for the test proteins is calculated, using standard calculations.

In a parallel assay, the ability of the control proteins to compete for binding to the pooled subtracted antisera is determined as compared to the ability of the immunogenic polypeptide(s) to compete for binding to the antisera. Again, the percent cross-reactivity for the control polypeptides is calculated, using standard calculations. Where the percent cross-reactivity is at least 5-10× as high for the test polypeptides, the test polypeptides are said to specifically bind the pooled subtracted antisera.

In general, the immunoabsorbed and pooled antisera can be used in a competitive binding immunoassay as described herein to compare any test polypeptide to the immunogenic polypeptide(s). In order to make this comparison, the two polypeptides are each assayed at a wide range of concentrations and the amount of each polypeptide required to inhibit 50% of the binding of the subtracted antisera to the immobilized protein is determined using standard techniques. If the amount of the test polypeptide required required to inhibit 50% of the binding of the subtracted antisera to the immobilized protein is less than twice the amount of the immunogenic polypeptide that is required, then the test polypeptide is said to specifically bind to an antibody generated to the immunogenic protein; provided the amount is at least about 5-10× as high as for a control polypeptide.

As an additional determination of specificity, the pooled antisera can be optionally fully immunosorbed with the immunogenic polypeptide(s) (rather than the control polypeptides) until little or no binding of the resulting immunogenic polypeptide subtracted pooled antisera to the immunogenic polypeptide(s) used in the immunosorbtion is detectable. This fully immunosorbed antisera is then tested for reactivity with the test polypeptide. If little or no reactivity is observed (i.e., no more than 2× the signal to noise ratio observed for binding of the fully immunosorbed antisera to the immunogenic polypeptide), then the test polypeptide can be deemed specifically bound by the antisera elicited by the immunogenic protein.

Predicting Plant Growth Traits

The presence of sequences of the invention, or the amount of their expression products, can be predictive of plant growth traits before they actually become apparent. Detection of polynucleotide sequences of the invention in plant cells can predict plant growth traits, such as root length or leaf mass, well before the maturity of a plant. The presence of particular combinations of polynucleotide sequences of the invention can predict one plant growth trait, e.g., large root mass, while a different combination of polynucleotides of the invention can predict another plant growth trait, e.g., short stalk length. In addition, the amount of expression products, such as the quantity of mRNAs transcribed from polynucleotides of the invention, or amount of translated polypeptides of the invention, can be predictive of plant growth traits. The presence of sequences of the invention, combinations of the sequences, and amount of expression products can predict plant growth traits, e.g., in cultured plant cells and immature plants. Such a predictive information can be useful in, e.g., rapid screening of desirable plants in culture or cultivation.

The probes and marker sets of the invention are favorably employed in methods for predicting plant growth traits in an individual specimen, such as cultured plant cells. Nucleic acids of a marker set or individual probes including one or more polynucleotides of the invention, as described, e.g., in the section entitled “Probes,” are hybridized, e.g., as an array, to a DNA or RNA sample from a subject cell or tissue sample. Upon hybridization of the sample to at least a subset of the probes, a signal is detected corresponding to at least one nucleic acid or to expression or activity of an expression product correlatable to a plant growth trait. When expression is detected, the evaluation can be made on a qualitative basis, that is, detecting whether or not an expression product (or multiple expression products) are expressed in a subject cell or tissue sample. Alternatively, the evaluation can be quantitative, to determine whether levels are adequate to provide the desired trait.

While a variety of biological samples reflective of a growth trait can be employed, the specimen is usually selected for ease of acquisition, to minimize invasiveness of the collection procedure to the subject, or to focus on the tissue of interest. Thus, in the context of individual whole plants, individual leaves, roots or branches can be preferred samples, and can be obtained simple cutting. In the case of recombinant inbred lines (RILs) entire individual plants can be sampled knowing they are representative of other available individuals of the line.

For example, a marker set including a plurality (e.g., several or all of SEQ ID NO: 1 through SEQ ID NO: 30 or of SEQ ID NO: 61 through SEQ ID NO: 403) of the polynucleotides of the invention, can be hybridized individually, or as an array, to an RNA or cDNA sample produced, e.g., by a reverse transcription-polymerase chain reaction (RT-PCR), from a subject RNA sample. Typically, prior to hybridization of the probes or array to a subject or “test” specimen, the probe or array is validated and/or calibrated by comparing samples obtained from classes of subjects known to differ with respect to their growth traits. For example, specimens from individuals displaying a high root mass trait are compared to subjects that display low root mass relative to the general population of individual plants. In one embodiment, for example, nucleic acid SEQ ID NO: 397 through SEQ ID NO: 403 have been associated with enhanced root growth in Arabidopsis plants exposed to environments containing either ammonium sulfate or ammonium nitrate fertilizer. See copending provisional application 60/344,499, Identification of Genes Controlling Complex Traits, by Benjamin A. Bowen, et al., filed Dec. 28, 2001.

Alternatively, a marker set including a plurality of antibodies, or other binding proteins, specific for a polypeptide of the invention, e.g., SEQ ID NO: 31-SEQ ID NO: 60, are employed as individual probes or marker sets to evaluate expression of proteins, e.g., corresponding to SEQ ID NO: 31-SEQ ID NO: 60 in a cell or tissue specimen. In this case, rather than, or in addition to, preparing RNA from a sample, proteins are recovered and exposed to the probe or marker set of antibodies, in liquid phase or with either the target of antibody immobilized on a solid substrate, such as a solid phase array.

Patterns of expression that correlate to a particular growth trait are detected by hybridization to one or more probes. In some embodiments, a single probe with a high predictive value is favored, e.g., for ease of handling and cost containment. In other embodiments multiple probes, e.g., the entire marker set, are preferred, e.g., to increase sensitivity or diagnostic or prognostic value. Optimal probes and marker sets are readily ascertained on an empirical basis.

Alternatively, the invention provides an oligonucleotide or polynucleotide probe that detects sequence polymorphisms rather than expression differences between specimens from individuals with different growth traits. Polymorphisms at a nucleotide level can correspond either directly or indirectly to the gene of interest underlying the growth trait, and can be detected in any of several ways, for example, as restriction fragment length polymorphisms, by allele specific hybridization, as amplification length polymorphisms, and the like.

For example, oligonucleotide probes including conservative variants of a polynucleotide sequences can be selected which correspond to polymorphic variations in a target sequence. For example, a probe pair incorporating a single variant nucleotide can be designed to hybridize under allele specific hybridization conditions to allelic target sequences in which one allele is correlated to a fast growth trait and the other allele indicates a relatively slow growth trait. For example, probe sequences are selected from among SEQ ID NO: 1-SEQ ID NO: 30 (or other polynucleotides of the invention) and variants thereof. In some instances, for example, where the cDNA or chromosomal segment has been sequenced and a particular nucleotide polymorphism is associated with a high growth trait, the probes can be chosen to detect the nucleotide polymorphism, e.g., by allele specific hybridization.

Modulating Plant Growth Traits

The invention also provides experimental methods for modulating plant growth traits in vitro and in vivo. Tissue culture and plant models useful for elucidating the molecular mechanisms underlying growth traits as well as for screening and evaluating potential growth control targets are produced by modulating expression or activity of polypeptides (e.g., represented by SEQ ID NO: 31-SEQ ID NO: 60, and conservative variants thereof) encoded by the nucleic acids of the invention.

For example, plant cells in culture can be transfected with a nucleic acid, e.g., comprising a polynucleotide sequence selected from SEQ ID NO: 1 through SEQ ID NO: 30, to produce cells that express a polypeptide involved in plant growth. It will be understood, that where exogenous polynucleotide sequences are introduced into cells, tissues or individual plants, that the polynucleotide sequences can be selected from among SEQ ID NO: 1-30, conservative variants thereof, polynucleotide sequences encoding SEQ ID NO: 31-60, or other homologous polynucleotide sequences such as polynucleotides sequences that hybridize thereto, or polynucleotides that are at least 70%, (or at least about 75%, about 80%, about 85%, about 90%, or at least about 95%) identical thereto. In some cases, it is preferable to link the polynucleotide sequence of interest to the regulatory sequences with which it is typically associated in vivo in nature. Alternatively, in cases where constitutive expression at levels that are in excess of those found in nature is desired, exogenous promoters and enhancers can be employed, as described in detail in the section entitled “Vectors, Promoters and Expression Systems.”

Expression and/or activity of the gene or polypeptide can also be modulated in a negative manner, that is, suppressed. For example, knock out mutations can be produced by homologous recombination of an exogenous gene homologue, e.g., bearing a stop codon, and/or insertion of, e.g., a selectable marker, that disrupts production of an intact transcript. Alternatively, vectors incorporating the sequence of interest in the antisense orientation can be introduced to suppress translation at a post-transcriptional level.

Alternatively, cell lines, e.g., plant or bacterial cells, that express a polypeptide of the invention, e.g., corresponding to one or more of SEQ ID NO: 31-SEQ ID NO: 60, or a subsequence thereof, into which vectors have been transduced that randomly activate expression of associated endogenous sequences upon integration can be isolated. Such vectors have been described, e.g., by Harrington et al. “Creation of genome-wide protein expression libraries using random activation of gene expression.” Nature Biotechnology 19: 440-445, which is incorporated herein by reference. Typically, the vector is constructed with a strong exogenous promoter linked to an exon and an unpaired splice donor site. Upon integration into the genome, splicing with a proximal splice-acceptor site occurs, activating expression of a chimeric transcript encoding at least a portion of the endogenous gene. Cells expressing a polypeptide of interest e.g., SEQ ID NO: 31-SEQ ID NO: 60 can be selected by well known methods, including those based on phenotypic screening methods, antibody or receptor binding, RNA analytical methods, e.g., RT-PCR, northern analysis, MPSS, and the like. By preference, the screening is performed in a high-throughput format.

The above-described methods for producing cell culture or plant cultivation model systems can be adapted for use in the screening of growth modulating environmental factors, e.g., aimed at optimizing application of water, fertilizer or herbicides. For example, it is desirable to select promoters and enhancers that are modulated in response to nutrients or plant hormones.

Following introduction of environmental factors, e.g., application of fertilizers, herbicides, or other molecules that affect plant growth traits, altered expression or activity can be detected at the RNA or protein level. Detection of altered levels of RNA is most conveniently accomplished by such methods as RT-PCR, MPSS, or northern analysis. Protein expression is conveniently monitored using, e.g., antibody based detection methods, such as ELISA'S, immunoprecipitations, or immunohistochemical methods including western analysis. In each of these procedures, the sample including the expressed protein of interest is reacted with an antibody (e.g., monoclonal antibody) or antiserum specific for the protein of interest. Methods for generating specific antibodies are well known and further details are provided above in the section entitled “Antibodies.”

The cell culture models can be used to identify chemical agents capable of favorably regulating the expression or activity of a polypeptide of interest, e.g., a polypeptide selected from among SEQ ID NO: 31-60, in a cell culture system as described above. Most typically, this involves exposing the cells to a chemical or biological composition, e.g., a small organic molecule, or biological macromolecule such as a protein, e.g., an antibody, binding protein, or macromolecular cofactor. Following exposure to the one or more compositions, for example, members of a chemical or biological composition library, such as a combinatorial chemical library, a library of peptide or polypeptide products expressed from a library of nucleic acids, an antibody (or other polypeptide) display library such as a phage display library, etc., modulation of the polypeptide of interest is detected. As discussed above, modulation of the polypeptide can be detected as an alteration in expression at the level of transcription or translation, or as an alteration in the activity of the encoded protein or polypeptide. In some instances, it is desirable to monitor expression or activity of multiple expression products in the same cell, or cell line. The monitored expression products, can be exogenous, i.e., introduced as described above, or endogenous, such as transcripts or polypeptides whose expression or activity is dependent on the amount or activity of a polypeptide of interest.

In cases where the expression or activity of multiple products are of interest, or where the effect of a plurality of different compounds on the expression or activity of one or more expression products, e.g., screening for growth modulating agents as described above, the monitoring assay is conveniently performed in an array. For example, cells can be arrayed by aliquoting into the wells of a multiwell plate, e.g., a 96, 384, 1536, or other convenient format selected according to available equipment. The arrayed cells can exposed to members of a composition library, and the cells sampled and monitored by, e.g., FACS, immunohistochemisty, ELISA, etc. Alternatively, nucleic acids or proteins can be prepared from the arrayed cells, in a manual, semi-automatic or automated procedure, and the products arranged in a liquid or solid phase array for evaluation. Additional details regarding arrays are provided above in the section entitled “Marker Sets.” Alternative high throughput processing methods, such as microfluidic devices, are also available, and can favorably be employed in the context of monitoring modulation of expression products, e.g., corresponding to SEQ ID NO: 1-403.

Typically, when processing and evaluating large numbers of samples, e.g., in a high throughput assay, data relating to expression or activity is recorded in a database, typically the database includes character strings representing the data recorded on a computer or in a computer readable medium.

In addition to tissue culture systems, transgenic plants can be produced which have integrated one or more of the polynucleotide sequences of the invention, e.g., selected from SEQ ID NO: 1 to SEQ ID NO: 30. In this context, commonly used experimental plants include, e.g., Arabidopsis and tobacco.

Such transgenic plant models are useful, in addition to the cultured cells discussed above, for the evaluation of chemical agents suitable for the modulation plant growth traits. Transgenic plant models, e.g., expressing a polypeptide selected from SEQ ID NO: 31-60, are suitable for evaluating fertilizers, hormones and herbicides useful in modulation of plant growth. For example, following administration of a particular herbicide to a transgenic plant expressing a polypeptide of the invention, leaf growth can be monitored. Monitoring can also involve detecting altered expression or activity of an expression product corresponding to one or more of SEQ ID NO: 1-403 as discussed above.

Kits and Reagents

Certain embodiments of the present invention can be optionally provided to a user as a kit. For example, a kit of the invention can contain one or more nucleic acid, polypeptide, antibody, and/or cell line described herein. Most often, the kit contains a diagnostic nucleic acid or polypeptide, e.g., antibody, probe set, e.g., as a cDNA microarray packaged in a suitable container, or other nucleic acid such as one or more expression vector. The kit typically further comprises, one or more additional reagents, e.g., substrates, labels, primers, for labeling expression products, tubes and/or other accessories, reagents for collecting samples, buffers, hybridization chambers, cover slips, etc. The kit optionally further comprises an instruction set or user manual detailing preferred methods of using the kit components for discovery or application of gene sets. When used according to the instructions, the kit can be used, e.g., for evaluating expression or polymorphisms in a plant sample, e.g., for evaluating growth traits.

Digital Systems

The present invention provides digital systems, e.g., computers, computer readable media, and integrated systems, comprising character strings corresponding to the sequence information herein for the polypeptides and nucleic acids herein, including, e.g., those sequences listed herein and the various silent substitutions and conservative variations thereof. Integrated systems can further include, e.g., gene synthesis equipment for making genes corresponding to the character strings.

Various methods known in the art can be used to detect homology or similarity between different character strings, or can be used to perform other desirable functions such as to control output files, provide the basis for making presentations of information including the sequences, and the like. Examples include BLAST, discussed supra. Computer systems of the invention can include such programs, e.g., in conjunction with one or more data file or data base comprising a sequence as noted herein.

Thus, different types of homology and similarity of various stringency and length can be detected and recognized in the integrated systems herein. For example, many homology determination methods have been designed for comparative analysis of sequences of biopolymers, for spell-checking in word processing, and for data retrieval from various databases. With an understanding of double-helix pair-wise complement interactions among 4 principal nucleobases in natural polynucleotides, models that simulate annealing of complementary homologous polynucleotide strings can also be used as a foundation of sequence alignment or other operations typically performed on the character strings corresponding to the sequences herein (e.g., word-processing manipulations, construction of figures comprising sequence or subsequence character strings, output tables, etc.).

Thus, standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™ or Paradox™) can be adapted to the present invention by inputting a character string corresponding to one or more polynucleotides and polypeptides of the invention (either nucleic acids or proteins, or both). For example, a system of the invention can include the foregoing software having the appropriate character string information, e.g., used in conjunction with a user interface (e.g., a GUI in a standard operating system such as a Windows, Macintosh or LINUX system) to manipulate strings of characters corresponding to the sequences herein. As noted, specialized alignment programs such as BLAST can also be incorporated into the systems of the invention for alignment of nucleic acids or proteins (or corresponding character strings).

Systems in the present invention typically include a digital computer with data sets entered into the software system comprising any of the sequences herein. The computer can be, e.g., a PC (Intel x86 or Pentium chip-compatible DOS™, OS2™ WINDOWS™ WINDOWS NT™, WINDOWS95™, WINDOWS98™ LINUX based machine, a MACINTOSH™, Power PC, or a UNIX based (e.g., SUN™ work station) machine) or other commercially common computer which is known to one of skill. Software for aligning or otherwise manipulating sequences is available, or can easily be constructed by one of skill using a standard programming language such as Visualbasic, Fortran, Basic, Java, or the like.

Any controller or computer optionally includes a monitor which is often a cathode ray tube (“CRT”) display, a flat panel display (e.g., active matrix liquid crystal display, liquid crystal display), or others. Computer circuitry is often placed in a box which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user and for user selection of sequences to be compared or otherwise manipulated in the relevant computer system.

The computer typically includes appropriate software for receiving user instructions, either in the form of user input into a set parameter fields, e.g., in a GUI, or in the form of preprogrammed instructions, e.g., preprogrammed for a variety of different specific operations. The software then converts these instructions to appropriate language for instructing the operation of the fluid direction and transport controller to carry out the desired operation.

The software can also include output elements for controlling nucleic acid synthesis (e.g., based upon a sequence or an alignment of a sequences herein) or other operations.

General Molecular Techniques

In the context of the invention, nucleic acids and/or proteins are manipulated according to well known molecular biology methods. Detailed protocols for numerous such procedures are described in, e.g., in Ausubel et al. Current Protocols in Molecular Biology (supplemented through 2000) John Wiley & Sons, New York (“Ausubel”); Sambrook et al. Molecular Cloning—A Laboratory Manual (2nd Ed.), Vol. 1-3, Cold Spring Harbor Laboratory, Cold Spring Harbor, N.Y., 1989 (“Sambrook”), and Berger and Kimmel Guide to Molecular Cloning Techniques, Methods in Enzymology volume 152 Academic Press, Inc., San Diego, Calif. (“Berger”).

In addition to the above references, protocols for in vitro amplification techniques, such as the polymerase chain reaction (PCR), the ligase chain reaction (LCR), Qβ-replicase amplification, and other RNA polymerase mediated techniques (e.g., NASBA), useful e.g., for amplifying cDNA probes of the invention, are found in Mullis et al. (1987) U.S. Pat. No. 4,683,202; PCR Protocols A Guide to Methods and Applications (Innis et al. eds) Academic Press Inc. San Diego, Calif. (1990) (“Innis”); Arnheim and Levinson (1990) C&EN 36; The Journal Of NIH Research (1991) 3:81; Kwoh et al. (1989) Proc Natl Acad Sci USA 86, 1173; Guatelli et al. (1990) Proc Natl Acad Sci USA 87:1874; Lomell et al. (1989) J Clin Chem 35:1826; Landegren et al. (1988) Science 241:1077; Van Brunt (1990) Biotechnology 8:291; Wu and Wallace (1989) Gene 4: 560; Barringer et al. (1990) Gene 89:117, and Sooknanan and Malek (1995) Biotechnology 13:563. Additional methods, useful for cloning nucleic acids in the context of the present invention, include Wallace et al. U.S. Pat. No. 5,426,039. Improved methods of amplifying large nucleic acids by PCR are summarized in Cheng et al. (1994) Nature 369:684 and the references therein.

Certain polynucleotides of the invention, e.g., SEQ ID NO: 61-SEQ ID NO: 403, can be synthesized utilizing various solid-phase strategies involving mononucleotide- and/or trinucleotide-based phosphoramidite coupling chemistry. For example, nucleic acid sequences can be synthesized by the sequential addition of activated monomers and/or trimers to an elongating polynucleotide chain. See e.g., Caruthers, M. H. et al. (1992) Meth Enzymol 211:3. In lieu of synthesizing the desired sequences, essentially any nucleic acid can be custom ordered from any of a variety of commercial sources, such as The Midland Certified Reagent Company (mcrc@oligos.com), The Great American Gene Company (www.genco.com), ExpressGen, Inc. (www.expressgen.com), Operon Technologies, Inc. (www.operon.com), and many others.

Similarly, commercial sources for nucleic acid and protein microarrays are available, and include, e.g., Affymetrix, Santa Clara, Calif. (http://www.affymetrix.com/); and Agilent, Palo Alto, Calif. (http://www.agilent.com) Zyomyx, Hayward, Calif. (http://www.zyomyx.com); and Ciphergen Biosciences, Fremont, Calif. (http://www.ciphergen.com/).

### EXAMPLES

The following examples are offered to illustrate, but not to limit, the claimed invention.

### Example 1

### Growth Gene Combinations in Different Environments

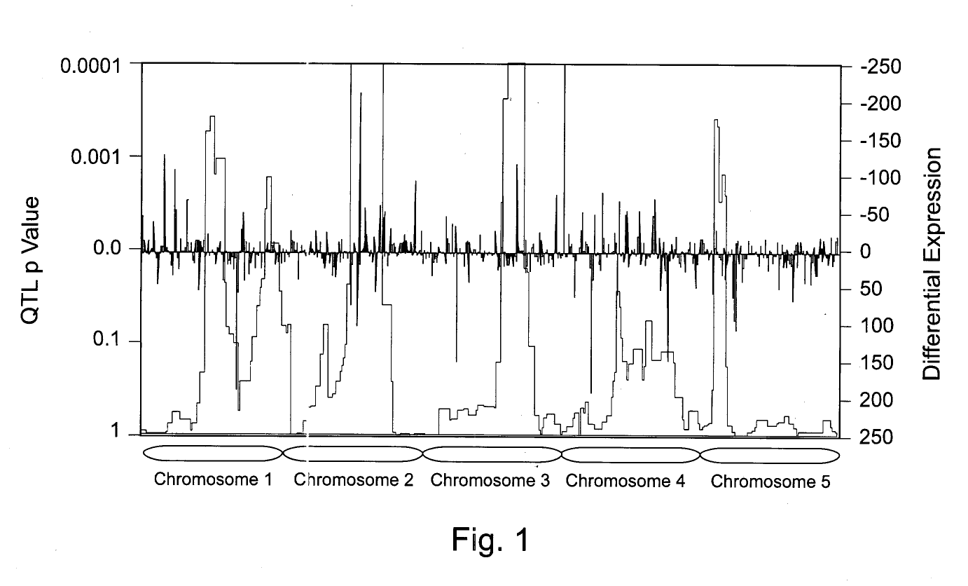

Genes associated with a particular plant growth trait, such as root length, can vary depending on the environment in which the plant is grown. For example, as described in “Identification of Fenes Controlling Compex Traits” by Benjamin A. Bowen, et al., filed Dec. 28, 2001 (Attorney Docket No. 37-000800US) incorporated herein by reference, gene expression by massively parallel signature sequence (MPSS) analysis was determined for Arabidopsis plants having long roots and short roots in ammonium nitrate fertilizer. FIG. 1 shows differential gene expression between the plants having long and short roots. Similar analysis was carried out comparing gene expression in long root and short root Arabidopsis plants but grown in ammonium sulfate fertilizer. In the ammonium nitrate environment, 56 genes were found to have differential expression between long and short root plants and also to be correlated to root growth by quantitative trait locus (QTL) analysis. In the ammonium sulfate environment. 80 genes were found to have differential expression between long and short root plants and also to be correlated to root growth by QTL analysis. Only 7 genes were found to be correlated in the same direction in both environments. The combination of genes associated with root length was considerably different depending on the nutritional environment. Sequences of the present invention are similarly expressed in unique combinations depending on environmental factors.

### Example 2

### Genes Associated with Different Plant Growth Traits

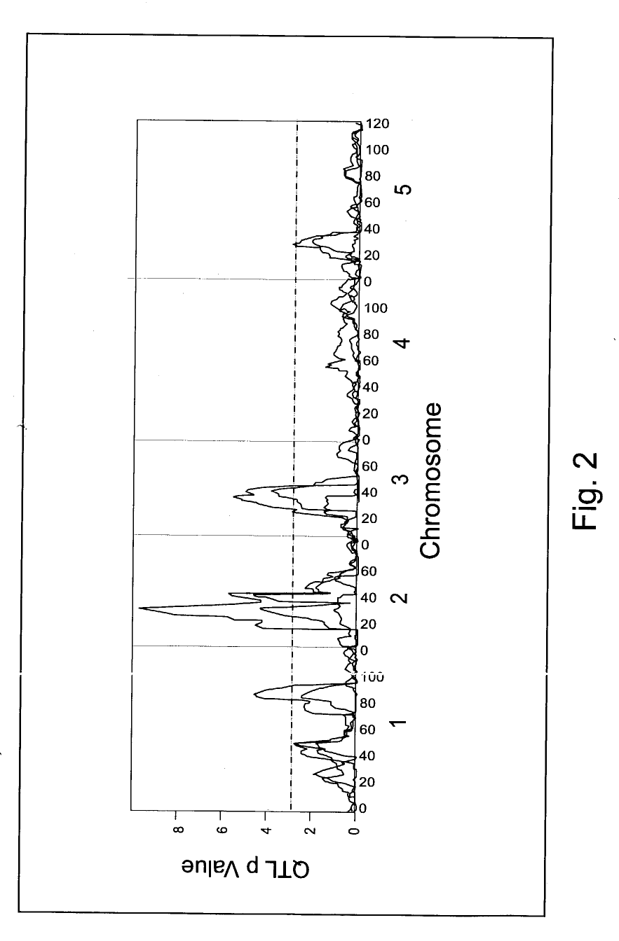

The combination of genes associated with one plant growth trait, such as root length, is often different from the combination of genes associated with another growth trait, such as aerial mass. FIG. 2 shows Arabidopsis QTL plots for three plant growth traits (root length, aerial mass, and root mass). Although there is some overlap of the plots for each trait, QTL analysis would identify a unique combination of differentially expressed genes associated with each trait. For example, differential expression analyses were carried out on long root and short root plants grown with ammonium nitrate fertilizer. Forty-six genes were found to have differential expression between long and short root plants and also to be correlated to root growth by quantitative trait locus (QTL) analysis. The combination of sequences of the present invention also varies uniquely with different plant growth traits.

While the foregoing invention has been described in some detail for purposes of clarity and understanding, it will be clear to one skilled in the art from a reading of this disclosure that various changes in form and detail can be made without departing from the true scope of the invention. For example, the sequences, techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

Sequence ID Table:

| | |
|---|---|
| SEQ ID NO. | SEQ |

| | |
|---|---|
| 1 | cacaaatcct aacgccaata gtatagattc aattagaatt aaaaccgatc caagtataga |
| | ttgattcaat tagaatatgg aattcaaaga gaagattatt gatggactta cacttatcgc |
| | aaaccatctt cttcttccga gagagaaata tgaagaaacc ctaacgccta aatcaattcg |
| | aatgggttag agttacgacg aaaacttatc ggtgttgaaa tttttatcta tgtttaaata |
| | tatttttttt ccttttctgg atttggaaag tcggatatgt ctcgtcaaaa ctcatagcct |
| | cacaggtatt ttatgccacg aatcgtaata atccacgtgg tacatcaacc aataaaaacg |
| | ttccacgtgg tacaaccagc gagataccaa gaacttcgag accttcttct ccagatagag |
| | gctttccggt aaacggcaaa tacccttttc cttcactttc ttcgtcttct cgaatctgag |
| | agaacgagag atcaacaaca ATGGCGCTCA AATCAAAACT CGTCTCTCTT CTCTTCCTCA |
| | TAGCAACACT ATCATCCACA TTCGCAGCTT CGTTTTCCGA TTCGGATTCC GATTCAGATC |
| | TTCTCAACGA ACTTGTATCT CTCAGATCAA CAAGCGAATC AGGCGTAATC CATCTCGATG |
| | ACCATGGAAT CTCAAAATTC CTAACCTCCG CTTCCACGCC TCGTCCTTAC TCGTTACTCG |
| | TCTTCTTCGA CGCTACTCAA CTCCACAGCA AAAACGAGCT TCGTCTTCAA GAGCTCCGTC |
| | GCGAATTCGG CATCGTCTCC GCTTCATTCC TCGCTAACAA CAATGGATCT GAAGGAACTA |
| | AGCTTTTCTT CTGTGAGATC GAGTTTTCGA AGTCTCAATC TTCGTTCCAG CTCTTTGGCG |
| | TTAACGCTTT ACCTCACATT CGTCTTGTAA GTCCTTCGAT ATCGAATCTA CGTGATGAAT |
| | CTGGTCAAAT GGATCAATCG GATTACTCTA GATTAGCTGA ATCAATGGCT GAGTTTGTTG |
| | AGCAACGAAC TAAACTCAAG GTCGGTCCTA TTCAACGTCC ACCGCTACTT TCGAAACCAC |
| | AGATCGGTAT TATCGTTGCG TTGATCGTTA TCGCTACTCC GTTTATCATC AAAAGAGTTT |
| | TGAAAGGAGA AACTATTCTT CATGATACTA GACTTTGGTT ATCTGGTGCT ATCTTCATTT |
| | ACTTCTTTAG TGTTGCTGGT ACAATGCACA ACATTATCAG GAAAATGCCG ATGTTTCTTC |
| | AAGATCGTAA CGATCCGAAT AAGCTTGTGT TTTTCTACCA AGGATCTGGA ATGCAGCTTG |
| | GAGCTGAAGG ATTTGCTGTT GGATTCTTGT ATACTGTTGT TGGATTGCTT TTGGCGTTTG |
| | TTACCAATGT GCTTGTTCGA GTGAAGAATA TTACTGCACA AAGGTTGATT ATGCTTTTGG |
| | CTTTGTTCAT ATCGTTCTGG GCTGTGAAGA AAGTTGTTTA CTTGGATAAC TGGAAGACTG |
| | GATATGGAAT TCATCCGTAT TGGCCATCGA GTTGGCGTTG Attacatcac acttgaggat |
| | ctctgtttca caaggtaatg gctttagttt tggaaaaaca gttatgggaa ttgagtaatg |
| | atgtttctgg atgttttgtg tttcgatttg aaatactttt gaatcggtgt agtactacta |
| | tttcagatgg tttaaaactc cttactgtta cattagtcca ttgttaagtt atttatctga |
| | atgagtaact tatataacca agaatatggg atctttagtc gattgaatat aggaaccata |
| | tttggaaatt caggtactgt ttcttgagat cagtctagga ttgttgttat ttggtacatt |
| | gacactttta gagtttctat gtgtcttcag ccttgcgccc cttgcttact gcatctattc |
| | agaaaaaggg actttgtgat tgaggatagt gtttctgttt aagcattatg ggaccttatg |
| | ttttgtcgtt gactgtgtcc tcttctcgtt ttgctctctg ttttagaatg agtctaagta |
| | a |
| 2 | atttaaatgt gttataatat ttgataaaaa atttgaatct ttttaaaaat atatataatt |
| | gtgttaaaaa aaactatact ttttattatt ttattttatc ttcctttaaa atgttaaatt |
| | taaatttatt ttcaaaaaat ttgataattt taggcttttt gataatgttt ttcaactttt |
| | tatataatat ataagtacat attgttttat tctaaaatcg tttagatctt aacgaatagt |
| | tataggcgtt agacggcctc aactaattgt tataagtgtt agacggaaag ttaccgtccc |
| | cttagcgttt attttaacat taaaagaaaa gatacatact attaaactaa tggagtatta |
| | acaagaaaaa aaagaaagag taaaatacga aaggttcctt aagcaagttt ataaatattt |
| | atagccaaaa acaaaagcaa aaccaaaaat cacaagtaac cccaaaagaa aaaaagcaaa |
| | gagagaggaa aagaaaaaaa ATGACGAAGA CGATGATGAT CTTTGCGGCG GCGATGACGG |
| | TGATGGCTTT GCTTTTGGTT CCGACTATTG AAGCACAAAC TGAGTGCGTG AGCAAGCTAG |
| | TCCCTTGCTT CAACGACCTG AACACGACAA CAACGCCGGT GAAAGAATGT TGCGACTCGA |
| | TTAAAGAAGC GGTGGAGAAG GAACTTACAT GTCTCTGTAC AATCTACACC AGTCCAGGTT |
| | TGCTCGCTCA GTTCAACGTC ACCACTGAGA AAGCTCTCGG TCTTAGCCGT CGTTGCAACG |
| | TCACCACTGA TCTCTCCGCT TGTACCGgta accaatttca ttttctccga tctccgattt |
| | tttaattttt ttgtcaacaa catgcattat gaatggattt gtggattctg attaatgtga |
| | atgtgactaa gaaaattagc atagtttttt gtctactgct aacatttttt agatcttgtt |
| | gagattatga aacagagatt tgcaatttca tatatcagta ttaatcatgt ttttgttttt |
| | tgtttagCTA AAGGAGCTCC ATCGCCAAAA GCTTCTTTAC CTCCTCCAGC TCCAGgtatg |
| | aaccaaactc ttcacctact ccttacaatt atttccttga atactttgtt atcaaaaaaa |
| | aaaaaaaatg aaatattgat cgacttgatt gtgtattaat tgaattattc gattgatttg |
| | attagtagag ttaattaacc aaatcaaatg gtgttaatca aggcaattat tcaattgata |
| | ctctaaatcg atcttataat tttcccagat ttttctctct ttttttgttt tctatataaa |
| | aaacataaac agagtgtgaa tgccagcttt tacttgtgta ctttattttg tctcgagtat |
| | tgacttgaat aattcggaca aaaccactaa aaaatgaaac ttgtcagatt ttttattttt |
| | ttataaattt tttatttgtt atttgctgat tgacgatttg tcttatatta tatggatggg |
| | tttctaaata ttcagCAGGG AATACCAAAA AAGACGCCGG AGCTGGGAAC AAGCTCGCCG |
| | GTTATGGAGT CACCACCGTG ATCTTGTCTT TGATCTCATC CATCTTCTTC TGAattcctt |
| | tacccggttt tattattatt agctcaataa attctcgaga tttgtttgct tttggcttaa |
| | cttatttaat atttaaagaa aaacaaaaag tattttttgt tcacatgtta tgtattatca |
| | ttgattcatt attgagtccc atgttagtat atttaccggt tataatcgga ctctatcatt |
| | tgcatatctg atttgagtgt ggatctgtgt tgttaattga tgtaatcttt attatataaa |
| | ttgaaaatga aaacaaaata taaaaaactg tgttggttta aaggtcccaa tcctcatttt |
| | ggtaggtttg actaccaact agaaacaata tcatccataa tattgcttct ttgtgctatc |
| | ttattaaatg taaaccaaga acgcagtttt attctctaat tgtgttcata aattaaacaa |
| | caaaagaaca gaatcgcaaa tttaattagg cgatgcgagt aacaacagca tgtatagcat |
| | cagcgagttg agg |
| 3 | ctgtatatga cttatcacca tgagattgta ataactctta tctaataata ctcactcaag |
| | taaaagatcc aataatcttc aaacgaaagt agtaccaggt atgaaactcc agcgttgatg |
| | atgtgagctt ctcaatatct actagtcaaa gacgcatcgg atcgatcatc ggagttgcat |
| | cggaatttat cgggaaagaa tggattgggc ccaatgtgga aatgataagt cgtatgggcc |
| | taaatcattt agtcgtaggc ccaatatgag tttaagctct ttgatatttc agagaatgtt |
| | attcaattta ttagtaattt tcaaatgata taaattcaat ttattaatca cttggttaaa |
| | acttatacac gtgaaaaaat gagaaatcat tttagtacat tgttgaccat ctttttcgta |
| | tagactacta tctctgatct cttgcgagtt aagtcagtaa ctaggaaaat tcagaagcgc |
| | tctcaatctc aaaaatatcc ATGGCGGCGA TTACAGAATT TCTACCAAAA GAGTACGGAT |
| | ATGTCGTTCT CGTCCTCGTC TTCTACTGTT TCCTCAACCT CTGGATGGGT GCTCAAGTCG |
| | GCAGAGCTCG CAAAAGgttt ccacgaaact cctagatcgt taacgcttga attgccgtga |
| | tttcgccact aaaatcgaat cgaggacgat gctagatcgt tccctttgtt cttgattgga |
| | atcgaatttt aactgaaatc tgtagattga tgtgacctaa aactagaatt ttgcaatttt |
| | cgtcctaagt ttttggattc tgtagtctga ttcattgttt tgatgttatc atcagttcga |
| | tttcaagttt attgaactta cgatttcaat ctgttgtttg tttgttcatc ttctactaat |
| | tgattagtat gagcgagatt gtcttatcgg ttagatctgt tgtttgttca tcttcaattt |
| | tgaatgatct cacatgagtc tatgatcttg atgcagGTAC AACGTCCCGT ATCCAACTCT |
| | ATATGCAATA GAATCAGAAA ACAAAGATGC TAAGCTCTTC AACTGTGTTC AGgtttgaaa |
| | tatagttaaa acaatacttg tgtgattctg ttttcttgta ctacttgtta ttgagatgtg |
| | ataaaatttg tggttgtagA GAGGACATCA AAACTCTTTA GAGATGATGC CAATGTATTT |
| | CATACTGATG ATCCTCGGTG GGATGAAGCA CCCTTGTATC TGTACTGGCC TTGGTTTGCT |
| | TTACAACGTT AGCCGATTCT TCTACTTTAA AGGTTATGCT ACTGGAGATC CCATGAAGCG |
| | TCTTACGATC GGgtttgttc ttttatcctc ttatcagtgt tcattatctt tattgattga |
| | tttagttatg ttagtcaata ggatatagag tttagacttg tatataaggt tgtaacttgc |
| | aagtatagtt tcattaactg atttcttcgg ttattgtatc aaagcattga tctaaggctc |
| | taagctcaac cattttccgt tttgcgtatc aaatgtttct cgctttcttt gtctttgatt |
| | cttgggaaat ttctttgttt ctgcatacag cttttcccat tcttcgtttc tttactcggt |
| | tctgtattta ctacgacttt gttccacgtc ttcgtctcta aatcgagttt acgtagataa |
| | tcgttgtaat ctacaatgtt gcagttaagt tagtcagagt aatagttaag agttaagact |
| | tgtacatacg gttgtaagtg aacattttcc taaactgact tcttctgtta tggtgtcaga |
| | gcgtgaagct aagctcaacg atttcttcgt gtttctgata agtaacaagc caccaaagtc |
| | tgattactta tctttctaat ctataatgtt gcagGAAATA CGGTTTCTTG GGGTTGCTAG |
| | GTCTGATGAT ATGTACCATC TCGTTTGGTG TCACTCTGAT CCTTGCTTGA gctactcgtt |
| | tctggggtta atgattctct ggtttgctcg aagaatatag aaccaatgct tgtaagctgt |
| | ccacaaaact tgtgtaatac tttagagttt gtcactttta aaagtttgta ataaatcatg |
| | gcttcataga acagttgaaa tttcacatcc gtagacgtta ataaagattt gaattatgaa |
| | gacactttct ggttatttta taattccatc tatctatatc tctgtactga agtgatcaaa |
| | acacttacga cacgttatct tggcttgtta ctcaaaaaat gaaaaaaata aactaaaaac |
| | gtgaacggca ggattcgaac ctgcgcgggc aaagcccaca tgatttctag tcatgcccga |
| | taaccactcc ggcacgtcca ctgtttgaga tgtaacttaa atattaagat aatataatta |
| | taaataaaga caacacgtta cgatactacg tggatagtaa ctaactattt gctgaattat |
| | gataaagtcg |
| 4 | ttcaacttta cctatcagtt tgttggatca atttattacc atccaattct cttgttatta |
| | ttcaaagttc aaacattccg ttccaatgtt aactttgtaa agtagtaaat ggtaagtaac |
| | aataactcta aatacctacc cttacaaatt aaaaattcaa cgcctacata aattatctac |
| | ctactagaat ttaaatatat aaaatcctag aataagtcaa caatcatatt aatgactaaa |
| | aattaccaaa actaaattat ttcattagtt taaaaaaaaa acaatttatt atattttata |
| | taatattata atgtttgcaa aaacagagta tcacgtcacc ttctctctct ctctatctct |
| | gtatcctctc attgcactat aagtactacc acaaccacga actctaaagc atcatctcat |
| | taacaaaaat aaaacacaca atctcaagat tttctacttc ttattacaaa gattcaatct |
| | tcttgtttct tcttgcaacc ATGAGTCTTC TTGCAGATCT TGTTAACCTT GACATCTCAG |
| | ACAACAGTGA AAAGATCATC GCTGAATACA TATGgttcgt cttcttcctc tgcttttgac |
| | catttgagtt tctctggttt tttctgttct tatcggaaaa caagagcttg agttaaagat |
| | ttgaatctta aagtcaatct tatcttaaag tcaatctttg tcatttacca ttttgtatta |
| | catctctaat ttggttttaa ttcaaatagG GTTGGTGGTT CTGGTATGGA CATGAGAAGC |
| | AAAGCCAGGg taatttaatc tttctttaac tataatttct ttgacaaatt gtaacttttc |
| | tcggagagat ttgattcgat tgaattacta agactctggt ttgttgcctg cagACTCTCC |
| | CTGGACCTGT GACCGATCCA TCAAAACTTC CAAAGTGGAA CTATGATGGT TCAAGCACTG |
| | GTCAAGCTCC TGGTCAAGAC AGTGAAGTGA TCTTATAgta agtctcttca agattaaaac |
| | caaaaaaaaa agtctcttca agattttctc taaagatcca tctcttttgt tttttgttta |
| | ctttcttaat aatatttgtt gtatttgtgt ttcttagCCC TCAAGCAATT TTCAAAGATC |
| | CATTCCGTAG AGGCAACAAC ATCCTTgtga gtttaaactt tttttttttt tttcttgcta |
| | tatgttctgt ttttagcggt taaagattaa cgttttttat cggtttgatc agGTTATGTG |
| | TGATGCTTAC ACTCCAGCGG GAGAGCCAAT CCCTACTAAC AAGCGACATG CTGCGGCTGA |
| | GATCTTTGCT AACCCTGATG TTATTGCTGA AGTGCCATGg ttaatccaaa ttcccctgtt |
| | ctttttatat agctttttcg ctttcttgcg gtggtcgtag atcgctgatt ttttttccgg |
| | ttaattagGT ATGGAATCGA ACAAGAATAC ACTTTGTTGC AGAAGGATGT GAACTGGCCT |
| | CTTGGATGGC CCATTGGTGG CTTCCCTGGC CCTCAGgtac attccgtttt tgcggagttt |
| | tttcgtttgt ttactgctct ttttcgattc tccgttcttg gcttctgaat tatctcttgc |
| | actcttgcag GGACCATACT ACTGCAGTAT TGGAGCTGAC AAATCTTTTG GAAGAGACAT |
| | TGTTGATGCT CACTACAAAG CCTCTTTGTA TGCTGGAATC AACATCAGTG GGATCAATGG |
| | AGAAGTCATG CCGGGACAAT GGGAGTTCCA AGTCGGCCCA TCGGTCGGTA TCTCAGCTGC |
| | TGATGAAATA TGGATCGCTC GTTACATTTT GGAGgtataa tttaaaacca ttcacttttc |
| | gattcttgtt gatctcttta aggaaatata aacttataac acaagttttg gtggttttaa |
| | aaacagAGGA TCACAGAGAT TGCTGGTGTG GTTGTATCTT TTGACCCAAA ACCTATTCCT |
| | GGTGACTGGA ATGGAGCTGG TGCTCACACC AATTACAGgt aaaaagaatc atgaatcttt |
| | tctcttgtta gatcattaca atgtttgtga gaacattcaa gaaaatggtg aacgttttta |
| | tttcagTACT AAATCAATGA GGGAAGAAGG AGGATACGAG ATAATCAAGA AGGCGATCGA |
| | GAAGCTTGGC TTGAGACACA AGGAACACAT TTCCGCTTAC GGTGAAGGAA ACGAGCGTCG |
| | TCTCACGGGA CACCATGAAA CTGCTGACAT CAACACTTTC CTTTGGgtaa agattttaga |
| | acattgtttt atttgtaaaa tgtttgataa cattttctga tctttgtgtt tgaatcttct |
| | ttaaaaagGG TGTTGCGAAC CGTGGTGCAT CGATCCGAGT AGGACGTGAC ACCGAGAAAG |
| | AAGGGAAGGG ATACTTTGAG GATAGGAGGC CAGCTTCAAA CATGGACCCT TACGTTGTTA |
| | CTTCCATGAT TGCAGAGACT ACACTCCTCT GGAACCCTTG Aaaggatgat ccgtaactct |
| | tgaagttgct tctgattggg ttttttggaa gttccaagct tgtcttttct ctacagtgtg |
| | tattaagcaa ttgtaccggt tgacactgcc ggagtttgtg atttggggcc tttctttctt |
| | tttcttcttt ttataatctt ttgggttctg tggttagagc aaattcggtt tgctctgttt |
| | gtttgacctt tattgaaacc tttggtattg gtactaataa tacaatctga aaaggcctct |
| | tcatgtttca atgttagaga ctaattaaag atctctttta tttttcattt tatacaaaca |
| | tgaaacacca atgttgatcc tgtctggtcc gtttttgatc tatgactcac aagatcgttg |
| | cgtactcata tcaacggctt tttgaacccc tttgtttgca aacaaaccac caatgtggga |
| | tgcttatcag tagaccgaac aaatgactac ttctccggaa ttttatttcc tttcaccttc |
| | c |
| 5 | taggactttt actatggtaa atcggtttag cacaatacac atgactttat gttattcatt |
| | cttcattcgt atatggataa aaaatcagcg atgctaaaca gatctcaata tgtatgtgaa |
| | cttgtgaagt agcaaattgt tgcttattcc actatattaa gtcaagtttc cacaatgtgc |
| | cagacaatcc ctagttgttt agattccaag atttcgacaa tgtaacaccc gttaataatt |
| | cacaacagct ctcttattgg caatatattc gataattatt aaatacataa atacaaaatc |
| | acattttgga atttaagaca ttttacaatt aaaaaaaaag tggaatcacg ttcaaaggtc |
| | gttgatagtc acaacttaac aatgacgcat taaagtattc aaaagtctat ttaactgatc |
| | tatgattgac acatagaaat gaagctatat aaaagttgta ctctcttttt gaaccatctc |
| | acaatcaaac tcaagtcaac ATGTATCAAA AATTTCAGAT CTCCGGCAAA ATTGTTAAGA |
| | CTTTGGGGCT AAAGATGAAA GTTCTGATAG CAGTCTCCTT TGGTTCCTTA CTATTTATAC |
| | TATCATACTC AAACAACTTT AACAACAAAC TTCTTGATGC TACAACCAAA Ggtaagaaaa |
| | ttatccatat cttgtgtttt attgttaagt caatgaatcc tcattttggt tttatgtttt |
| | cattttgttg tagTAGACAT AAAGGAAACC GAAAAACCGG TGGATAAACT TATAGGAGGG |
| | CTTTTAACTG CGGATTTTGA TGAAGGTTCT TGCTTGAGTA GGTATCATAA ATATTTCTTG |
| | TACCGCAAGC CATCCCCGTA CAAGCCTTCT GAATATCTAG TCTCTAAGCT CAGAAGCTAT |
| | GAGATGCTTC ACAAACGTTG TGGTCCAGAT ACAGAATATT ACAAAGAAGC AATAGAGAAA |
| | CTTAGTCGTG ATGATGCAAG CGAATCAAAT GGTGAATGCA GATACATTGT ATGGGTGGCA |
| | GGTTACGGGC TTGGAAACAG ATTACTTACT CTTGCTTCTG TTTTCCTCTA CGCTCTCTTG |
| | ACCGAGAGAA TCATTCTTGT CGACAACCGC AAGGATGTTA GTGATCTCTT ATGCGAGCCA |
| | TTTCCAGGTA CTTCATGGTT GCTTCCGCTT GACTTTCCAA TGCTGAATTA TACTTATGCT |
| | TGGGGCTACA ATAAGGAATA TCCTCGTTGT TACGGAACAA TGTCTGAAAA ACATTCCATC |
| | AACTCGACTT CAATCCCGCC GCATCTATAC ATGCATAACC TTCATGATTC AAGGGATAGT |
| | GATAAGCTGT TTGTATGCCA AAAGGATCAA AGTTTGATTG ACAAAGTCCC ATGGTTGATT |
| | GTTCAAGCCA ATGTTTACTT TGTTCCATCG TTATGGTTTA ATCCAACTTT CCAAACCGAA |
| | CTAGTTAAGC TGTTCCCGCA GAAAGAAACC GTCTTTCACC ACTTGGCTCG GTATCTTTTT |
| | CACCCTACAA ATGAAGTTTG GGATATGGTC ACTGACTACT ACCACGCTCA TTTGTCGAAA |
| | GCCGACGAGA GACTCGGGAT TCAAATAAGG GTTTTCGGCA AACCTGATGG ACGTTTCAAA |
| | CATGTCATTG ACCAGGTCAT ATCATGTACA CAAAGAGAGA AACTGTTACC TGAATTTGCT |
| | ACACCAGAGG AATCAAAAGT CAATATATCA AAAACCCCGA AACTCAAATC TGTTCTTGTC |
| | GCATCTCTCT ATCCAGAGTT CTCTGGCAAC TTAACTAACA TGTTTTCAAA GCGACCAAGT |
| | TCAACAGGAG AAATTGTTGA AGTTTATCAA CCAAGTGGAG AGAGAGTTCA GCAAACAGAC |
| | AAGAAAAGTC ACGACCAAAA GGCGCTTGCT GAGATGTATC TTTTGAGCTT AACCGATAAC |
| | ATTGTCACGA GCGCAAGGTC TACATTTGGA TATGTTTCAT ATAGTCTTGG AGGATTAAAG |
| | CCATGGTTAC TTTATCAGCC AACAAATTTC ACCACTCCTA ATCCGCCATG TGTTCGATCT |
| | AAGTCGATGG AGCCATGTTA CCTAACTCCT CCGTCTCATG GATGTGAAGC TGACTGGGGA |
| | ACTAACTCGG GGAAGATTCT TCCTTTTGTT AGGCATTGTG AGGATCTTAT ATATGGGGGG |
| | CTTAAGCTAT ATGATGAATT TTAGttctat tttatcacat ttgattttat tggattattg |
| | agtttttata atctaaggaa aaaatgctat ccgatccctc tttacagttt acacttgtgt |
| | cctcttctta tgtattaata tgttagtttt cttaaaacgt ttactaggtt tgtatggttt |
| | ataatattaa ataaaatgaa atttacatat atacttgtat cacttaaaat cattaagact |
| | ctaatttaat ttatatcatt gtgatgtttt ctcgaggtta ctttatgtgt catgaagata |
| | atggagtatt ggagttgtga ggtatcatgc gtcgtcgttg ttctactcta gtccaccttt |
| | aaagaatata aaaagagata tttaatcaat gttatgcgtt acaacatttt attatcgaaa |
| | aaacgttttg agtataaaag aaaaaataga gaaattttag tgatttccga gatataatat |
| | tcacctgcaa aagagagtgc tgattttaca caaatattga gagc |
| 6 | atcttccaat ataaagtctg aagcgcgggg tagtggagat ttgaacaatg gagtacataa |
| | aatagttcgg accccacctg tctttgatgg gaccatgcgc gcaaagcgct ctttcctctt |
| | ggatgatgcg tctgatggta atgaatctgg aacggaagag gatcaatctg cttttatgaa |
| | agaattggat agttttttta gagagcgaaa catggatttc aaacctccaa aattttacgg |
| | ggagggcatg aactgcctca agtaagcttg atacccatca ttatttggtc actttactgt |
| | gttacatttt aaaattttca gcaggagctg atatctaatc aatttctttg gcacaaggtt |
| | gtggagagct gtaactagat tgggcggata tgacaaggta cgggtcactg tgaatacgcc |
| | tgttgaatgt cacagcatct tttttgacaa gcaaatgtga cttcggcttt tcatcttttg |
| | ttccatcctg gcttacttgc ATGCGTACTG TTGTTCATGA TCTAGCAGTG GTGCTTTTGG |
| | TGATTTTCTA TGATTATTAT ATGCTTTTTA TACTGGATAG GTTACTGGAA GCAAATTATG |
| | GCGGCAAGTG GGAGAgtctt tcaggccccc aaagtaagaa gaatgctttt cttattagtg |
| | gtttgtctta gAAATTTTGG GAAATCATGT GGATATTTTT AAGAATTACC CTCTAATTGG |
| | TCAATTGTTT GTTCAGGACA TGTACAACAG TATCATGGAC TTTCCGAGgt ttctacgaaa |
| | aggtgagact atattcacca ccttttcctc tctctgcttt tggttcgtct atgtgacttt |
| | tgtatacact ggcatgggac tgggactcta tgtatcaacc cttctgagaa ataattgaaa |
| | tgattgaaca gtgaacaact gtgaatcatc ttgagatatg ttttccttaa gatacagtaa |
| | catcttgtaa cattatagTT TCTTCATTTT TCAGGCTCTT CTTGAATATG AGCGGCATAA |
| | AGTTAGTGAA GGTGAACTTC AGATACCCCT TCCGTTGGAA CTAGAACCGA TGAATATTGA |
| | TAATCAGgta aaattgagaa aaccatatca tgtgtctgta gtttttgttt gatcttcttc |
| | ttctgattaa tgtcagtgtt ttaacttaac ccactgcctt gtttctacac tagGCGTCTG |
| | GATCAGGGAG AGCAAGGAGA GATGCAGCAT CACGTGCTAT GCAAGGTTGG CATTCACAGC |
| | GTCTTAATGG TAACGGTGAA GTTAGTGACC CTGCAATCAA Ggtccggtag aatcttttta |
| | tatgtttcat tttacattca cactagatct ctcgtttttt ttttgtcaaa catttaatct |
| | atatctcata gtctgaacga acatactgtt ttgtaattaa tagGATAAGA ACTTAGTTCT |
| | TCATCAAAAG CGCGAAAAAC AGATTGGAAC CACCCCTGgt atgagttctg tttgatgaag |
| | aagtgttgtt ctcattttta ttttgaaact ttgacatggg ttatcactta catctcacaa |
| | tgtcatcagG TTTGCTCAAA CGTAAGAGGG CTGCTGAACA TGGTGCAAAA AATGCCATCC |
| | ATGTATCTAA ATCTATgtac gatttttggc tttgtggtct ggttttcaat gcgtgataat |
| | tcacatttga attctgattc cagttgttgt ttttcctagG TTGGATGTGA CTGTTGTTGA |
| | TGTTGGACCA CCAGCTGACT GGGTGAAGAT TAACGTACAG AGAACGgtaa aatcaattgc |
| | cactttctta aaaacctgag caatcacttt ctggttttac atatattaat aaactcttcc |
| | actatctgca gCAAGATTGC TTTGAGGTGT ATGCATTAGT CCCAGGATTA GTCCGTGAAG |
| | AGgtaagctc tcaaatctcg ttgtgtttac atatggatcc taagattgag tttagcactc |
| | agtttttgtc ttggcaacaa taatacagGT CCGAGTCCAA TCAGATCCGG CTGGGCGGTT |
| | AGTAATAAGT GGCGAACCCG AGAACCCTAT GAATCCTTGG GGAGCTACTC CTTTCAAAAA |
| | Ggtaaatgct ggttacatga tttttcagct tacacgtaga atgttgaatg acattttcaa |
| | acctccattg aaactgcagG TGGTAAGTTT ACCAACGAGA ATCGATCCGC ATCACACATC |
| | GGCTGTGGTA ACCCTAAACG GGCAGTTATT TGTTCGTGTG CCTCTGGAGC AATTGGAGTA |
| | Gaaacattta cagtttaaca aagcctttga agatctgaaa gagagaagat tgttagaagt |
| | agttgttgag agtattttgt ttgtatatta tgagagatta agcacaacat gagaagagcc |
| | tttaggaatc cttaattagg ccatctagtt tttattgtct ctcctctctt tgattagatt |
| | cttcttctaa gtgtcatcac tattgatttg ttgtagcacc aaacttcttt aaacctttct |
| | attaagaaca cacaaatcta caaccttttt atttttttta attgtttatg tgatttgttt |
| | tctgtggcag tgaatttttt atattatcaa cttatcatgt tagctcaaga ttgcatctca |
| | atttgtactt atcttagtgg taattagaaa aaaaaacaaa attaggctac aatagttttg |
| | tttgtttgtt tgtttaggtg ttagggatag ggtttatttt ttccgaagtt tattagtgtt |
| | tactatttag agtttaatgt t |
| 7 | gaaagtatta tgataaagaa ggattaaaaa aaaaaaaatc ttcttaatat agcttacaat |
| | gttttgttgt taaagtatag ctaagtaaag tatgttataa atggtgcatg attttttatt |
| | tttgattaaa aagtggtaaa tgatattttt ttcctccatt ttgcattttt acactttgta |
| | tgatccaatt tgcttttatt tatctacata taataaatct ctataataaa ccatttacat |
| | accattacta aaactaaaat tataatggaa aaatattatt atgttattta ttgttacttt |
| | ggtaaagcat tattatttat tttgcttatt ttaagggcta ataattaatt gaaattaagc |
| | agttgacgaa agtttttttt attaatttat aaagcacaac atttccttgt ctacacgatc |
| | ataaagctca caaagagaga attgagaaga aacaaactcg tcggagaatt cagtactcgc |
| | cgaagaggaa gaagaagaag ATGTCTTGGC AATCATACGT CGATGATCAC CTTATGTGTG |
| | ATGTCGAAGG CAACCATCTC ACCGCCGCCG CAATTCTCGG CCAAGACGGC AGTGTCTGGG |
| | CTCAGAGCGC CAAATTTCCT CAGgtttttt tacttcttca tcctctcttt tcgccttact |
| | acgatccgtc gcttgaattg tcggaatcct ccgtgatcgg atctgacgaa tctcggatct |
| | gattttgaat ttttcaatct ccggaatctg atgaatattt tcgatttgca tttctaaatc |
| | tatcgatccg tatgcgaaat tgaattcaaa cgtagggctc tagaccatta gtctattgtg |
| | agatttcttc ggtatcagaa gttattagat cgtagcttcc atagaagaag atccatatgc |
| | ttgtgaaatt gtacgcatgc gtgtgcaacc atcgatgcaa ggtcttcttc ttcttgtagg |
| | catgtagatt ctatggtctt agtcagaatt actgcttaac aattgcatct tggataatct |
| | ctgtttccat ttttcttata tgcttgagga aatgttttga tcaatagcct aaaatgttga |
| | tttgattttg ccaaaatctg atgatgtgtt attgataatg tgtgtttagT TGAAGCCTCA |
| | AGAAATCGAT GGAATCAAGA AGGACTTTGA GGAGCCCGGG TTTCTTGCCC CAACCGGACT |
| | ATTTCTCGGT GGCGAAAAAT ACATGGTTAT CCAAGGTGAA CAAGGAGCTG TGATCCGAGG |
| | GAAGAAGgta actttcttta cttcatacat cagaaagctg catgtagatt ttgatagaga |
| | atagaatcgg aattcatgta acaatctgtg aatcttcagG GACCTGGAGG TGTCACTATC |
| | AAGAAGACAA ACCAAGCTTT GGTCTTTGGC TTCTACGATG AACCAATGAC TGGAGGTCAA |
| | TGCAACTTGG TTGTCGAAAG GCTCGGGGAT TACCTTATCG AGTCTGAACT CTAAaaccaa |
| | ggtttcattt caggttcttc ttaactaaag agtgtcaatg cactttttat tgtgattgat |
| | tgtaatgctt tcaaacacaa atcatttgtt actttagaac caattgtgat tgattggtct |
| | ccttcgttac cgagtttgag tttgtgtgtt cttgtaatga catttgatca tcttttttct |
| | ccatatgtat tgagttttga tttttgtttc ttcatattat tactttttct tgaaatgatc |
| | tgctgtttat gatttggggt tcaaaatatt tttggtttgg caaacaagga agagtttgcc |
| | aagtattagt agcaagtgct atgagtattt tcggcttggc gaacatcttc gtgtacacgt |
| | gtgacataac aaacctattt gagaatggtg taagctaggt agatattaca taaacgatgt |
| | aagttgggaa ttcgtttagg agagagatat tgtatggtaa gaatttcact tcgaattctc |
| | tgcttcaacg tggc |
| 8 | agaagactag gcggaacatc tcatcaaaac cctatacatt caacagggaa attcttttgc |
| | acgaatgtta gacttcaata ttgaataaaa ttcatagttt caacaatctc ataaaaaaag |
| | agctgggctc cattcgaaga cacattaatt tccatgggcc tggtccacat acaaccatac |
| | taaatttgaa gtaatttacc cgccatttaa aaaagcccat aggctccttc tcctagaagc |
| | tggcgggaaa atcccaaaac ttttcccggg aaagtagata aaaaatttcg gccattaaag |
| | gacaaaatca caagaaagta gaaaccctag agattttgaa accgaaaccc caaaaacccc |
| | tttgacgcct ccttgttctt atctctttat aaaaaaccat ttctttcctg caacatcgtt |
| | gcttatcatc agacgcacat cacctgttcg ataaaattcc tctgagagtg ttttttttgt |
| | tttccttctg acaaagaaat ATGTATGTAG TGAAGCGTGA CGGAAGACAG GAAACTGTTC |
| | ATTTCGATAA GATTACTGCG AGGCTTAAGA AACTTAGCTA TGGGCTTAGC AGTGACCATT |
| | GTGACCCTGT CCTCGTTGCT CAGAAGGTCT GTGCCGGTGT CTATAAAGGA GTCACTACGA |
| | GTCAACTTGA TGAGTTGGCT GCTGAAACTG CTGCTGCTAT GACTTGTAAC CATCCTGATT |
| | ATGCATCTgt gagtatctct cttcgttttc ctttctgggt attgcttgat tttgattagt |
| | cgtttctgga gaagtgatct ctgtcattgg attggtgttt catttgattg aattgatctg |
| | tataatttac atgttatctg tgttcatatg tcagCTTGCT GCTAGGATTG CTGTGTCGAA |
| | TCTCCACAAG AACACTAAGA AGTCATTTTC TGAGACgtga gtgttgagtt ctttcttagt |
| | gtgtattata cccttgatat gagttcaagt ttccatgtgt gttgactccg atggcttgtg |
| | tggtatcttg cagGATTAAG GATATGTTCT ATCATGTCAA TGATAGATCT GGACTAAAGT |
| | CCCCACTAAT AGCCGATGAT GTGTTTGAGA TAATTATGCA Ggtaaagaaa tcttgtgtta |
| | agctcttgat tcaatctgtt tcttggtgtg atatatatat atatatatat gtatgtatct |
| | tataaatcac tgacttgtgt gttactggtt tcttcagAAC GCTGCTCGTT TGGACAGTGA |
| | GATCATCTAT GACCGTGATT TTGAATATGA TTACTTTGGA TTTAAAACTC TTGAGAGATC |
| | GTACCTCTTG AAAGTCCAAG GGACTGTTGT TGAAAGGCCT CAACACATGC TGATGAGGGT |
| | TGCTGTTGGG ATCCACAAGG ATGATATTGA TTCCGTGATC CAAACCTACC ATTTGATGTC |
| | TCAGAGATGG TTCACTCATG CATCTCCTAC TCTCTTCAAC GCAGGAACTC CAAGGCCTCA |
| | Agtaaatacc tatcacttga tatttattat atctattaaa taaggcgttt tactttgata |
| | cgtgtctttg ctgatctgct attgaaaata attgaaattg cagTTAAGTA GCTGCTTTCT |
| | AGTCTGCATG AAAGATGATA GCATTGAGGG CATATATGAA ACACTCAAAG AGTGTGCTGT |
| | TATAAGCAAA TCTGCTGGGG GTATTGGTGT TTCAGTTCAT AATATTCGTG CTACCGGAAG |
| | TTACATTCGT GGCACAAATG GAACATCTAA TGGTATTGTT CCTATGCTGC GTGTATTCAA |
| | CGATACAGCT CGTTATGTTG ACCAAGGAGG AGGCAAGAGA AAGGgtacgt atcagctctt |
| | tgtactatta gcataatcat ctgtccagta tatggtctaa agtgtatctg atttataatt |
| | tgtaattggt gaagGAGCCT TTGCTGTTTA CCTGGAGCCA TGGCATGCTG ATGTCTATGA |
| | GTTTCTGGAG CTGCGAAAGA ACCATGGAAA Ggtatagtca tagctagata attcaccata |
| | tctactccct aaatgtgatt accatttgac gctgatacaa cctcttaata cactttgtcg |
| | cattgcagGA AGAACACAGG GCTAGAGATT TGTTTTATGC TCTCTGGCTT CCAGATCTTT |
| | TCATGGAGAG GGTCCAGAAT AATGGGCAGT GGTCACTGTT TTGTCCTAAC GAAGCTCCAG |
| | GTTTGGCAGA TTGCTGGGGA GCTGAATTTG AGACACTGTA CACTAAGTAT GAAAGAGAGg |
| | tgagtcccta tttcatccat gtatatgctg cttctttagt aactcaaatt cctgttatct |
| | caatacagtt atgtttgttc atatcttcag GGAAAGGCCA AAAAGGTTGT TCAGGCGCAG |
| | CAGCTTTGGT ACGAAATATT GACATCCCAG GTAGAAACAG GAACACCATA CATGCTTTTC |
| | AAGgtaagta acagtcatca ttctgtagct acacgttatg gccttataat cattggttct |
| | tactccaaat ttgaatgctc ttaaactata gGATTCATGC AACCGAAAAA GTAATCAGCA |
| | AAATCTGGGT ACCATAAAGT CGTCCAACTT ATGCACTGAA ATCATTGAGT ACACTAGTCC |
| | AACAGAAACT GCTGTGTGCA ATCTTGCATC TATTGCTTTA CCCAGATTTG TAAGGGAGAA |
| | Ggtgagaggg agactggttt tttaaaattt gctttctctt tattactcaa tgtatagctc |
| | taacattctt catctcacaa cagGGTGTCC CATTAGACTC TCATCCACCT AAGCTCGCTG |
| | GCAGTCTGGA CTCAAAGAAT CGTTACTTTG ATTTTGAAAA ATTAGCAGAG gtcagataca |
| | agcactcgcc ttgcttgacc tgaaatctga ttcttaagga attatctgtg gagatatttc |
| | cgtgtctgtg atgtgatgtt tgacttttta atttttctgt gtggccagGT GACTGCTACT |
| | GTTACTGTTA ATCTCAATAA GATAATAGAT GTGAATTACT ATCCTGTGGA GACTGCAAAA |
| | ACTTCAAACA TGCGTCATAG ACCTATTGGT ATTGGTGTAC AAGGCCTTGC AGATGCATTT |
| | ATCCTCCTTG GAATGCCATT TGATTCTCCA GAGgtagact tgttttgaat tatgatcaat |
| | cttggaaaat ataattttgt tatctgttct taagcagttt aatttgttac tcagGCCCAA |
| | CAACTGAATA AGGATATATT CGAAACCATA TACTACCATG CACTCAAAGC ATCTACAGAG |
| | CTTGCTGCAA GACTTGGCCC CTATGAAACC TATGCTGGAA GTCCCGTGAG TAAGgtatgc |
| | atctcagcca tcaattatat caatttggtt ttcccaaact tcataagcta ccattgtgga |
| | ttgttatgct gactttatcc catgcttctc tagGGAATCC TTCAACCTGA CATGTGGAAT |
| | GTAATTCCAT CAGACCGCTG GGACTGGGCT GTTCTTAGAG ATATGATATC AAAGAATGGA |
| | GTGAGGAACT CTCTTTTAGT AGCACCAATG CCAACTGCTT CAACCAGTCA AATCCTTGGG |
| | AACAATGAAT GTTTTGAGCC CTACACATCA AACATCTACA GCCGCAGAGT CTTGAGgtat |
| | gtgaatatta aatcatttga caagtatgtt tctggttttc cccatttgat gcttactcac |
| | ttggttgtct tggtttgtac agTGGTGAAT TCGTAGTGGT TAATAAGCAT CTTCTCCATG |
| | ACCTAACTGA TATGGGACTT TGGACTCCAA CGCTGAAAAA CAAATTAATT AATGAGAATG |
| | GTTCTATAGT TAATGTTGCT GAGATACCTG ATGACTTGAA GGCGATTTAC AGgtatagct |
| | tccacttatt ttgtgttttc actctctact gtctagataa agaaatttga cttgtttctt |
| | ctgtaaaaca acacagAACT GTCTGGGAAA TCAAACAGAG AACAGTGGTG GACATGGCTG |
| | CTGATCGTGG ATGCTACATA GATCAAAGCC AAAGCTTAAA CATACACATG GACAAACCCA |
| | ACTTCGCAAA ACTCACTTCG CTACACTTCT ATACTTGGAA AAAGgtacaa accttaatca |
| | tctaaactct tcatatgata attgtgaaat aggttagaga ttctatagag tatctgatcc |
| | ttcactcatc tgacaattac tcttaatctc acttatgttg ttgtgaatct accttaagGG |
| | TCTGAAAACC GGGATGTACT ACCTGCGATC CCGTGCTGCA GCTGATGCGA TAAAGTTCAC |
| | CGTTGACACA GCCATGCTCA AGgtagaaaa aacaatgcaa actctttacg ctgattcttc |
| | ttgtgaactc agacatttta cctatgagtt gttttcgttg gggtgaatgt agGAGAAGCC |
| | GAGTGTAGCA GAAGGAGACA AAGAAGTAGA AGAAGAGGAT AATGAAACTA AGTTGGCGCA |
| | GATGGTATGT TCCTTGACAA ACCCTGAAGA GTGTTTGGCC TGCGGAAGTT GAagctctaa |
| | gttatagttt gggtcttaaa aagttagaaa gtaaaagcat gtctcttgga cggtcttttt |
| | tatttacttg cttatctggg tgtattttgt taatagtttc ctaatgctta atgttgcttg |
| | agtttttgtg taatccaatt tcgtttttac cttttctctt gaaacaataa ggatttgtaa |
| | cgagaattat gtataaccac caccacctta cggtagattt tactatccat atataaatat |
| | tttaccatcc atttataaat atttgtagtt tggtactact accaatggtt gtaagtaatc |
| | tgtaagaata tattctgatc attgtagatt agaaaatgtg ttactacagg tttcactagc |
| | ttatcctaga actagaaaca tgaaaattat gtatcgaatg gtgaaaatat taatacaaac |
| | atatttacgt ttaaatgcat gtgtacacaa caaagtttct aaagcaagct ctatcatata |
| | gagaataaag ta |
| 9 | ttgctttagg tatccatata gttttgaccg acctcgatga tcatgttata ttctgtggag |
| | atttatcaac tatttataaa taccttgaaa ccgctactag acattggagt aatccctcac |
| | cttgtctcat ttggcaaata tttcctatag gttcaactta ttagtagaaa tgacaatgtc |
| | ttggctgaca cttatcaaga actctccttg taatcactta gttacttcca ttatggaaaa |
| | gttgaccgat cgaaaaaagg tattaaaaaa aaaaaataga aaaattaaga ttttcatagt |
| | gtaattgtaa aaaataaaat caaattattt tcagatattc cgtattggga ataaatctca |
| | gccgttgatt actatcaacg gtgtacaatt actgcctttg cctgttactt gttctgctcc |
| | gtcgctcaga taggatctca acaagacacc acaaacccta aatttcgtca actccacagc |
| | gactcgattc gatcaaggaa ATGGCGTACG CTTCTCGTTT TCTCTCCAGA TCTAAGCAGg |
| | tatatactct ctctccctcg atttttctga ttctcttctt cgttctgttt gattcctttt |
| | gttttcctcc catttctggg ttttatgtgt ttcgatgcga tggttagagt gagattatcg |
| | attttactgt atctctatca ctgaatcaca tcttagggtg tgccatttca atatcgtagt |
| | cgaatttttg ttatctttcg tacgatctca atcggagagt ttgttgaaat caaatgataa |
| | atttgatggg gtttttttct actcgttgtt gatttctaat acagttcgaa atgataagat |
| | gatttgcaag aagtattctt ttcatcaaaa cttgttattg atccataatt tttattatct |
| | tactctcatt acgcagCTAC AGGGGGGTCT GGTCATTTTG CAGCAGCAAC ATGCTATTCC |
| | AGTCCGAGCT TTTGCTAAGG AAGCTGCTCG TCCAACCTTT AAAGGAGATG gttagtgacc |
| | aaaactcata cttcggattt gttattatgc atagaacatt acgttttcaa taacacacct |
| | agttgaaaac agttgctttc ctttctttag cccttcgtgc ttttgagttt aacatcgtga |
| | ctacttaaga atatgtcaag tcactttttt tatgtcgaat gtgtagaaaa actatattgg |
| | tcaatgtaat ataatcttgt gaaacccagg ccatgattgc taggactgtt gttctgctta |
| | cttcttttgt tgagttttat atgtatccag tttatgatgg attatgttta atatgttgct |
| | gaaatctgta ctatgtgttt agagtgaaga agcattgctg tttactatta ttgactcaag |
| | ttttacactt tttgacagAG ATGTTGAAGG GTGTCTTTTT TGATATCAAG AACAAATTCC |
| | AGGCTGCTGT TGATATTCTC CGTAAGGAAA AGATCACCCT TGATCCAGAG GACCCAGCTG |
| | CCGTAAAACA GTATGCAAAT GTAATGAAGA CCATCAGGCA AAAgtaggcc tcttgttact |
| | cttttgtagg tgtttgttat ttagcttgaa tcttgtatgt cgtgatctct atttctgttt |
| | gttgggattg gttttacttt tcgacttttc tgaaacgagt taaatatatg tgtcaatgct |
| | gctattttaa ccttgttaat ttggttgctt gtcatccgtt tttttggtat gcagGGCAGA |
| | CATGTTCTCA GAATCTCAGC GCATTAAACA TGACATTGAT ACTGAGACTC AAGACATTCC |
| | AGATGCTCGT GCATACTTGT TGAAGTTGCA GGAAATTCGC ACCAGgtagc tgttagactt |
| | tgaataattt tcagttatct taggatagtt ttccctcacc cgtaaacttg ctcttcttat |
| | gttattataa tattggaatt atcttcctgt aagatcttga atgtgatcgt taagcagtta |
| | tctgaagact gcatttaact atctatattt tcatctccct ctttgatctg ctattgtttg |
| | caacatatga agaattgttg gaagcagtct ttagttatac tcccacttgt gatatatctt |
| | gcagGAGGGG GCTTACTGAT GAGCTTGGTG CTGAGGCCAT GATGTTCGAG GCTTTGGAGA |
| | AAGTCGAGAA GGACATAAAG AAGCCTCTCC TGAGAAGTGA CAAGAAAGGA ATGGATCTTT |
| | TGGTTGCAGA GTTTGAGAAA GGCAACAAAA Agtgcgtcat cattcttcaa ccatccatac |
| | aaaacacgaa caaatgattc tcattactac ttatatgtat atcgatttac atattgatag |
| | ctaattgaat tgcatgtttg cgtctcatta atctaaacag GCTTGGGATT AGGAAAGAAG |
| | ATCTTCCTAA GTACGAAGAA AATTTGGAGC TCAGCATGGC CAAAGCACAG TTGGATGAGC |
| | TGAAGAGTGA TGCTGTTGAA GCTATGGAAT CTCAGAAAAA GAAgtgagtt ttgttttctt |
| | ttcacttttt ttgtttctca atttatcaat cattgatctt actcatgtca taacgcgatg |
| | gaacttgcgg attattcagG GAGGAATTCC AGGATGAGGA AATGCCGGAC GTGAAGTCTC |
| | TAGACATCCG TAACTTCATC TAAggtttga tccttagaaa catttgattt gttgtaagaa |
| | aaggcaaaga tctctcactt gattgtcttt gaaagagaag atcgttccct tgctgctgtt |
| | ttggtttggc gttcaataag gtctctcacc tggatttgag tctaactctc tctgtggtta |
| | ttacgcttga gattcttaga cacaaacgtt gtttcatgtt tttttgataa tggtgatcac |
| | tggaatttga gataattaat aaaagttgtg atgttaattc gaaacaaaag cgtggcaagc |
| | aaaatcaacc cgagaaacta ttatagtttt gtatttagta gaccaaattc gaaccaaatc |
| | taaccgaaat gggatctgga gtatcataca ttctagatga attaaaccaa tcatatcgaa |
| | cacgtggctt gtctgtgaac aattataatg ggtttgtctg agagacgtta acaactgttt |
| | tcttcgccat ggcggcgatt cctctcaaag ctccttctct tcc |
| 10 | tttcgatcag ctttttcgat tttggatcta ttttctatga aatatcagat ctggtgattg |
| | ttttacatat ttttgggttg aattcacaag attttctgga aacgagatcg attaattgag |
| | ttttctgtgt ttttatctta agctagatct cgatttctat gtttttggat tgatttgata |
| | agattttcga gaattttttg tgtttttgtc aaagttcgat ctcgatttct atatttttgg |
| | ttgaattcac aagactttct ggaaacgaga tcgattttgt gagttttctt tgtttttaat |
| | ctcgattttt ggattgattt gagaagattt tctgaaagcg agatcgatgt ttttggggat |
| | tttctttgtt ttgttcaata attcggtctc tgttttctta tcaaaaaatt cgttttccat |
| | ctcaaatcga tgttcttatt gatttaattg agttttagtt tgcagggatt tgatcgttgg |
| | taagctatct ttcagcaaac ATGCATGGTT ATGAAGATgt aagcacgctc atgaattttt |
| | gttttcagtg attttgtcga attcaattta aggtagatag atttgacatt gttcgataat |
| | gttatattgc agGACCTTGA TGAGGAAGCT GGGTATGATG ACTATTACAG CGGTGATGAG |
| | GATGAGTATG AAGATGAGGA AGAGGAGGAT GAAGAACCTC CTAAGGAAGA ATTGGAATTT |
| | CTTGAGTCAC GCCAAAAGTT GAAGGAATCA ATTCGGAAGA AAATGGGAAA TCGAAGTGCT |
| | AATGCTCAAT CTTCACAAGA GAGAAGAAGA AAACTTCCTT ATAACGAgta tgtggtggct |
| | aaatcacatt ttctaattca ttacaatgtc ctggaatgtg ttttgatgct gagcttattg |
| | atttttctta atgcagCTTT GGTTCTTTCT TTGGTCCTTC ACGGCCTGTT ATTTCCTCAA |
| | GGGTTATACA AGAAAGCAAA TCCTTGCTTG AAAACGAGCT ACGTAAAATG TCGAATTCGA |
| | GCCAAACTgt atgtgcattt gatctttgtt actctttgta tttttatcat ttaagATGTT |
| | TTTGCTGATG GAATTGTTTT TTGGGGTGCA GAAGAAAAGA CCAGTTCCGA CGAATGGTTC |
| | AGGCTCTAAG AATGTGTCAC AAGAGAAGCG ACCTAAAGTT GTGAATGAGG TGAGAAGGAA |
| | AGTTGAGACT CTTAAGGATA CAAGAGACTA TTCGTTTTTG TTTTCCGATG ACGCGGAGCT |
| | TCCTGTTCCG AAGAAGGAAT CTCTTTCACG AAGTGGCTCT TTTCCTAATT CTGgtatgtt |
| | gtgtcttttg aaaaatcttt ttcgctattt gtgatcttta agCATACCAT TTTCATGAAG |
| | ATAACTTATA CAGGTTTTTT GCTGATGTTC AAGAGGCTCG ATCTGCTCAA TTATCATCGA |
| | GGCCCAAACA ATCATCAGGT ATCAATGGTA GAACTGCTCA CAGTCCCCAT CGTGAGGAGA |
| | AGAGACCTGT TTCAGCGAAT GGACATTCAA GACCGTCTTC CTCGGGCAGT CAAATGAATC |
| | ATTCAAGACC GTCTTCCTCT GGCAGTAAAA TGAATCATTC AAGACCGGCT ACCTCGGGCA |
| | GCCAAATGCC AAATTCAAGA CCAGCTTCCT CTGGCAGCCA AATGCAGTCG AGAGCTGTCT |
| | CAGGCTCAGG GCGACCTGCT TCCTCAGGCA GCCAGATGCA AAATTCAAGA CCACAAAATT |
| | CAAGACCAGC TTCCGCTGGT AGCCAAATGC AGCAAAGGCC TGCGTCCTCA CGCAGCCAAA |
| | GGCCTGCGTC CTCAGGCAGC CAAAGGCCTG CGTCCTCAGG CAGCCAAAGG CCAGGTTCGT |
| | CGACAAACCG TCAAGCACCT ATGAGGCCAC CAGGTTCAGG TTCCACAATG AATGGTCAAT |
| | CAGCCAACCG GAATGGCCAA CTGAATTCCA GATCAGATTC CCGAAGATCA GCTCCTGCTA |
| | AAGTGCCAGT GGATCATAGG AAACAGATGA GCAGTAGCAA TGGAGTTGGT CCTGGTCGGT |
| | CAGCGACCAA TGCAAGACCT TTACCTTCTA AGAGTTCATT GGAAAGAAAA CCCTCAATCT |
| | CGGCGGGAAA GAGTTCTCTT CAAAGCCCTC AGAGACCGTC CTCATCAAGA CCAATGTCAT |
| | CTGATCCTAG GCAACGGGTA GTAGAACAGA GAAAGGTTTC TCGTGACATG GCCACACCCC |
| | GAATGATACC TAAACAATCA GCGCCTACCT CGAAACACCA Ggtatcatga tcatgatctt |
| | tcacatctct ttcttttgtc cttcctctag ccaaggcact aatttgtcaa gtaatattta |
| | cagATGATGA GTAAACCAGC GCTCAAGAGA CCTCCCTCGC GTGACATAGA TCATGAAAGG |
| | AGGCTGTTGA AGAAGAAGAA GCCTGCAAGG TCAGAGGATC AAGAAGCATT CGATATGCTT |
| | AGACAGTTAT Tgtaagtatt gctccaaact ttcttcctac tctcaaattg taagttacaa |
| | ttttctaatt ctattttgtc tcctgatact taaatggggg tttgtgtatc aattttagAC |
| | CACCCAAGCG GTTTTCTCGG TATGACGATG ATGACATAAA CATGGAAGCA GGCTTTGAAG |
| | ATATCCAAAA GGAAGAGAGA CGAAGgtaca tgagtatttt tgttatcaca cgtttcattt |
| | atttgtgttt cttggatatt ccttaacgat tgaattggtt gttaaatgca gTGCGAGAAT |
| | CGCAAGGGAG GAAGATGAAA GAGAACTTAA GCTCTTAGAG GAAGAAGAAA GGAGAGAAAG |
| | ACTGAAAAAG AATCGGAAGC TGAGCCGTTA Gaagaatcct ttctcctttg tgtctttgtc |
| | ttcttttagg acttttttag tgttttctca ttgaaatctc tttggccgct tgaggcaaaa |
| | aagagtttga cctttttttt gttttgtgtt ttcaaattaa ggatcttttt tttgttcatg |
| | gaaattgtac aattagaaat aatatctttt attggggaca cttcaagaag aatctgttgg |
| | aaaccttccc agttagtgaa agcttgattc tctttttttt ttttggagta aagctaaaac |
| | cagaggagga tgataaagaa aaagaaacaa agaatatttc tttattcacg tgtagagttc |
| | ctttagctga taaaatttca ctttttatga gtctgataac atgattttag tgattctttg |
| | tctcttttat tctttggcta aacaaattcg ttgagaaatc aaatggtgac caaagaagaa |
| | gattgccttc ctcctgtaac ggagaccacg tcgagatgtt attctacttc t |
| 11 | ataccggaaa tgtcgtaccg tcctgaacat aatgcacata atttgactgt agctaggctg |
| | taaaagattt taacaaaatt gttttagaat aaaattataa gtttaaaagg tatggtttga |
| | cttgaactgt actggaattt ataccggaaa tatcgtaccg ttctgaacat aatgcacata |
| | atttgactgt agttaagcag taaaagattt taacaaaatt gttttaaaat aaaattataa |
| | gtttaaaagg tatggtttga cttgaactgt accggaattt ataccggaaa tgtcgtaccg |
| | tcttccacac ttcggagaaa cgacagataa gctctctctg ttctcttgcc acacttccca |
| | atacatggat ccattttgac gtcatcttta tcactatctc tctattatat aaatctcttc |
| | gtaccctttt accgattctt caccgtgatc gcttaatcag acctcaattt cgttgttaaa |
| | gaacaaagct ttaagcagcc ATGGATCCAA ACCAACGTAT CGCGAGAATC TCTGCTCATC |
| | TCAATCCTCC TAATCTTCAT AATCAGgttc aaatttcgtt gaattctctg attcttaaac |
| | caatttggtg atcgaagttt gattcttttt tttttgggtt gatctgattt cgatgatttg |
| | gatttagATT GCTGACGGGT CAGGTTTGAA TCGGGTGGCT TGTCGGGCAA AAGGTGGATC |
| | ACCCGGATTC AAAGTGGCGA TACTTGGAGC AGCTGGTGGA ATTGGACAAC CTCTTGCGAT |
| | GTTGATGAAG ATGAATCCTT TGGTTTCGGT TCTTCATCTC TATGATGTTG CTAATGCTCC |
| | TGGTGTTACT GCTGATATTA GTCATATGGA TACTAGTGCC GTTgtaagtt ctaaattctc |
| | cggttttcga ttccaaaatt actactttag atgttttaga gctaataaaa ttgatcaata |
| | gtgatgattg ttgttgttga aatagagaaa tgagcttaaa gatcatatac atgagcttaa |
| | aaactagtac tttagatgtt gtagagcact agtgatgatt gttgttgtta agatcatata |
| | gagattgttg tgaatgtttt tggaaaactt tgttttagGT TCGTGGATTT CTCGGGCAGC |
| | CGCAGTTAGA GGAAGCACTT ACGGGTATGG ATTTAGTGAT CATACCTGCT GGTGTTCCGA |
| | GGAAACCAGG GATGACGAGG GATGATCTGT TTAACATTAA TGCTGGGATT GTGAGGACAC |
| | TCTCTGAAGC TATAGCTAAA TGTTGTCCTA AAGCAATTGT GAATATAATC AGTAATCCGG |
| | TGAACTCCAC GGTGCCAATC GCAGCTGAGG TTTTCAAGAA AGCTGGAACC TTTGATCCAA |
| | AGAAACTCAT GGGTGTCACT ATGCTTGATG TTGTTAGAGC TAATACCTTT GTGgtatgca |
| | ctcattattt ggtcttagaa tggtgtttag tattgtccat tagaactcaa ctatcttctt |
| | ctttgcattt atggggttga atagGCGGAA GTAATGAGTC TTGATCCCCG TGAAGTTGAA |
| | GTTCCGGTTG TTGGAGGACA CGCAGGAGTT ACGATTTTAC CACTGCTTTC GCAGgtttga |
| | gatcagatga ttctcatcat tatgtttgtt tgaagcagat ataatattct catcattatg |
| | ttggctacag GTGAAACCTC CTTGCTCGTT CACTCAAAAA GAGATTGAAT ATCTCACAGA |
| | CCGCATCCAA AACGGTGGCA CTGAAGTTGT TGAGgtataa actaatcttt cagctttctt |
| | tgttttgaac ttcgaattaa gcggtgcatt taccgtttaa atcattttgc agGCTAAAGC |
| | TGGAGCAGGT TCTGCAACAC TATCCATGgt aggtcttttg ttgtaacatg ggagttgtat |
| | gacaaagctg ggaatttgat tgatatctca atctgttaaa tgataaaata cagGCATATG |
| | CAGCAGTGGA GTTTGCAGAT GCTTGCCTCA GGGGTCTACG AGGTGATGCA AACATCGTTG |
| | AGTGCGCATA TGTGGCATCC CATgtacagt cctttaattc aactgtacaa tattgtatct |
| | ataaaagatc tcttaaccct aaaagatgaa catatggact ttgtcttatt cctcatacag |
| | GTGACTGAGC TTCCCTTCTT CGCATCGAAG GTGCGTCTGG GACGATGTGG GATCGATGAA |
| | GTGTACGGCC TTGGACCATT GAACGAATAT GAGAGgtaaa agttaaaatc ttgatcgatc |
| | tgacatcttg aatttacttc gacatgtttg tatgttcata tcgtttttcc gccctttctt |
| | tttgctaatt gatcagGATG GGATTAGAGA AGGCAAAGAA AGAGCTTTCA GTAAGTATTC |
| | ATAAAGGTGT TACCTTTGCG AAGAAATAAa gagactcgat cgtgaataaa cacacttaag |
| | cgatggtttt ggaatagtca gagttttgga ataagaataa tgcctcacaa taaaagctct |
| | tgcggtcttc ttggatccaa tcttaaaggt tcaagaaact catctccttt aggtaaaatc |
| | ttcgattgtt ttatcgttcc atcgaaccac tttgttctta gatacaagaa cgtttatgat |
| | ttatgtagtt gggctataaa agtgagaaca gagcaataat cttgcaacat tttttctcat |
| | cttcttggtg tgtttttttt ttgttggttt tcatcttttt gttcttgctc atgagagcat |
| | ctttagaagg ctattgttgg gaagtaaata agtttgcatc gcggaaaaga tgatcaaggt |
| | cattcgggat acctcatacc tgtcatttga gttcatctaa gtaacttctt acgcttttag |
| | gctatctacg gttgttctta ggatttaggt gttagtggtt atgctatta |
| 12 | aacaaaaata ctcgaattca aacttaagca gtcacagtaa cttcgtgcag gagcttaccg |
| | gagatgaatt catcataaac cggcgacggt agcggcggag caaagcaaaa atgcgatgat |
| | tcatggaata ggtctcaaaa gtcacgagag gatcacgtga gatatcttga aaagaatcgg |
| | acggctaaga ataaagcaga ctaattctct tatctatctc taaccgttaa ataaaaacta |
| | aagttttaac cttttaacct gggactaggg ttttcagatt tcactactct tgtcgtgtaa |
| | gacttgagca actatataat ctcaactttt ctcaatcact atccgctgcg gtctcgccgt |
| | gctgcccaca acaatctccg acttcgtctt cctcatctat catcgtcgtc gtcaacctta |
| | tttatctctt aatttatcat taaaaccaaa aaaccaaaaa aaaagcctta gctttcgttt |
| | cttcaatccc agcaaaaaaa ATGGCTCAGG TTCAAGCTCC TTCTTCACAT TCTCCTCCTC |
| | CTCCTGCTGT TGTTAACGAC GGGGCTGCGA CGGCTTCTGC TACCCCTGGA ATCGGCGTCG |
| | GCGGCGGTGG AGACGGAGTC ACTCACGGTG CTCTTTGTTC TCTCTATGTC GGAGATCTGG |
| | ATTTCAATGT CACCGATTCT CAGCTTTATG ACTATTTCAC CGAGGTGTGT CAGGTTGTAT |
| | CTGTTCGTGT TTGTCGTGAT GCTGCTACCA ATACTTCTCT TGGTTATGGT TATGTCAACT |
| | ACAGCAACAC CGACGATGgt ttgtgcccta aaaatttccc cttttttttg ttgattgata |
| | acatttgata ttttggtaaa gatctgattt ttcggttttg gaatcattcc tttggctagt |
| | ttgattgatg ggttttgttt gattttgtta atagatatta atttacacga atttaaaatg |
| | ttgacactga ttagggattt tgttatcatt gttgtttttt gtaatgtcag CGGAGAAGGC |
| | AATGCAGAAG TTGAACTACA GTTATCTCAA TGGGAAGATG ATTCGGATTA CTTACTCTTC |
| | TCGTGACTCT TCTGCCCGTA CAAGTGGGGT TGGGAATTTG TTTGTAAAGg tatattcttt |
| | gtttgatgtc tcttatctag cagcttctct ttttgtttga ttgcctaatt atgtattctt |
| | tctttatgtg aagAATTTGG ATAAGTCAGT TGACAACAAA ACTCTGCACG AGGCGTTTTC |
| | CGGGTGTGGG ACTATTGTGT CCTGTAAGGT TGCTACTGAT CACATGGGTC AGTCTAGAGG |
| | ATATGGGTTT GTGCACTTTG ACACTGAGGA TTCAGCTAAG AATGCTATTG AGAAGCTGAA |
| | TGGGAAAGTG TTGAATGACA AACAGATTTT TGTTGGACCT TTTCTTCGTA AGGAGGAAAG |
| | AGAGTCTGCT GCTGATAAGA TGAAGTTTAC TAATGTTTAT GTGAAGAATC TTTCGGAGGC |
| | GACTACTGAC GATGAGTTGA AGACTACTTT TGGTCAGTAT GGTAGTATCT CGAGCGCTGT |
| | AGTTATGAGG GATGGAGATG GGAAATCCAG GTGTTTTGGA TTTGTCAACT TTGAGAATCC |
| | TGAAGATGCA GCTCGTGCTG TTGAAGCTCT CAATGGAAAG AAGTTTGATG ATAAGGAGTG |
| | GTATGTGGGT AAAGCTCAGA AGAAATCTGA GAGGGAACTT GAGTTGAGCC GGAGATATGA |
| | ACAAGGCTCA AGTGATGGTG GAAACAAATT TGATGGGTTG AATTTATATG TTAAGAACCT |
| | TGATGATACC GTCACCGATG AGAAGTTGCG CGAGTTGTTT GCCGAATTTG GTACAATCAC |
| | CTCTTGCAAG gtcagcattg tttgttttcc gcatacataa taacatgaga gatgcaattt |
| | tttttgtctc ttgattgatc ggaacctcat acttttgtaa caaacagGTT ATGCGGGACC |
| | CTAGTGGTAC TAGCAAAGGA TCAGGATTTG TTGCCTTCTC TGCTGCCAGT GAAGCTTCAA |
| | GAGTGgtaat ttaaataatc ctgtgtcaag acaatattaa atttgttttg agcctctatt |
| | ttctttcttg attcaatttc ttttggggtc ttctgcagCT GAATGAAATG AATGGTAAAA |
| | TGGTTGGTGG CAAACCGTTG TATGTTGCTC TTGCACAGAG GAAAGAAGAA AGGAGGGCTA |
| | AGCTGCAGgt agtacttccc accatagata aacaacccct acgtacactt atgtttgcta |
| | tgtctcaagt ccttatgttt ctttttcagG CACAGTTTTC TCAAATGAGA CCTGCTTTTA |
| | TCCCCGGTGT CGGTCCTCGA ATGCCAATAT TTACAGGTGG TGCTCCAGGT CTTGGACAAC |
| | AGATTTTTTA CGGTCAAGGA CCTCCACCAA TCATCCCTCA CCAGgtacca ttttgttcta |
| | actgaccact atgtaactct gcttgaatat gggactcttt caatcaataa gcactcactt |
| | ggttctactt aaatctgtga tatagCCTGG ATTTGGATAT CAGCCTCAGC TGGTTCCTGG |
| | AATGAGGCCG GCCTTTTTTG GTGGACCGAT GATGCAGCCA GGTCAGCAAG GTCCACGACC |
| | AGGTGGCAGA CGGTCAGGTG ATGGACCCAT GCGCCATCAG CATCAGCAGC CAATGCCTTA |
| | CATGCAGCCA CAGgttagtt tataaaaaaa ggagaatatg tcttaaatcc cagatcaaga |
| | tgaatctata agtctttgct ttcttctctc ctctagATGA TGCCAAGAGG ACGAGGGTAC |
| | CGGTACCCTT CTGGTGGTAG AAACATGCCT GACGGTCCAA TGCCAGGAGG AATGGTTCCA |
| | GTTGCTTATC ACATGAATGT AATGCCGTAT AGTCAGCCTA TGTCCGCTGG TCAATTGGCT |
| | ACTTCCCTTG CTAATGCTAC ACCTGCTCAA CAGAGAACAg taagtctctc tcaatacctc |
| | ttgacttgct gctatgtagg agaaaaaata agattactta cattcgatat gtttgttttg |
| | gggtttttgt agCTTCTTGG TGAGAGTCTA TATCCATTAG TGGACCAGAT AGAGAGTGAG |
| | CACGCTGCGA AAGTGACTGG TATGCTTCTG GAAATGGATC AGACCGAGGT TTTGCATCTG |
| | CTCGAGTCAC CAGAGGCTCT AAATGCCAAA GTTTCAGAGG CATTAGATGT GTTGAGAAAC |
| | GTGAATCAGC CATCTTCACA GGGAAGTGAA GGCAACAAAA GTGGAAGTCC AAGTGATCTC |
| | TTGGCTTCAC TTTCCATCAA TGATCATTTA TGAgaagctt ttgttcgagt tttttttttt |
| | actttgactc tcttcctctc tatctctctc tctgattgac aaatttttgc gggaatctat |
| | ttgctgtttt agactttttt tgctcgatat gattgtttct gttttgactt cttacttttt |
| | tgggttgact taaaaaagga tggttttatt ttattttgtt ggattatatt ttactgttgc |
| | aaaattttgc gctcagttta aaacttttta tgattgattt aagtttttag ttatttgttg |
| | gtaattgtca attttgaacg agaaggtgat gaaattagga tatgtatagt tcattagcta |
| | attaatccaa ttttagtttt tcacaaatat taacaactga ttataaatgt atcatttttt |
| | gtgattacca attttcataa ttctaaacca atagtaaatt actttgtagt aaaatcaaca |
| | caaactcatg gaccatgact cgtaaagaag ataaaaacaa gtggtacatt tat |
| 13 | atatcaacat caaacaatat tatagcaaag ataatgtgat tatttggtta ttgtaattga |
| | aattaatcca tataccaatt cattttgttt tgttatatat atcgagaggt tattgtgatt |
| | taaaaaaaaa aaatatttaa tcatctaccc agtaaaacta cgccacataa ccaccacaat |
| | aactctaaga gcacttctta ccttgaaacg tctcttactt aaattaataa ttaaatcttt |
| | aatttttatc atttattaac ctaagaaaca gctaataaat atttattaat ctaagagact |
| | tacacgtctc tctttcttat aacatatcaa catcaaacaa tattatagca aagataatgt |
| | gattatttag ttattgaaat tgaaattatc cacacaccaa ttcattttgt tttgttatat |
| | atatcgagag gcctaagaca acacttacac gtctatcttt ctttcctttg tataccaaaa |
| | aatataaaat aaaaaacact ATGGCGGAAA ACTACGACCG TGCCAGTGAG TTAAAAGCAT |
| | TCGACGAGAT GAAGATTGGC GTGAAAGGAC TCGTCGACGC CGGAGTCACA AAAGTCCCGC |
| | GCATTTTCCA TAACCCGCAT GTTAACGTAG CAAACCCTAA GCCTACATCG ACGGTGGTGA |
| | TGATTCCAAC AATCGATCTA GGTGGCGTGT TCGAATCCAC GGTCGTGCGA GAGAGTGTAG |
| | TTGCGAAGGT TAAAGACGCA ATGGAGAAGT TTGGATTTTT CCAGGCGATT AACCATGGGG |
| | TTCCACTTGA TGTGATGGAG AAGATGATAA ATGGTATTCG TCGGTTTCAC GACCAAGATC |
| | CAGAAGTGAG GAAAATGTTC TATACCCGAG ACAAAACCAA AAAGCTTAAA TATCACTCTA |
| | ATGCTGATCT CTATGAGTCT CCTGCTGCGA GTTGGAGAGA TACCTTAAGT TGTGTCATGG |
| | CTCCTGATGT TCCAAAAGCA CAGGACTTAC CTGAGGTTTG TGGgtaagaa tacatttctt |
| | taatttattt ctaatctaag aagaaacaag actagtttaa actttgattt gatattattg |
| | atgtggtttg aaaattggtt ggtgtgaata ttgttagGGA GATCATGTTG GAGTACTCAA |
| | AGGAAGTGAT GAAGTTAGCG GAGTTAATGT TTGAAATTTT ATCAGAAGCT TTAGGGTTGA |
| | GTCCTAACCA CCTCAAAGAA ATGGATTGCG CAAAAGGTTT ATGGATGCTC TGTCATTGTT |
| | TTCCACCCTG TCCTGAGCCA AACCGAACAT TCGGCGGCGC TCAGCACACA GACAGATCTT |
| | TCCTTACTAT TCTTCTTAAC GACAACAATG GAGGACTTCA AGTTCTCTAC GATGGATACT |
| | GGATCGATGT TCCTCCTAAT CCCGAAGCAC TTATCTTTAA CGTAGGAGAT TTCCTCCAGg |
| | caagtcgttg tttactcttg aattgaatgg tctataaaaa cccataagtc acaaaaagta |
| | agtctttttt tttttttttg cagCTTATCT CGAATGACAA GTTTGTAAGC ATGGAGCATA |
| | GAATTTTGGC AAATGGAGGT GAAGAGCCGC GCATTTCGGT CGCTTGTTTC TTTGTGCATA |
| | CTTTTACTTC ACCAAGTTCG AGAGTATATG GACCCATTAA AGAGCTTCTG TCTGAGCTAA |
| | ACCCTCCAAA ATACAGAGAC ACCACCTCGG AATCCTCCAA TCACTATGTG GCTAGAAAAC |
| | CTAATGGGAA TTCTTCGTTG GACCATTTAA GGATCTGAaa cttgaaccta tatctcagag |
| | gttttcttga gtttccaata aaatttggtg cacgctgtga cgtaccatgt tcaagacctt |
| | gaacgtatca ttcaataatt cttccgttgt gagtttcggc tgcatgtttg acccaaacca |
| | gagagagtat ggatcaatca aggagagtga acctaaaaat aaaaaaaaaa taaaaaaaag |
| | agtgtgaacc tttaattatg taaaatctta aataaacatc gagattgtat ttaaggattt |
| | tccatttgtt ataatctcaa tttaccttta atatgaggtt tatattcttt cttataacat |
| | atcaacatca aacaatatta tagcaaagat aatgtgatta tttagttatt gaaattgaaa |
| | ttatccacac accaattcat tttgttttgt tatatatatc gagaggccta agacaacact |
| | ttggcgtcta tctttctttc ctttgtatac caaatgtttg attttgttat ttaaatca |
| 14 | acgtacgatg cctgagctgc gtagcaacgc acgcagagat cgggataaga agaacccgaa |
| | gcagaaccca attgctttga aacaatcacc tgttaggaga aatccgaggc ggcagctgaa |
| | gaagaaagtg gtggtgaagg aagcgatcgt tgcagctgaa aagacgacgc ctttggtgaa |
| | agaggaagaa gaacagatta gggtttcgag tgaagataag aagatggatg agaacgacag |
| | tggtggtcaa gcagctccag tgcctgatga tgaaggaaac gctcctccac ttcctgaaaa |
| | ggtgtcaact ttattgttgg ttttgttgtt tttatgaggt tttagttcat cggaattgtc |
| | tcttgcattg tgtgttgtgt tttttgatta ggagaaagct ctcaaactta ggcatgccac |
| | ttaaagttaa aactttctct tgtaggatga tttgattatt gactccttgg tttttacagg |
| | ttcaggttgg taattcaccc ATGTACAAGT TAGATAGAAA GCTAGGCAAA GGTGGTTTTG |
| | GACAAGTTTA TGTTGGTCGA AAGATGGGCA CGAGTACTTC TAATGCTAGA TTTGGCCCGG |
| | GAGCTTTGGA Ggtatgctgt ttgtgtttgc aagtttactt gctttctttt ggttttctgt |
| | gatctgtaat gtgattttga tgtgtccact tttgtagGTG GCTTTGAAGT TTGAGCATAG |
| | AACCAGCAAA GGATGTAACT ATGGGCCACC GTATGAGTGG CAAGTTTACA Agtgagcgtt |
| | atggtctctt gtctttggct ctaggattca tcttctgctt gttcaaatag tttgtttata |
| | aaaggatgag ataactaatg atgctttatc atctgttcgt ccagTGCACT TGGTGGCAGT |
| | CATGGTGTGC CACGAGTTCA TTTTAAGGGT CGGCAGGGCG ATTTTTACGT GATGgtatgt |
| | ggaatttagt caggtctgaa caagagcact tgcagtatga tgaattactg tttttaatct |
| | ttcatacagG TTATGGATAT CCTTGGGCCT AGCTTATGGG ATGTTTGGAA TAGTACCACC |
| | CAGGCgtaaa cattcactct gagaaacatt tactttattt tgtagcatct gaagattttg |
| | ttatatgaac cattgataaa cataattttt cctgagatga gcccttcaat attggtggca |
| | ctcaccatat gatttgtgtg ttttatacat tccagGATGT CAACAGAGAT GGTTGCATGC |
| | ATTGCAATTG AGGCAATATC CATATTAGAA AAGATGCATT CTAGAGGgta attttctaat |
| | atttctgcta ctgtaactct ctttcttcaa gtggttttta tttgctaaga agcagtgctc |
| | ctgtttctac agATATGTGC ATGGCGATGT AAAACCAGAG AATTTTCTGC TTGGGCCTCC |
| | TGGAACTCCT GAAGAGAAAA AACTTTTCCT TGTAGACCTC GGCTTAGgta cactttattt |
| | ttgttataag agtgagcgta ctttattgtc tttctgctgc ttatccaatc tgttgatctt |
| | gcagCATCCA AATGGCGAGA TACTGCAACT GGACTACATG TTGAATATGA CCAGCGTCCT |
| | GATGTTTTTA Ggtaagttga ttcagctagg cataaagcct gtgagattga ttcttatcag |
| | ggacttcaac tttagggtac ttattaacgt gttggctttt tcattttcag AGGAACAGTA |
| | CGTTATGCTA GTGTACATGC TCATCTTGGC AGAACTTGCA GTCGGAGGGA TGACCTGGAA |
| | TCTCTTGCTT ACACTCTTGT TTTCCTTCTT CGAGGCCGGC TTCCATGGCA AGGGTACCAG |
| | GTTGGGGACA CTAAAgttat ttgttttatt tcctggcaac tttccttgtc aatcattaac |
| | ttggtctatt tgttagggag agAACAAAGG TTTCCTTGTT TGCAAGAAGA AGATGGCCAC |
| | TTCCCCAGAA ACTCTTTGCT GCTTCTGTCC CCAACCTTTT CGTCAGTTTG TCGAGTATGT |
| | GGTCAATTTG AAGTTTGATG AGGAGCCTGA TTATGCTAAA TATGTCTCCC TTTTTGATGG |
| | AATAGTCGGC CCAAACCCAG ACATTAGGCC AATAAATACT GAGGGTGCAC AGAAGGTGAT |
| | TTGGTGAtct tctttatgaa acatatattg aggtttacta tttagctccg gtctgaatgt |
| | ctaaagtttt ttcgtgtttg tctggtgtga agctcataca tcaagtgggt caaaagaggg |
| | ggaggctgac aatggacgag gaggatgaac aaccaacaaa gaagatcaga ttgggcatgc |
| | cagcaacaca atggatcagc atttacagtg ctcacagacc aatgaaacaa cggtgacatc |
| | ttggatcata cttgagaatt cttcggctgt acgttgatga ccatgcagct gacatgtctt |
| | ttatctttgt gcagatatca ttataatgtt actgatacaa ggcttgcaca acacattgaa |
| | aaaggaaatg aggatgggtt atttatcagc agtgtggctt cttgcacgga tctctgggct |
| | ttgatcatgg atgcaggaag tggctttacg gatcaagttt accagttatc accaagcttt |
| | ctccacaagg tagcttcatt taatatt |
| 15 | tgtctaactg catgtctatc atgtacatta agatcaagac taatataaaa ctcacaaatc |
| | aatatactac ttaagaaaaa gaaaaaaatc tggttctttt ttattcatgc acacacatag |
| | tataagttaa aaaatgacca tattaatttg taaactgacc aatcgtgtat ataaaaggac |
| | accttctcta cctacttata tattatacat catttctcta cattgttcac cagctctctc |
| | catctctcta ctccaagcat aagaggtaat ctctcaatag tttgaaacaa ccttttgtaa |
| | aacgtattgt aacttactta aaattgtaga acgtgagaaa tatcttaaat gtttaaagtc |
| | ttcctttttc acccaagaac tgaaaatgat tttgcatata tattttctca agtgggtata |
| | atggatataa agaaattata caatgactaa ggaacaaaat aaaatctctt ttattgaata |
| | atgatttgaa tcagttctcg ATGGCCCAAA GGTTGGAGGC AAAAGGCGGA AAGGGAGGGA |
| | ATCAATGGGA TGATGGAGCC GACCATGAAA ATGTAACAAA GATACATGTA CGAGGTGGTC |
| | TTGAAGGAAT CCAATTCATC AAGTTTGAGT ATGTCAAAGC TGGACAAACA GTTGTTGGAC |
| | CAATTCATGG TGTCTCGGGT AAAGGTTTCA CACAAACGgt aagcatgtta aatatagaac |
| | tacctgaact cttttttttt gaagatataa ggttgtatcc tggattgaat gtttagaaaa |
| | tttgaacaca gaaactaatc ggttgtgaag gtgatatgat gttaatagct agatgtacat |
| | gtatatcctt actatatata tcagaacttt ttagttggtc aacttttaat gatcggtgct |
| | taaattttat taattaatcg agtctccata attgttttaa attatccccc acagcttata |
| | tattactgat caagttttaa tattcttttt tttttcttac agTTTGAGAT TAATCATCTC |
| | AATGGCGAAC ATGTGGTGTC AGTAAAAGGT TGCTATGATA ACATATCCGG TGTGATCCAA |
| | GCACTTCAAT TCGAAACCAA TCAAAGGAGT TCTGAAGTCA TGGGATACGA TGACACTGGC |
| | ACTAAGTTTA CACTTGAAAT CAGTGGAAAC AAAATCACTG GGTTCCATGG ATCTGCTGAC |
| | GCAAACCTAA AATCTCTTGG AGCTTATTTC ACACCACCTC CTCCTATTAA ACAGGAATAC |
| | CAAGGTGGTA CTGGAGGCAG CCCATGGGAC CATGGTATTT ACACCGGCAT AAGAAAAGTC |
| | TATGTTACAT TTAGTCCCGT TAGCATATCG CATATCAAGG TCGACTACGA CAAAGATGGA |
| | AAAGTGGAAA CGCGTCAAGA CGGGGACATG CTTGGAGAAA ATAGGGTCCA AGGACAACCA |
| | AACGAGgttc tagttttaac actccttact tcttattatt ttagtttttt ttggtaaaat |
| | gctaaatctt taatagaaag gaatatgtca agagtaaatc atatatggga agaatcataa |
| | accattcgtt aacccttcaa ttttttaaaa tatataaatt gaaggatccc tttatttgtt |
| | ttttgcagTT TGTAGTGGAC TATCCATATG AATATATTAC ATCAATAGAA GTGACCTGTG |
| | ACAAAGTCTC TGGCAATACA AACCGAGTTA GGTCGTTGAG TTTCAAGACA TCAAAAGACA |
| | GAACATCTCC TACATATGGA CGTAAGAGCG AGCGAACTTT CGTGTTTGAG AGCAAAGGTA |
| | GGGCTCTTGT TGGGCTCCAT GGAAGGTGTT GTTGGCCTAT TGATGCTCTA GGTGCACATT |
| | TTGGTGCGCC TCCTATTCCT CCACCTCCTC CCACGGAGAA ACTACAAGGA TCAGGTGGTG |
| | ACGGAGGAAA ATCATGGGAC GATGGAGCTT TCGACGGTGT GAGAAAGATA TACGTGGGAC |
| | AAGGTGAGAA TGGTATCGCA TCTGTCAAGT TTGTGTATGA CAAGAACAAC CAGTTGGTAC |
| | TAGGAGAAGA GCATGGAAAG CATACTTTGC TTGGATACGA AGAGgtgatt aattatacta |
| | tacttcgttg ctattttctt aaactataac tataaagttg tgttattgtt attctgatga |
| | accgctttca cagTTCGAGT TGGACTATCC GAGTGAATAC ATCACAGCGG TAGAGGGTTA |
| | TTATGATAAA GTGTTTGGTA GTGAATCTTC AGTAATAGTC ATGCTTAAGT TCAAGACCAA |
| | TAAACGAACC TCCCCGCCTT ATGGAATGGA TGCTGGCGTT AGCTTCATAC TCGGGAAGGA |
| | AGGTCACAAA GTGGTAGGGT TCCATGGAAA AGCTAGTCCC GAGCTCTATC AGATTGGGGT |
| | CACTGTTGCC CCAATCACCA AGTGAcgacg tccttgaact ttattctcaa atcaagtttg |
| | atcatgcata tttgttaagg cgcctctctc gtattgtctc caccactttt ctacgtgttt |
| | tgttttctcc gatgttttac tttgaaaaat ctatttcaat caagcaatat cgtgtaataa |
| | aagcaaggtt ctcgaacctg cgggtaaact ttttattttg aataatttat tttcaatcaa |
| | gcattctttt gactttttgc tttaaccaaa tgtctctagt ttcaaaaaag attaagaact |
| | caaagatata agaattactt tcttattaag cttactttct tattaagctt aggaaaatta |
| | ctcaaaacgt aaacaatctc aaagtcttaa tttctctaaa ctcatatagt caaccacagc |
| | ttgggactca tatatataga gattaataaa ccaaaacata ctaggattag cattagataa |
| | ctcctaacat atatctttag atatctccta aagatttaac ataat |
| 16 | ttctaaggaa atgttttgtt aatatgaatt cattaactgc aacctaaaga aaagtttgtg |
| | aataactcag cgtgacctaa tcctacaaaa aaagtataat gttccactca gagtcactgg |
| | tcaaaaagta ttaattcttt aaaagaacct ctttttgtgt tgtataatga actagtttgg |
| | ttataaactt ataacttaaa gggacatggt tgttgactta aacttaggta gaattgtttt |
| | ttatatagaa atggagcaag tcgatcttaa atgttagatc ataaataaac ttctcatgaa |
| | acctaaaaga aaaaatatat aaacacccaa acccattcca ttcacttcaa caactcaatt |
| | acaattatgc ttatatatct tacatgcaaa acttcatcat tatcatcatc atctctagct |
| | cctcctttga atcttttcca aattcaactt ccgaaagaga taaccctaat ttctagtctt |
| | cttcttctaa attttcttcc ATGGATATCG AAAAGGCAGG GAGCAGAAGA GAAGAAGAAG |
| | AACCCATTGT TCAAAGGCCA AAGCTAGACA AAGGCAAAGG AAAGGCTCAT GTATTTGCTC |
| | CTCCTATGAA CTACAACCGG ATCATGGACA AACACAAGCA AGAAAAGATG AGCCCTGCCG |
| | GGTGGAAAAG AGGTGTAGCA ATCTTCGATT TTGTTCTTAG ACTCATCGCA GCAATCACAG |
| | CTATGGCTGC TGCAGCAAAG ATGGCGACAA CGGAAGAGAC TCTTCCTTTC TTCACTCAGT |
| | TCTTGCAGTT CCAAGCTGAC TACACTGATC TACCAACTAT GTCgtaagtt tctctccaaa |
| | tgttactctt actataggtt atgccaagaa tgtagtaacc aactatggaa atgaaacccc |
| | aaatgtgtat agtcgtacta tagataatac caagactgct acgtagctta acccgttgaa |
| | tccaaccaaa gccaggctag ttgcaaagtt caagcagtag ttagagagaa aaaatgagct |
| | acgttttaaa taagggggga aaaaaaacta tcaacatgaa tttcgagcaa tgtgcttggt |
| | gcttattagg gatttaatta tggtacatga ttttcaatta tataaagatt caaacttata |
| | tcattttttt ttattgtttt gttttgcagA TCTTTTGTGA TAGTAAACTC AATCGTGGGT |
| | GGCTACCTAA CCCTCTCATT GCCTTTTTCT ATAGTCTGTA TCCTCCGCCC CCTCGCGGTG |
| | CCGCCTAGGC TATTCCTGAT CTTATGTGAT ACGgtaacat ttataaaaaa aatttgaaaa |
| | taaatagtta taataatgca atgccaaaca tacaaatgaa atttctcatt ttgtttgtgg |
| | tttaacaatg aaacttttcg tagctttaaa aaaaagtaca aacgcaaacg ctaaaataag |
| | tcaaggcttt acttaagctc gagtaatcct tatattggtc acaaattaca atgaatatgt |
| | ttgttgagta aacatatgac aaatccctct aactagttcg tacggttgtg ttggtccagG |
| | TGATGATGGG CCTCACCCTC ATCCCCCCAT CCCCTTCCCC ACCCATACTT TACTTGGCGC |
| | ACAACGGGAA TTCAAGCTCG AACTGGCTTC CGGTTTGCCA GCAGTTTGGT GACTTTTGCC |
| | AAGGAACGAG CGGTGCCGTG GTGGCATCCT TTATTGCTGC GACTCTTGTC ATGTTCCTCG |
| | TCATCCTATC TGCATTTGCT CTCAAGAGAA CAACCTGAaa acttggattg atcctcttga |
| | ttaaattttt atgtgctttg atattcattt gtgtgaattt ttattaaaag gttcctatgt |
| | ataatttggt tttgttgtgt ttggtaactc gggttttagt gtggaaaaat gttgtaaatc |
| | aatcttctat attcacatat tgttttcttt ttccctatat aattttcgtt tcaaagataa |
| | caaattttaa acttatatct gcccggccat aattttaatt aaattagtaa gggtgttaag |
| | ttgatgtaat atcacatgat tttaaatatc taagtaacta actaattata |
| | tatcattata tttatatatt tgactaggtg gggctcaatt ggctccaaag aattttgttt |
| | gcatgcttaa ttattttgta tttggtggat gatttgattt gaaatgataa aagtttaatc |
| | cattgtcctt ccacctcttc tagcatttga tattttctcc tattaattgt ttaatatg |
| 17 | ttgtaataag taaattcggc cacctagttc tccggtgaaa gaaagaagaa gacacaaatg |
| | gagctccgtg acgtggaaaa acattattag gcccaaaacc ctctgactta aaaaagactt |
| | gataattgaa taaatagttt aatgtcgttg acataaacgt aagccgtctt agctcagtgg |
| | tagagcgcgt ggcttttaac cacgtggccg tgggttcgat ccccacagac ggcgttttcg |
| | tattccgaca taggttgtct tttttgctgc ttttctttaa ctgaaatatt ccgaccaatt |
| | ttttccagct gataagccca acggacaatg tgtaatattg cgattttata taaaagtttt |
| | gggccttttg attttccttg caataattaa cactcggtct tctccaacct aacaattatt |
| | ctagggtttt agagtttccg cacgaatcac gaatctctct ctctttcaca cacttcacac |
| | tttcaatata cactctcatt ATGACTACCG AAGAGAAAGA GATCCTCGCC GCCAAATTGG |
| | AAGAACAGAA GATCGATgta attgattact cttttattct ttacctatct atcatctctg |
| | tttatttgtt gttatttgtc ttttagtctg gaaatcatta gactgaattc agagtttttt |
| | aatctgttcc tgcccagatc tttgcttttg ttttgttttg tatatgcaaa tattggacct |
| | tattataaga ctttagatct gaatttacat gtaattaacc tttgtggatt ctctcatttt |
| | cccaattagt tcaattattg atgatttgtt gtagCTCGAT AAGCCCGAAG TTGAGGACGA |
| | TGATGATAAC GAAGACGATG ACTCTGATGA CGATGATAAG GATGATGACG AGGCTGATGg |
| | taaaagcttt ctacatttca ttcatcaaat tactggaata attagtatag ttcctagtat |
| | ttctgttagc ttacatctgg ggcagatttg ttgatgctca cgtgtatgtg tagatatgta |
| | gcaatgataa ttatatggcc atagcttgaa aatttagtga aaatgaatcc atcttctttg |
| | ttttcaaata atctttgcgt tgacttgtgt tgatagacat gtttgtggaa cttaatgtta |
| | tcatctattt tattcttgtt gattggtgat tggaaaacag GACTAGATGG AGAGGCAGGA |
| | GGTAAGTCAA AACAAAGCAG AAGTGAGAAG AAGAGTCGCA AAGCCATGCT CAAGCTTGGC |
| | ATGAAACCCA TCACTGGTGT TAGCCGAGTC ACCGTCAAAA AGAGCAAGAA Tgtttgtgtt |
| | ttctctttaa tattcagtca atcttaattt cttttattca cacatcaggc tttaatattg |
| | atctgttttg gggacatttg ctttggaaca cagATCTTGT TTGTCATATC AAAGCCTGAT |
| | GTGTTCAAGA GTCCAGCATC AGACACATAT GTGATCTTTG GAGAGGCGAA GATCGAGGAT |
| | TTGAGCTCTC AGATCCAGTC GCAAGCAGCA GAGCAATTCA AGGCACCAGA TCTCAGCAAT |
| | GTGATCTCAA AGGGTGAGTC ATCGAGCGCT GCAGTGGTTC AGGATGATGA GGAGGTTGAC |
| | GAGGAAGGTG TTGAGCCAAA GGACATTGAG TTGGTGATGA CTCAAGCAGG AGTGTCTAGG |
| | CCAAATGCTG TGAAGGCTCT CAAGGCTGCA GATGGAGATA TTGTCTCTGC CATCATGGAG |
| | CTTACCACCT AAaccaaagt cttttctact tagatgtggt ttaacctgag ttatgtgcca |
| | gagattgtcc aaagaattcg gaaatttttg gtttcaatgt ttttcatgaa gtgattttcg |
| | atgttgtatc agtataaacc tcataagttt ttgattttca gtttgatttt atattgaata |
| | tcaagtccaa gtgtttacca ttatagactt gtagttataa tttgtcaagt atcagtctgt |
| | ttaatgaacc gaacccaaag gatatggaca ccccttcact ccaaccaata cgaggtatca |
| | actgaggtta atcgatacat gcagtacaat gtacaaagtg ctacaagtgg aggttcatag |
| | actagaaaag tattcaacag gacctgattc taagagaaat tgttataaag ccgatgttta |
| | ttacctaact cctcaaggaa ggaggctagg gagttgcaag gaaggagctg gttttatcca |
| | agactacgaa agattcaaag gcacactgat ga |
| 18 | tcgatctgtg ttttgatttc tcgatcttga atctgttgga tcttgaatcc agtgagctga |
| | ttttgagtct tgttcagata tatttgatat tgcctagatt cagtttcggg tttctcaata |
| | tatttctcga ttgttaggtt tctatattga ttcaaatcga ttcatttgtg gcgagtttga |
| | ttgatttgag aatgtttgct ttccactatt ctaatggtta attgtgtaat tctttgcttc |
| | cttgactcac cttgtttgta gaagctacag atctgttgca gaaactatcc ttggactcgc |
| | cagcaaaagc ttcagagatc cctgagccta acaagaaggt gatttgcaga ttgaattttg |
| | gttttctgtt gtcacaacct ttgcttcttc cagttttttt taacgctttt gttttgtgtc |
| | ttgtgtagac tgccgtctac cagtatggag gcgttgatgt tcatggtcaa gttccttctt |
| | atgatcgatc tttgacacca ATGCTTCCCA GTGATGCTGC TGACCCTTCA GTTTGCTATG |
| | TTCCTAATCC TTACAATCCC TACCAGTATT ACAATGgtag cttcatcctc aaatcattta |
| | caatctagaa acattatttc actaaattgt caccactggt ttaacaagtt tttcgttttg |
| | taacttttca gTATATGGGA GTGGTCAAGA GTGGACTGAC TACCCAGCTT ACACAAATCC |
| | TGAGGGTGTT GACATGAATT CTgtaagtgt gtgctgacta gttataatag tgcctttcat |
| | cgtctttata ttttctttgc ttaacaggtt caatatttta ccagGGAATT TATGGAGAGA |
| | ATGGGACTGT TGTGTATCCT CAGGGTTATG GGTATGCAGC GTATCCTTAC TCGCCAGCAA |
| | CTAGCCCTGC TCCACAGCTT GGCGGGGAAG GGCAGTTGTA CGGTGCTCAG CAGTATCAGT |
| | ATCCTAACTA TTTTCCAAAC AGTGGACCGT ATGCTTCATC TGTGGCTACA CCTACCCAGC |
| | CGGATCTCTC TGCAAACAAA CCTGCTGGTG TGAAGACACT ACCTGCGGAT AGCAATAATG |
| | TTGCTTCTGC TGCTGGTATC ACAAAAGGAA GTAATGGATC AGCTCCAGTG AAACCAACTA |
| | ACCAGGCTAC CCTTAACACC TCAAGTAATT TGTATGGTAT GGGTGCTCCA GGAGGAGGTT |
| | TGGCTGCTGG TTATCAGGAC CCCAGGTATG CCTATGAAGG GTATTATGCT CCTGTGCCGT |
| | GGCACGATGG CTCTAAGTAC TCTGATGTGC AGAGACCTGT TTCTGGTAGT GGAGTTGCAT |
| | CCTCCTATTC TAAGTCTAGC ACAGTACCTT CATCGAGGAA TCAAAACTAC CGCTCAAATT |
| | CTCACTACAC Ggtatgatgt ctttccaaac ttctttttgc taatgaacac cattgtctgc |
| | tttactggca tatatatata gccgctcaag tcttccaaat ttgttaactg accttcaatc |
| | aacttttttc tttgcagAGC GTGCACCAGC CTTCATCAGT GACTGGCTAT GGTACAGCTC |
| | AGGGGTACTA CAACAGGATG TATCAGAACA AGTTATATGG TCAGTATGGT AGCACAGGGA |
| | GATCTGCTTT GGGTTATGCT TCATCTGGGT ATGATTCAAC AACAAATGGA AGAGGATGGG |
| | CGGCCACAGA CAACAAATAC AGAAGCTGGG GCAGGGGTAA CAGTTACTAT TACGGAAATG |
| | AGAACAATGT AGATGGTTTG AATGAACTTA ACAGGGGACC TAGAGCTAAG GGCACAAAGA |
| | ACCAGAAGGG AAATCTAGAT GATAGCTTAG AGGTTAAGGA GCAGACTGGA GAATCAAATG |
| | TAACTGAGGT TGGGGAGGCG GATAACACAT GTGTTGTTCC TGACAGAGAA CAGTACAATA |
| | AAGAAGATTT CCCAGTGGAT TATGCAAATG CCATGTTCTT TATCATCAAG TCATACAGTG |
| | AAGATGATGT GCACAAGAGC ATTAAATATA ATGTTTGGGC TAGCACACCA AATGGAAACA |
| | AGAAGCTTGC TGCAGCATAC CAGGAAGCTC AACAGAAAGC TGGCGGCTGT CCCATCTTTC |
| | TGTTTTTCTC Ggtgtgtata taatcctgaa attaaaaact gtgctctttt tactttgttt |
| | tatgatattg ttctttatac tccagttttt gtctttcagG TCAATGCAAG TGGACAATTT |
| | GTTGGTCTTG CTGAAATGAC AGGACCAGTT GATTTCAACA CAAATGTGGA GTACTGGCAG |
| | CAAGATAAGT GGACCGGCTC TTTCCCCCTC AAGTGGCATA TTGTGAAGGA TGTGCCAAAC |
| | AGTTTACTGA AGCATATTAC TTTAGAGAAC AATGAGAACA AACCTGTTAC CAACAGCAGA |
| | GACACACAAG AGgtaaatat ttgtgacatc ttttggcttg ttttactgat tactccacga |
| | gcgtttttgt tttcttgtgc ctaactttct ttgtttggat catattagGT TAAGTTGGAG |
| | CAAGGTTTGA AGATTGTGAA AATTTTCAAG GAGCATAGCA GCAAGACTTG CATTTTGGAT |
| | GATTTCTCAT TCTACGAGGT TCGACAGAAG ACTATCTTGG AGAAGAAAGC CAAGCAAACC |
| | CAGAAACAGg taagaactag aaaacaattt cagaaatctt tttcattcag tatatatata |
| | acttgagtgt ttctaatgta ttaaagctta acagGTAAGC GAGGAGAAGG TAACCGATGA |
| | AAAGAAGGAA TCTGCAACTG CAGAGTCAGC GAGCAAGGAA TCTCCTGCAG CTGTTCAAAC |
| | GTCCAGTGAT GTTAAGGTTG CTGAGAATGG GTCTGTTGCT AAACCAGTCA CAGGCGATGT |
| | GGTGGCAAAT GGTTGCTAAc taagaggatg gtgtcgctca cggcatgggc ataaaactga |
| | ctagagatga agatatgaac aatcccgttt aacgtttctc ttgagaagaa gattgccgtg |
| | agccttgaag catggaagga gctttagtac ctgagacgga tccgtttctt tgcccttaga |
| | agtttaaatc ccagttattt ttttttcaat cttttcttgt tttcattttt ccttttcttc |
| | aaaatcgcag tctcgttaca agtttatgtt gggtttcttt ttcattttct gttgttccta |
| | ccctgtaaaa atgcgcatag gacctactaa atcgtgggaa gaattagaga aaaggagata |
| | aaagcagggt gggattttgt tttttcatgt ctgttggatt tttaggcaga gttttctttt |
| | cttttggttt cttgctttgg tttcagactt gactctcttg agtcgtttag aatttgagat |
| | ggtcttttgc ctctctcgtc ttgtttctgt cattctcca |
| 19 | gagcgaggtc ttgtgtccag tttatgtttg aatcggtgat caaaacacaa tcctaaacag |
| | tgttagttaa tttaaaagct tcaatagcga aagacttact ttttgttttt ggtttctaca |
| | cttttataag tttactaatg cagaacttga tgaagctttt ttctgaattc attgattagt |
| | gaatatcatt atcttgttat tatcgtagac aaattgatat gagatcctta attatgatac |
| | caaataaaaa ccaccactaa agtgaaagaa aaaacaaagt caaagtaata tacaatatca |
| | tacaaatatc tgcaaaacgt ggaggaaaag aaaaatcgaa taattcgatg attctctcta |
| | tcaaagaaac gaaaaagtcg tattgaagtt ttgccatttg tttataaaag aagtggctgt |
| | tcaacgattc taaagtcatt tactttacca ttttgatctg ttgctctgtt tcactgtgcg |
| | tgatcgggaa gaagaagaaa ATGTTGGCGA TTTTCGACAA GAACGTGGCG AAAACACCCG |
| | AGGCTCTTCA GGGTCAAGAG GGTGGATCGG TTTGTGCTCT TAAAGATAGG TTCTTGCCGA |
| | ACCATTTCTC CTCTGTTTAT CCTGGTGCTG TCACCATCAA TCTCGGATCT TCTGGTTTCA |
| | TTGCTTGCTC TCTCGAGAAA CAGAACCCTC TTCTTCCCAG gttttgtaca atagtttatt |
| | cctcaggatg atgttttctt cttctgtcct agatatgaga gatttgctat cttaatgttt |
| | cactggcttg caaagatagt ttaggatatg tttcactgaa tctgagagat tgagatatcg |
| | atctgttgtt atgttttgat ggaataatga agttatatat ctactttgtt gtgatgttta |
| | aaatgtgttg aaactggaag gatgtgatta gataagtggt ggtgattttt tcaaaacaat |
| | tttgtgtgtg tgacagATTG TTTGCTGTGG TGGATGATAT GTTCTGCATA TTCCAAGGAC |
| | ATATAGAGAA CGTTCCAATT CTTAAGCAAC AATATGGACT AACCAAAACA GCTACAGAGG |
| | TTACCATTGT GATTGAAGCC TACAGAACTC TAAGAGATCG TGGTCCGTAT TCAGCTGAAC |
| | AAGTTGTTAG AGATTTTCAA GGCAAATTCG GGTTTATGCT CTATGACTGC TCCACACAAA |
| | ATGTCTTCCT TGCCGGGgta agtttgaatt ctgcttcttt actatttgac acttatttct |
| | gcatattgta atgctgaggt tattattatt atacgcgttt cagGATGTAG ATGGGAGTGT |
| | TCCTCTCTAC TGGGGAACCG ATGCTGAAGG ACATCTTGTT GTTTCTGATG ATGTTGAGAC |
| | TGTCAAGAAG GGTTGTGGTA AATCCTTTGC GCCATTCCCT AAAGgtatgt agcaagccgt |
| | ttttcgggtt ttgaagacat ctcactgttc tttgatctag tgcaaatatg aattaggatg |
| | tggttgtgtg tatgcataat gcagGATGTT TCTTTACCTC ATCTGGAGGT TTGAGGAGCT |
| | ATGAGCATCC ATCAAATGAG TTAAAGCCGG TACCAAGGGT AGACAGTTCG GGTGAGGTTT |
| | GCGGTGTAAC GTTTAAAGTG GATTCTGAGG CCAAGAAAGA AGCGATGCCT AGGGTTGGGA |
| | GTGTTCAGAA TTGGTCTAAA CAAATCTGAa ctagctgaaa aaggcttgtt ttatttttta |
| | cttgttggac tcctgtggct gtgttccaca gatttactct tttcctgata ttctcactgt |
| | agccattcta aggactaatg gtgctcttat tgctattgta cctgtacttg gtaacaagga |
| | agctaagaat aaaatatttt ataaacgtct aatgattcca gtgtatgcat atgatgtcat |
| | attgataaaa ccagagctgc aagaacatga gctccaacaa taacaattca taaacaacct |
| | ttggtaacaa aacaaaacct aaaactgtaa tgaaacataa tgacaggtct tagactctta |
| | gtaagagcct aaggttaaca ctgcctgcag atttctccac attctcttta cgcagaaacg |
| | cctcgggtaa gacttgagcc atccattttc agacctctgt tgtctgatgc tgctgctgca |
| | tagtcctgac tgttttccct tctccttgaa gtcatatcaa ggccaacac |
| 20 | ttgcttaaca ctcttaaatt attctcaagg aatctttcga ttgtgttctt aggattcaat |
| | tagtaataga cttgagtgtg tttgacatat ctattgggct tcgattgttt gttgcgttta |
| | catgttataa taggttttta tttcttggtt caaacgaaac caaaacttaa aagtaaatca |
| | tttttttcta ctgaattttg tttttgatgc ttttgatttc atttgatcac ttcaacttta |
| | gttccagggt cttgacgatt taattcaaaa agcaaaaaaa tcaataggaa acaaaaactc |
| | ataaaggact ttgacataca gatgggccca ttgtttatga ccaatcctta tactatatat |
| | gggccttatt agttaaacct aaggcccaaa gtcagattag ggttttcaga aagtgtacta |
| | taaattcttc ttctttaaac aacttcgtct agtggaacga cgacggcaca aaagcttcac |
| | cggagatcag agacgcgaaa ATGgtaaatt gtttcttctc tttcgatgtg attttggaat |
| | ttgtaaagtt cgttgacttt gaagaacaac aatacatggt tgattgattt attgtattgt |
| | ttttcagatc tatcataaaa gttttcaatc taaatgatgt ttgtattttg attaacctta |
| | aaagtctctt gattttgtat gtgtgtgagt gattcatttt tgattttatg aattttgaag |
| | GTGAACATTC CAAAGACAAA GAACACTTAC TGTAAGAACA AGGAATGCAA AAAGCATACT |
| | TTGCACAAGG TTACCCAATA CAAGAAGGGT AAAGACAGTC TTGCTGCTCA AGGAAAGCGT |
| | CGTTATGACC GTAAACAATC TGGTTATGGT GGTCAGACTA AGCCTGTCTT CCACAAAAAG |
| | gtaacattga ttatcatgca ttgattgttt tttcagtttg aattaggtct agttagttga |
| | aatgaggtag ttttaaggaa ccatttatag tagaattttg gaagtgagct gtgaggaaac |
| | agacattcca atagtctcag ttttggactg agatacatct tgtgaatctt gttaacagGC |
| | TAAGACCACG AAGAAGATTG TTTTGAGGCT TCAGTGTCAA AGCTGCAAGC ACTTTTCGCA |
| | GCGTCCTATC AAGgtgcata gaacatagat cagttcatta taccggattt gtaacttggt |
| | aatttgctta cattgtgttg gtttgttgtt tatttcagAG GTGCAAGCAT TTCGAGATCG |
| | GTGGTGACAA GAAGGGAAAG GGAACATCTC TGTTTTAAgt tggtttcatc ttattttctg |
| | cgatttttgt acttgctgga tttggaatcc atttgtttta gctctctcgt ataagattgt |
| | ctcatctttg cttgttaact ctatattttg aatcatcaag atatggtttt gctgttaatc |
| | attgaccttc gatatttttt tgccaatccg ttctctctac caacctaaga aaaaatcact |
| | aatatctcac attagagggt gcaaaatttg gaaggtctat atcattgtcc aattttctga |
| | gtcatacaaa ttctttcata tgattcattg aacaagacac tcatttactt ataaagcgca |
| | tttatatgtt cacatgattt gtacaaaact catgagactg catcaagcag aaagtattta |
| | tttatcttta catgtcaaag ctttgagaat taagcaatga cgaataccct aagttcacct |
| | ctgtccccgc gagttatgcg catggtatca tcaacatagg taacttcgaa atccccag |
| 21 | gacgccctat ctttgggttg aaaacttgag tttccttagc agcttttgtg atattttgaa |
| | tcatttttat gggatatgtt tgagttattt tgtttttacg atatggtatt ggtaatacat |
| | actagttact acatagtcgt agactttcat gtttatttac aaatggatac aggtttaaaa |
| | acatttactt gcgactattt gatacacgtt agttacctgt taaaccagat taaataaaac |
| | taaaccactt gcacttgtta attgttagtg cttcgttagt tgtaaagctg agtaattttg |
| | tttccactcg agagagagaa aatggatctt atcttctttt ttttttttat catcacatcg |
| | atcgagaagc ctagagttag ggcctagggg tccactctca tattaataac ataaatgatt |
| | tcttgtgtga tatagcttca ctgatttatc agatcttttt gcatttgggt cgacaaacaa |
| | gaaagaagaa gaaagcttca ATGGAGAAGA GTAATGGCCT TCGAGTGATT CTGTTTCCAC |
| | TTCCATTACA AGGCTGCATC AACCCCATGA TTCAGCTCGC CAAGATCCTC CACTCAAGAG |
| | GTTTCTCCAT CACTGTGATC CACACGTGCT TCAACGCGCC AAAAGCTTCA AGCCATCCTC |
| | TCTTCACCTT CTTAGAGATC CCAGATGGCT TGTCCGAAAC AGAGAAAAGA ACTAACAATA |
| | CCAAACTTCT CCTAACGCTT CTCAACCGGA ACTGTGAGTC TCCGTTTCGT GAATGTTTGA |
| | GTAAACTGTT GCAGTCTGCA GATTCAGAAA CAGGGGAAGA GAAACAGAGG ATTAGCTGTT |
| | TGATCGCTGA TTCTGGATGG ATGTTCACAC AACCCATTGC TCAGAGTTTG AAACTCCCAA |
| | TATTGGTCCT CAGTGTGTTT ACAGTCTCCT TCTTTCGCTG CCAATTTGTT CTTCCTAAGC |
| | TTCGGCGTGA AGTGTATCTT CCACTTCAAG gtattgttat ttcttacatt tttcgtatag |
| | accaagcaac tcgttaacct aaaaacatat atctaaattt tctcacagAT TCAGAACAGG |
| | AGGATCTAGT TCAAGAGTTT CCGCCGCTTC GAAAGAAGGA TATTGTACGT ATTCTTGATG |
| | TAGAAACAGA TATACTAGAT CCATTCTTGG ACAAAGTTCT ACAAATGACA AAGGCGTCTT |
| | CAGGTCTTAT ATTCATGTCA TGTGAAGAGT TGGACCACGA CTCAGTGAGT CAGGCACGTG |
| | AAGATTTCAA AATTCCTATC TTTGGGATTG GACCATCTCA CAGCCACTTT CCAGCTACCT |
| | CTAGTAGCTT GTCCACACCC GACGAGACTT GCATTCCATG GTTAGACAAA CAAGAAGACA |
| | AATCCGTGAT TTACGTCAGT TACGGGAGCA TCGTGACCAT CAGCGAATCA GATTTAATAG |
| | AGATTGCTTG GGGTCTAAGA AACAGCGACC AACCCTTCTT GTTGGTCGTA CGGGTTGGTT |
| | CAGTCCGTGG CAGAGAATGG ATCGAGACAA TCCCGGAAGA GATCATGGAA AAGCTTAATG |
| | AGAAGGGAAA GATAGTGAAA TGGGCTCCGC AACAAGACGT TCTAAAGCAT CGAGCCATTG |
| | GGGGATTCCT GACACATAAT GGTTGGAGCT CGACTGTTGA GAGTGTTTGT GAAGCAGTCC |
| | CTATGATCTG TTTGCCTTTT CGTTGGGACC AAATGCTAAA TGCAAGATTT GTTAGCGATG |
| | TATGGATGGT CGGGATAAAC CTAGAGGATC GGGTTGAAAG GAATGAGATC GAGGGAGCGA |
| | TAAGGAGATT ATTGGTGGAA CCTGAAGGAG AAGCCATCCG AGAGAGGATA GAACATCTTA |
| | AGGAGAAAGT AGGACGATCG TTTCAACAAA ACGGTTCCGC ATATCAATCG TTACAAAATT |
| | TGATTGATTA TATATCATCT TTTTAGccac tgacatgttg tttctttgtg ttttaagttt |
| | ttcaaccgat aaattgtttg tgtatcagaa atttcttcct ttgtgtgttt tgtattgtta |
| | gaataaaatt ttcttcgtaa gttggaattt acatatatac ttaccactta attatcagcc |
| | acgttttcag caacttttta ctattatttt gcaacctact aatacaaacg catcttgtct |
| | ttttatgtcc cttaactaat gaaaatcaaa tataaattag accactagtt acatgcccta |
| | gagggaaaac gaatctggtc tttctttatt agcacatcat gaagagtata gttttgtctc |
| | actctcgagt aataaagaat gcgaagtgct aataaagaaa gaccagattc ggaaatttct |
| | ttatgttata tatagatgtt tgttatcaaa agggaaagaa ttacaccatt cactgaaata |
| | tcaggagatt tacatttgga aagaaggtca aaaggagaaa gcttca |
| 22 | tattgttgat tctctatgcc gatttcgcta gatctgttta gcatgcgttg tggttttatg |
| | agaaaatctt tgttttgggg gttgcttgtt atgtgattcg atccgtgctt gttggatcga |
| | tctgagctaa ttcttaaggt ttatgtgtta gatctatgga gtttgaggat tcttctcgct |
| | tctgtcgatc tctcgctgtt atttttgttt ttttcagtga agtgaagttg tttagttcga |
| | aatgacttcg tgtatgctcg attgatctgg ttttaatctt cgatctgtta ggtgttgatg |
| | tttacaagtg aattctagtg ttttctcttt gagatctgtg aagtttgaac ctagttttct |
| | caataatcaa catatgaagc gatgtttgag tttcaataaa cgctgctaat cttcgaaact |
| | aagttgtgat ctgattcgtg tttacttcat gagcttatcc aattcatttc ggtttcattt |
| | tacttttttt ttagtgaaaa ATGGCCGATG GTGAGGATAT TCAGCCACTT GTCTGTGACA |
| | ATGGAACTGG AATGGTGAAG gtgagttaga ctgtttattt agatactgta tggttctaac |
| | cttctttgtt gtacatgtgt aagactactg atcatgattt ttgtatatta acagGCTGGT |
| | TTTGCTGGTG ATGATGCCCC GAGAGCAGTG TTCCCAAGTA TTGTTGGTCG TCCTAGGCAC |
| | ACTGGTGTCA TGGTTGGTAT GGGTCAGAAA GATGCTTACG TTGGTGATGA AGCTCAGTCC |
| | AAGAGAGGTA TCCTCACTCT GAAGTATCCA ATCGAACATG GTATTGTAAG TAACTGGGAT |
| | GACATGGAAA AGATATGGCA TCACACTTTC TACAACGAGC TTCGTGTTGC CCCTGAGGAG |
| | CACCCAGTTC TACTCACAGA GGCACCTCTT AACCCTAAAG CTAACAGGGA GAAGATGACT |
| | CAGATCATGT TTGAGACATT CAATGTCCCT GCCATGTATG TTGCCATTCA GGCCGTTCTT |
| | TCTCTCTATG CCAGTGGTCG TACAACCGgt tagttcttaa ctctaaacat ccaagtctga |
| | gttatattat cttcttactt gtatttactt aaagtcgttc tctttttgta acagGTATTG |
| | TGCTCGATTC TGGTGATGGT GTGTCTCACA CTGTGCCAAT CTACGAGGGG TATGCTCTTC |
| | CTCATGCTAT CCTTCGTCTT GATCTTGCGG GTCGGGATCT CACAGACTCA CTCATGAAGA |
| | TTCTCACTGA GAGAGGTTAC ATGTTCACCA CTACCGCAGA ACGGGAAATT GTCCGTGACA |
| | TAAAGGAGAA ACTTGCTTAT GTCGCTCTTG ACTACGAGCA AGAGCTAGAG ACAGCCAAGA |
| | GCAGTTCTTC AGTGGAGAAG AACTACGAGC TACCTGATGG ACAAGTCATA ACCATCGGAG |
| | CTGAGAGATT CCGTTGTCCT GAGGTTCTGT TCCAGCCATC GCTCATCGGA ATGGAAGCTC |
| | CTGGAATCCA TGAAACAACT TACAACTCCA TCATGAAATG TGATGTCGAT ATCAGGAAGG |
| | ATCTCTATGG AAACATCGTT CTCAGTGGTG GTTCCACCAT GTTCCCAGGA ATTGCTGACC |
| | GTATGAGCAA AGAGATCACC GCTCTTGCAC CTAGCAGCAT GAAGATCAAG GTGGTTGCAC |
| | CGCCAGAGAG AAAATACAGT GTCTGGATCG GAGGATCAAT CCTTGCATCC CTCAGCACCT |
| | TCCAACAGgt aaaaatccca attccgcctc tttaaaactt tcagctccat ttatgaaaca |
| | tgagtgaaaa tactgaaatt ttgttttgtt tgtgtgtgtg aatcagATGT GGATTTCAAA |
| | GAGTGAGTAC GATGAGTCAG GTCCATCGAT TGTTCACAGG AAATGCTTCT AAgtgtgtct |
| | tgtcttatct ggttcgtggt ggtgagtttg ttacaaaaaa atctattttc cctagttgag |
| | atgggaattg aactatctgt tgttatgtgg attttatttt cttttttctc tttagaacct |
| | tatggttgtg tcaagaagtc ttgtgtactt tagttttata tctctgtttt atctcttcta |
| | ttttctttag gatgcttgtg atgatgctgt ttttttttgt ccctaagcaa aaaaatatca |
| | tattatattt ggtccttggt tcattttttt ggtttttttt tgtcttcaca tataaatatt |
| | gtttgaatgt cttcaatctt ttatttgtat gagacaatta tttaagtatc gggtgacaat |
| | gcagctatta tgtattgtcg atttggatat tggcgcccaa aatatatact tagcctaaga |
| | atttggtaag tgagtggctt atgttttact ccagcaaaaa ttgtgtgtgt attaccattc |
| | tgatgcgaaa ca |
| 23 | aaaccatcta atctaagtct tgtctccttt atctacatat acggacaatt agatatcaca |
| | tgtacgaata tacaggcaat gtgggacaaa attcaaaaaa atgtgtctaa aaggggacaa |
| | gtggtcatta accttaattt aaattacggc caaatgttta gtaactaaat aaatatgggg |
| | tcgaaatgta aattctaaat tatctcacaa agtggggtac agaagtgaac actaataagt |
| | cataaagaga gatttaaagg agaaacgaaa agcattaaga tttaatttat atgaaattag |
| | tgaaaaccaa ccaaaaagaa tttatatgaa attctaaggg gcaaattgcg gaacaaagat |
| | tgtaaatagc aaaaggagtt tcagtataaa tatatgggga caagggccat aaaaataaca |
| | aaaacattct tagagagctt tggagataac gagaacaaga aagaaagaga agattatata |
| | catagaaaag gagagatcaa ATGGAGTGGG AGAAATGGTA CTTAGATGCG GTTCTTGTGC |
| | CAAGTGCTTT ACTTATGATG TTTGGTTACC ACATCTATTT GTGGTATAAG GTTCGAACCG |
| | ATCCTTTCTG CACCATTGTT GGTACAAATT CCCGCGCCCG TCGATCTTGG GTAGCAGCCA |
| | TCATGAAGgt agttatatta ctcaaaaacg atatatatcc cgaaataatc tttcaaaaat |
| | cttgtgttaa gtgattgtag taactagtaa gtagtaatta ctaattaatc atcatattag |
| | cgaaagtaat tagcttcatt gaacatatat accataatgt ttactaactg caatttttct |
| | atgaaaattg cttatgcaaa aacttagtat aggtgtcggc ccaaaatttt attaagtccg |
| | tatgaataca aaataaataa atttgcatgc atatttggcc aataagagac tataaatcca |
| | tacaatgtca taatatctct atgtatacat cattaacttt cttcatatat atgtacacag |
| | tatatacata gaattacttc tcaaatagta acaatatact gtgtctttgt tcagGACAAC |
| | GAGAAGAAGA ACATCTTAGC GGTACAAACA CTACGAAACA CGATAATGGG AGGGACGTTA |
| | ATGGCAACCA CTTGCATCCT CCTCTGCGCA GGTCTCGCTG CCGTTTTAAG CAGTACTTAT |
| | AGCATCAAGA AACCTTTAAA CGACGCCGTA TATGGAGCTC ATGGTGACTT CACTGTTGCA |
| | CTCAAATACG TAACCATCCT CACAATCTTC CTCTTCGCCT TCTTCTCTCA TTCTCTCTCC |
| | ATTCGCTTCA TCAACCAAGT CAACATCCTT ATTAACGCTC CTCAAGAACC TTTTTCTGAT |
| | GATTTCGGCG AAATAGGAAG CTTTGTGACT CCCGAGTATG TCTCTGAACT ACTCGAGAAA |
| | GCTTTCTTGC TCAATACGGT AGGTAATAGG CTGTTCTACA TGGGCTTGCC TTTGATGCTA |
| | TGGATCTTTG GGCCTGTGCT TGTGTTCTTG AGCTCTGCTT TGATAATCCC TGTTCTTTAT |
| | AACCTCGACT TCGTGTTTTT GTTGAGCAAT AAGGAGAAGG GTAAAGTCGA TTGCAATGGA |
| | GGTTGTGATG ACAACTTCTC GCCTTAAtta tctgttgatg ttgaattcga ataatgataa |
| | agctgtttgt tattactgat ttactagtct aaaaagtctt tcgatttact cttttcaaag |
| | cttaccaaaa aaaaaatgta ctagatccga gtcttttttt aatttttaat tttttttcct |
| | ggtgaagata ttcatgatct gctatatata attagtaaaa gttccatgga tagtcaaaat |
| | ggaaattaat taacaaaact atctttttta taaaattttt tattactatg ctgctaacaa |
| | gtaacaatga tgcgaccatc cttagtccct tacacttgat tcgtctatta ttttttctaa |
| | ttcaaatgtc aattttttaa tggcacagat actcgttttc aagtcaatgg agtgatactc |
| | atctgaattg gtcgtgtctt tttcctttat attagcccta tcagcggctt taataattat |
| | aacagacatt attatattga tgattattgg gatccaatga agaaagc |
| 24 | cattgttatt aagggaaatg aaatatctta actaaaccaa tttgttatct attgtgctct |
| | actgttctgt tcgtattgac tcgaacccac taaaccaaga cgagccctga ccgtcattgt |
| | ctaaattgac tcgaacccac taaagaaaaa aagaaaaaaa aacttagata ataattggcg |
| | cagaagggcc gattaataaa aactttaggc ccattaaagt aaagcttatt gtcaacccta |
| | tccagtctcc ttgtatatat ttatttacga caccaacgcg gcgttggtga ttcattctct |
| | tcagtcagag atttcgaaac cctagtcgat ttcgagatcc aaccaactct gctccttatc |
| | tcaggtaaaa ttctcgctcg agaactcaat tgcttatcca aagttccaat ggaagatgct |
| | ttcctactga atcttaggtt aatgttttgg atttggaatc ttacccgaaa tttctctgca |
| | gcttgttgaa tttgcgaagt ATGGGAGACG CTAGAGACAA CGAAGCCTAC GAGGAGGAGC |
| | TCTTGGACTA TGAAGAAGAA GACGAGAAGG TCCCAGATTC TGGAAACAAA GTTAACGGTG |
| | AAGCCGTGAA AAAgtgagtt ttatgatttc ctcgatctgt ttcatgagat agtggatgtt |
| | taaatttagg gttttcttag attactgctt gataacaacc gactaagttc ttcaattatc |
| | tatgtgtttg gttagttgct taactttatg acaattgact aagttcttca atgctaaaat |
| | tcctggaacc tacccaatat tagacggtca tgtgtttatc atcttgtatt ttctctttgt |
| | gacagAGGGT ACGTGGGAAT ACACAGTTCT GGATTCAGAG ACTTCCTTTT AAAACCGGAG |
| | CTTCTCAGAG CTATTGTTGA CTCTGGATTT GAACATCCAT CTGAAGgtta ttacaatgaa |
| | atacagcgta gctttgactt ttctgccttg cctttcacca ttctattacc gaatgatatt |
| | gtataattta cagaagtgac ttctccataa gatgttttag ttgtccggaa acttttaatt |
| | atatgtactt cgtctagttt tgagaagata tgttggttaa agatatttta tactttatct |
| | tggtcctttg cttatcatct aactaaatta aaaaaagttt gtgttgaggt caaattcttt |
| | tttatttcct gttataatgg tttttgtttt ctttgtttat taacgtttca ctgattactt |
| | tttccaggta ataaacgata tttcaatcta ttggtttgga gtgagcttaa acatgtgcta |
| | aagccaccaa tttaaaagat atggaggtta tcatctactt ataaaggctt tcttcggtac |
| | aattttcttt ggttctccac cagTGCAACA TGAATGTATC CCTCAAGCTA TCTTGGGCAT |
| | GGATGTCATC TGCCAAGCAA AGTCTGGTAT GGGGAAGACT GCTGTGTTTG TCCTGTCTAC |
| | TCTACAACAG ATTGAACCAT CTCCTGGCCA GGTTTCTGCA CTTGTCTTGT GCCATACAAG |
| | AGAGCTAGCT TACCAGgtat gaccttcttg tttcactcag gttcttggct tatagttttg |
| | ttgtacgtct tcttcctcta atgctttttg ccttgatgct gacaattact tgcagATCTG |
| | CAATGAGTTT GTGCGATTCA GTACCTATCT GCCTGATACA AAGGTTTCGG TGTTCTATGG |
| | TGGAGTCAAC ATTAAAATTC ACAAAGACTT GCTGAAGAAT GAATGTCCTC ACATTGTTGT |
| | TGGTACCCCT GGTCGGGTGC TTGCACTTGC CAGGGAGAAA GATCTCTCTT TGAAGAATGT |
| | GAGGCATTTT ATTCTTGATG AATGTGATAA AATGCTCGAG TCACTTGgta tgctgatttc |
| | tgacatcatt attacatcga tccctgaata attttatgtt ttaacacttt aacttttttt |
| | ttaccagACA TGCGAAGGGA TGTGCAGGAG ATTTTCAAGA TGACTCCTCA TGACAAACAA |
| | GTAATGATGT TCTCAGCAAC GCTCAGCAAA GAGATACGCC CAGTCTGCAA AAAATTTATG |
| | CAAGATgtaa tgttccatgg ccaattctct ctccctttgc aagtcttcta gttttcaact |
| | atttttagcc ttctatgagt gatcatagca ttagttgagc gtcttctgcg gttctgccct |
| | ggaaaagcgg caactgatct ctcaatgggt ctcaatccaa taatggttgg gtagtttgta |
| | gggaacgaga actgtgagtg tgagactctg tagctttggt atggtttcta tgggtgatta |
| | tagcattatt tgggcatctt ctgcggttct gccctggaaa agctgcaact gatctctcga |
| | tgggtctcaa tccactaatg ctttgggtag tttgtaggga tcgagaactg tgagtgtgag |
| | cctctgtagc attggtatga atgagtgacc attgcacaac aggatcttct ttcgtcatta |
| | ccttttattc agtttcaatt tctttgcaat tctagcagtg ctgggtgggt tttgggtggg |
| | gtactgtgtt gtcccaaggt ttcattgtga ttgtatgggc cttaatgttc cgagcaatat |
| | cgctgtatca tagcaaaact cacatctatg aagagaacct ggtggacgag gatctcagat |
| | caggggtttt acatccatct tcacttttgt agtgtaaatc atttcctgag aaaagcttgc |
| | taattattac ctgatatcta ttcctttcag CCAATGGAAA TATATGTCGA TGATGAAGCC |
| | AAGTTGACTC TTCATGGGCT TGTCCAGgta ctcttatctg gtgttaggtc ttcttattca |
| | atggaaatat agtttgttgt ttgatactta aaagaccttt tactgtcata ctgtaacagC |
| | ACTATATCAA ACTGAGCGAG ATGGAGAAAA CCCGGAAGTT GAATGACCTT CTTGATGCGT |
| | TGGACTTCAA TCAAGTTGTC ATTTTTGTGA AGAGCGTGAG CAGGGCTGCT GAGCTGAACA |
| | AGTTACTGGT GGAATGCAAT TTCCCCTCAA TATGCATCCA CTCTGGAATG TCTCAAGAAG |
| | AGAGgtctgt acattctctt caaaattcaa tgtttttgaa ggaccctacc tgctcttaaa |
| | gccctcatgg agaggagtcc aattcttaag gctaatacga tatgttatgt agGTTGACTC |
| | GATACAAAAG TTTCAAGGAA GGGCACAAAA GGATCCTTGT GGCGACTGAC TTGGTAGGAA |
| | GAGGGATTGA CATTGAGCGT GTCAACATTG TCATCAACTA TGACATGCCA GATTCTGCTG |
| | ATACCTATCT TCACAGGgta agtacataat actgaaattt attatttgat tgttgatctc |
| | actgaaaggg ctcttgtaac tttaccgttt tgctgtgtat ggtatagGTT GGCAGAGCTG |
| | GTAGATTTGG AACCAAGGGT CTTGCAATCA CATTTGTTGC ATCTGCTTCA GATTCAGAGG |
| | TTCTTAACCA Ggtatggtgt tcaatctttg taataagtcc acggaaaact cctcttgaaa |
| | ttgagttgga tatttagtaa agtggcaatt ataaatcttg gacagGTACA AGAGAGGTTT |
| | GAGGTTGATA TAAAGGAACT TCCGGAGCAG ATTGATACTT CAACCTACAG TAAGTGTGAA |
| | ATCCCTTACC AATTGTTTGT TTAAaagctt ggttttgtct ggttgtgata ttaatgttgt |
| | ttcttcttct ttctttgttc agtgccttct taaacaagta gcacgtccct caggaaagaa |
| | gctcttcaga tttcaacctt gtaggtgttc aaagggtcat gggggttcac aactatctct |
| | cgctccgttt gttttagtgt tttctatgac gacatttttt tccatatgtt tagaacgtct |
| | gttgtactct ttaaaggaga ttcgagtcac tctccaaatc gcacagttaa aagctgtcca |
| | gttttttgta caagagatta ttatgtttga aatatcagga tttagtctcg acctgattac |
| | tgtgttcctt aggaatcgat ctattatcaa tttatcatgg tgttgctaag aatcgtcatt |
| | catcagcgtt acttccttca tgtgatgctt tttttttata acacatttca tttagtgtgg |
| | aagagataca acacgtatat atggttactt tatatattga aaag |
| 25 | cttgtaagtt gttttccttt tgggatatgg gaagtgactt ctccgaccct tgcaaactaa |
| | caatggccat tacacactaa ttacaagcca aatttcctca ctaagcaacc tctcgtgttt |
| | atcataagac accgctctat ctcttattat tttattcatt gttttctaat ttcagactga |
| | ttaatcatac attagagaaa gtttattaaa accatctgat gtaaaaaatc acatttatct |
| | aaattaaata aatttgttat ctagtatata actatttatt gttttaacat ttggataaat |
| | tgtaagaaat tagaatgtaa aataagacag aaaatggtca actatgagca tctatcgcca |
| | tcatgatata gtttcgtcgt ttgcgttccc gacctaactc aaaacttcac caaccccatt |
| | tttaagcccc tttctttgtt tttatcctcc gatcgatcaa accaagaaaa aacactttcg |
| | tatttccctc gacgaaaaaa ATGGCAACCA TTTCGAATCT CGCTAATCTT CCCCGCGCCA |
| | CCTGCGTCGA CTCCAAATCT TCTTCCTCTT CCTCCGTCTT ACCTAGATCC TTCGTCAATT |
| | TCCGCGCTTT GAATGCAAAG CTTTCCTCTT CTCAGCTTTC TCTTCGTTAT AACCAACGAT |
| | CAATACCTTC CCTCTCgtaa gtctttatat ccatttgatg catgtctttt gtctctgttt |
| | ctcgctcttg gggttcacca aaaattgaat ctttttagct ggaaacgtac cacgaatctc |
| | aaagtaacat tttttataag atggattagg aaaagcaact gtatttcccc tttttggttg |
| | gtaaaagtct gatttttttg tttaatttgc agTGTGAGGT GTTCAGTGTC TGGTGGAAAT |
| | GGAACTGCTG GAAAGAGAAC GACTCTTCAT GATCTATATG AGAAGGAAGG TCAGAGTCCT |
| | TGGTATGATA ATCTTTGCCG TCCAGTCACA GATCTTCTCC CGTTGATTGC TCGTGGTGTT |
| | AGAGGTGTTA CTAGCAACCC TGCGgtaatt ttatcatctc tctttgtgtg tttggttttg |
| | cttttgctct gtgtttgttc atttgtcttt acttcttcac tttttataca tttgcagATC |
| | TTCCAAAAAG CCATTTCCAC TTCAAATGCT TATAATGATC AATTCAGgta tctttttgtg |
| | attgtcttag acttgtggtt gttaacaaca tgctattaaa actttagagt tcttctttat |
| | atgaaaagtt gtctgatatg ttaatggtat acctgacatg cactattagG ACACTTGTGG |
| | AATCGGGAAA GGACATTGAA AGTGCGTATT GGGAACTTGT GGTGAAGGAT ATTCAGGATG |
| | CCTGCAAACT TTTTGAGCCA ATCTATGACC AGACAGAAGG TGCGGATGGC TATGTCTCTG |
| | TTGAAGTTTC ACCTAGGCTT GCTGATGATA CCCAAGGAAC TGTTGAAGCT GCTAAATATC |
| | TTAGCAAGGT TGTCAACCGT CGTAATGTCT ACATTAAGAT TCCTGCTACT GCTCCATGCA |
| | TTCCTTCCAT CAGGGATGTC ATTGCAGCTG GAATAAGTGT CAATGTCACG gtaagttatc |
| | ctagtatgtt tcattattca agtttcttat tgcaagtttt aaagaacttc aaaataaaat |
| | aagtcataat acttcaaatt catgtattgt gtgatgatgt gctagatcac tggatttctt |
| | gggcgtttta aacctgaaac tagattagtt caagggtgtt ccaaggatgc actgatgtta |
| | ccttttctaa atcgtttctc atatgttctg ttctgtttca gCTTATATTC TCAATCGCCA |
| | GATATGAAGC AGTGATCGAT GCATATTTGG ATGGCCTCGA GGCGTCTGGA CTTGATGACC |
| | TCTCAAGAGT TACCAGTGTT GCTTCCTTCT TTGTCAGTCG GGTGGATACT CTCATGGACA |
| | AGATGCTTGA GCAAATTGGT ACCCCTGAAG CCTTAGATCT CCGTGGGAAG gtaaagctct |
| | attcatcgct gagatcttac accagccact gtgagtagag tattagctta tgacacatga |
| | tatgtttact cttgcagGCG GCTGTGGCTC AAGCTGCATT AGCATACAAG CTATACCAGC |
| | AGAAATTCTC TGGCCCAAGA TGGGAAGCTC TGGTAAAGAA AGGTGCCAAG AAACAGAGAC |
| | TTCTCTGGGC ATCAACAAGT GTAAAGAACC CAGCTTACTC TGACACCTTA TATGTCGCTC |
| | CTCTCATCGG ACCTGACACT gtaagtcatc tttttgtttg tgttgaagtc aataggctgt |
| | attaacgctt tggaagtata ttcatagttt ttgtgggtgt gatttagGTA TCAACCATGC |
| | CGGATCAAGC CCTGGAAGCA TTCGCAGATC ATGGAATAGT GAAGAGGACA ATAGATGCGA |
| | ATGTGTCAGA AGCAGAAGGG ATTTACAGTG CACTAGAGAA GCTGGGAATA GACTGGAACA |
| | AAGTAGGAGA ACAGTTGGAA GACGAAGGAG TAGATTCCTT CAAGAAGAGT TTCGAGAGTC |
| | TGCTCGGTAC ACTGCAAGAC AAGGCCAACA CTCTCAAACT AGCCAGCCAT TGAggaaatg |
| | agtcatcatt atgtttttgg ttacgctaaa ataaaaagaa gaacctttgg cttttgttct |
| | tcaatcctta tgcatgcttt ctaaagtggt tatgatggat tttgcttgat gttccacatt |
| | atgggttatt ctattttctt tgttcttgta agatgatgct tcagaagagt ttgttacttt |
| | ttaccgtatt tgtaatttac attttcactg aaaacaattg gcgagtaaaa aagtgtcctt |
| | gtcttcttct ttgttcggat tatatgaaca attgttccta gaagcctctc tacataaaaa |
| | gctgagactt tatctctcat ctctctttag acgtacaaaa aaatcagttt tttaagtttc |
| | actctaatgg cgtcaatttc gtcctttggc tgcttccctc aatccacagc gctcgccgga |
| | acttcctcca ccaccgtacg acgccgcacc atctctctgt ttcttcttct tcttcctttt |
| | tattcactga atc |
| 26 | attaagctct catttcggga agaattacta caaaagctac taatttgacc taattcatgc |
| | acaaatttga ttacaatgaa gaaataactt acaacgttga cgagcagaga aaccttgtag |
| | ccggtaattg tcggcgagag agcttctacc cttctggttg gattttttag ggttttagaa |
| | tttcattttc caacaaaaga taaacaaata aaaattggaa cttgtcgtta atacagccct |
| | ttaatgggtc aacgggtctt atgtctcttg aaaaagccca tgggccaaga caggtaaaat |
| | aacaatgtca ctttcgtaat tatcgcaaag tatatgcctt gttccatcag attccatttg |
| | cccaataaag cccgagtttc gagagttaat acctcattgg tgcttttggt tttggcaaag |
| | cgtgagtgag atcgggaatc aaacatcgcc tccgtctctc atttcaaacg ctatctccat |
| | ctccttcctc cgccgccgcc ATGGAATCTC CGAAGAATTC TCTGATCCCG AGCTTCCTCT |
| | ATTCATCATC TTCATCTCCG AGATCTTTCC TCCTCGACCA GGTGCTCAAT TCCAACTCCA |
| | ACGCTGCATT CGAGAAATCT CCTTCTCCGG CCCCGCGTTC CTCTCCTACG TCGATGATTT |
| | CTCGGAAGAA TTTCCTTATT GCATCTCCCA CCGAGCCAGG GAAGGGGATC GAGATGTATT |
| | CACCTGCCTT CTACGCTGCT TGTACCTTTG GTGGAATTCT CAGCTGTGGT CTTACTCACA |
| | TGACCGTGAC TCCTCTCGAT CTCGTCAAGT GCAATATGCA Ggtatgtaac ctttagatcc |
| | gttgtctttc gtttgttttc tgagctcatg tttgtggatc tgtgttcctg tgttgtttag |
| | gtagtgagat ctgtgttgct agatctgtga tttgattttc tttatcgctt tgttgttttc |
| | ctgactattg gttttgtgtt tgatttcaat atctgaagaa ttgtttgatc tctgataaac |
| | gcatcttcgt ctatccattt ccatgttata tatgaatcat tctatttcaa tatacgttaa |
| | tatggtctga tttctggttc ttctttcgaa atattgttac ttgacgtgtt atgtgttgaa |
| | tggttcactt ggtcttgcaa aactgatata tcttgttatc cagATTGATC CAGCGAAGTA |
| | CAAGAGCATC TCGTCTGGTT TTGGAATTTT GCTGAAAGAG CAAGGAGTCA AAGGCTTTTT |
| | CCGTGGATGG GTTCCTACTC TTTTGGGTTA CAGTGCTCAG GGTGCCTGCA AGTTTGGATT |
| | CTACGAGTAC TTTAAGAAGA CTTACTCTGA CCTTGCTGGA CCTGAGTACA CTGCCAAATA |
| | CAAGACTCTC ATCTACCTTG CTGGTTCTGC TTCTGCTGAG ATCATTGCCG ATATTGCACT |
| | TTGCCCATTT GAAGCTGTGA AGGTTCGTGT TCAGACACAG CCTGGATTTG CTAGGGGGAT |
| | GTCTGATGGA TTTCCCAAGT TTATCAAGTC CGAAGGATAC GGAGGgtgag tttttcaata |
| | ccaataacat tatctccctt gttactgcta gccttttggt ctgatttctg atttttttgc |
| | agCTTGTATA AGGGTCTTGC TCCACTCTGG GGACGTCAGA TTCCTTgtaa gttctggcct |
| | ctattttgca acctgttgca caatcttttt tttttttttt ttttgtttat tgatgaaaca |
| | tatgtagttc tttaaaagca aaaggtggtg atgatatcta tgaattttac agACACTATG |
| | ATGAAGTTTG CTTCCTTTGA GACCATTGTT GAGATGATTT ACAAGTACGC AATCCCCAAC |
| | CCAAAGAGTG AGTGCAGCAA AGGTCTGCAA CTCGGAGTGA GTTTTGCCGG AGGTTACGTT |
| | GCCGGAGTGT TCTGTGCCAT CGTTTCTCAT CCAGCAGACA ATCTAGTGTC ATTCCTCAAC |
| | AACGCTAAGG GAGCAACCGT TGGAGATgta agtcactatg tttgaataca atagcctaat |
| | gctagaatgg ctgtggtttg gtagttgtat acaagctatt gatttctgtt acggtagaaa |
| | taatatttaa tgtttgtaaa tgacatgttg cagGCGGTGA AGAAGATTGG TATGGTGGGA |
| | CTGTTCACAA GAGGGCTTCC TCTTAGAATT GTGATGATCG GGACGTTGAC TGGAGCACAG |
| | TGGGGATTAT ACGATGCCTT CAAAGTGTTT GTTGGCCTgt aagttcctct ctctcttcac |
| | ttactttcgt accttaattg taccttcaaa atgcaaaact ctcaattctt ttgatttggt |
| | attcagGCCA ACCACTGGTG GTGTTGCTCC AGCTCCTGCC ATCGCAGCTA CTGAAGCCAA |
| | AGCCTAAaca atgacgaaaa aggttattag gagttcgatg gggtaggatt tttgtttgga |
| | aaaataagag aaaccatacg gtgatgagga agagtgagta agctcaattt cttcctgatt |
| | tgaactttat catttttgtt ttttttgaaa tttgtgttcc tgaattcagg atagtgctct |
| | ctctctcttt acatactctc ttcctattgt ttcttgtcct ttttttcttt gtgtgatgta |
| | atcttaaaag atgagaggga cacactccaa gatagagaga gtgggcatac acccactcac |
| | tactttttat tcagtttcag ttgaaattct cttttggttg ctctatctat tattttactt |
| | ttttgtttta gagattatat aaaatctcgt tttaaaacat caaatcatag atagatcttg |
| | aatactaatc atatgtatac gtttaaccgc taagcgctaa cataaggaaa atattatgta |
| | ggcaaatgat taataaacat atgataa |
| 27 | aatgattttg acctttttaa ataatatatt caaatgtgtt tcaaacacga atcaaactat |
| | accaaaaaaa aaaaaaaagt tggataaaaa ataaaacctg actacacctc aactttggat |
| | caaaatctat gaatatattt tcaaaattat cttagtcaaa ttttaaatta attaattatt |
| | tatataaaat ttaataatta tcataacctt ggattaaatt tatctacagt caaaaattaa |
| | ttttaaatca attaattaat agcattatta caatccctaa ttgtacggga cgaataaaaa |
| | agtagaaaac tcaagttcct ttctttacca tacagctttt tcgattggag ttgaataagt |
| | cttcatctga cacgtgtaac cctggcacat gccgtccact aaaacacgtg cgagatctgt |
| | ataaatcaaa cctacgcgtt tcatctctct tttcaaaact caccgacgcg atccgatctc |
| | atctctctca tttcgaaacc ATGGTTGAGC CGGCGAATAC TGTTGGTCTT CCGGTGAACC |
| | CGACTCCGTT GCTGAAAGAT GAGCTCGATA TCGTGATTCC GACTATCAGA AACCTCGATT |
| | TCCTCGAGAT GTGGAGGCCT TTTCTTCAGC CTTACCATCT GATCATCGTC CAGGACGGAG |
| | ATCCATCGAA GAAGATCCAT GTCCCTGAAG GTTACGACTA CGAGCTCTAC AACAGGAACG |
| | ACATTAACCG AATCCTCGGA CCTAAGGCTT CTTGTATCTC GTTTAAGGAT TCTGCTTGTC |
| | GATGCTTTGG GTACATGGTG TCTAAGAAGA AGTATATCTT CACCATTGAT GACGATTGCT |
| | TCgtaagtta cttgaatttt gagttttgta ttcgttttta tgcttgattt gagagttttg |
| | tcaattttgg ttctagatct gtttttttga gcttatttgt ttgtgtttgt gtggattttt |
| | caagttcatt gcttgaattt cgtagatttg gtgagagatc aattatacga ttcactaaat |
| | ttgacggatc ttaggtttgt gagataatcc ttggttcgat tagctaggca attcaatgtt |
| | ttgtaccaga tccatagatc tgcttgttga gtctgaatat gttttcactt ttgtgtaatt |
| | agccatgatc tctaatgttt acttgtagat tttctgtgag ctgatgtctc ttttgttgac |
| | gacattgttg ttgagctgat atctctgagt cattatagct acctttacga tatggttgca |
| | cgtccttgtt catcactttt ttcttttgtt ttaccttttt gagatttgtg gggcatatcc |
| | aaggatgagt ctcgatgacg cttgtgttta gtttataatt ttctgagttt tttttggagg |
| | aactctttga tcaatggctt gatctggatt ttaaccgctt tttaattcat gtatttcttt |
| | gatgtgtaca tgtagGTTGC CAAGGATCCA TCAGGCAAAG CAGTGAACGC TCTTGAGCAA |
| | CACATCAAGA ACCTTCTCTG CCCATCGTCT CCCTTTTTCT TCAACACCTT GTATGATCCT |
| | TACCGTGAAG GTGCTGATTT CGTCCGTGGA TACCCTTTCA GTCTCCGTGA AGGTGTTTCC |
| | ACTGCTGTTT CCCATGGTCT TTGGCTCAAC ATCCCTGACT ACGATGCCCC GACCCAACTC |
| | GTGAAGCCTA AGGAGAGGAA CACCAGgtga caataattat catcataaca tgtttatgtg |
| | tttttttgtc aggatattca aatgtcagtt tttgctaaac gtttgatatg tcagGTATGT |
| | GGATGCTGTC ATGACCATCC CAAAGGGAAC ACTTTTCCCA ATGTGTGGTA TGAACTTGGC |
| | TTTTGACCGT GATTTGATTG GCCCGGCTAT GTACTTTGGT CTCATGGGTG ATGGTCAGCC |
| | TATTGGTCGT TACGACGATA TGTGGGCTGG TTGGTGCATC AAGgtaattt cttcttattc |
| | ccttgtaaga ctcataattg agtatagcta aatatgaagc acatgctctg tactaagcga |
| | tacctccatt tggggttgaa tcttttatag GTGATCTGTG ACCACTTGAG CTTGGGAGTG |
| | AAGACCGGTT TACCGTATAT CTACCACAGC AAAGCGAGCA ACCCTTTTGT TAACCTGAAG |
| | AAGGAATACA AGGGAATCTT CTGGCAGGAG GAGATCATTC CGTTCTTCCA GAACGCAAAG |
| | CTATCGAAAG AAGCAGTAAC TGTTCAGCAA TGCTACATTG AGCTCTCAAA GATGGTCAAG |
| | GAGAAGTTGA GCTCCTTAGA CCCGTACTTT GACAAGCTTG CAGATGCCAT GGTTACATGG |
| | ATTGAAGCTT GGGATGAGCT TAACCCACCA GCAGCCAGTG GCAAAGCTTG Agagcagtat |
| | gagccaaaaa gaaaaagcca ccaaagtttt ggttattttt agctcaaatt atcgttactt |
| | ttaaatttct gattttacga acctttcttg ctttttttac acatttgagt agttttcatc |
| | atcagtactt tctcattgtc cggttatggt ttttgcattt ggtttaaata tcaccggttt |
| | atttataaac agtggtggat tagtagtact attttctgag tttttttctt tgtttcatta |
| | ataaaaaggc cttttcatag gtgtttgcaa ttagtttttt tcccccatta atcatcgatt |
| | atcataggta tgttatggct ttaaatggta taaggaaatt gcttatagac caaaaaaaag |
| | ttgaattgct attgagagag cttttacaaa agaaagagca ttgttcaata agcttttcac |
| | atttggtcga tattttgatc aacctatcat aggtatctca attaataaac cggaatgtta |
| | atatgttttg c |
| 28 | ttctttaatt tcttcgccaa gaagagcacg aaatgtttgc caaacgcata tgcaacaacc |
| | ccacgttaca tatttctatt tgtagctata gagcaagcta tattgttaaa aactaaaaag |
| | aaaatcttta ctataacata tagatagagg attcgagata tcttgaaaga ctcaacttaa |
| | taaataaagt cgaaaagaaa acacggaggc gagaggacca cacactcgca cagaaagagt |
| | ctcatatcct ctataacaaa ttgataaact aaactaaaac gacacgtgat gtcttgatca |
| | gccaataaaa agctaccgac ataaggcaaa aatgatcgta ccattaaacg taatccacgt |
| | ggtttcagat tacacgtggc accacacaag tatctccatt tggcctataa atataaaccc |
| | ttaagcccac atatcttctc aatccatcac aaacaaaaca cacatcaaaa acgattttac |
| | aagaaaaaaa tatctgaaaa ATGTCAGAGA CCAACAAGAA TGCCTTCCAA GCCGGTCAGG |
| | CCGCTGGCAA AGCTGAGgta ctctttctct cttagaacag agtactgata gattgttcaa |
| | gttataactc tttgaaaaca gttgaaactt gatcactcct agaacttcca ttttcttgtt |
| | taatttagtt tgtcgtaatt atgtaattga ttttgtgttg accatggttg ttatatagGA |
| | GAAGAGCAAT GTTCTGCTGG ACAAGGCCAA GGATGCTGCT GCTGCAGCTG GAGCTTCCGC |
| | GCAACAGgta aacgatctat acacacatta tgacatttat gtaaagaatg aaaagtcttc |
| | ttagagcata catttacgca gatttctgat attttcatat ggtttgatgt aaatgttata |
| | gGCGGGAAAG AGTATATCGG ATGCGGCAGT GGGAGGTGTT AACTTCGTGA AGGACAAGAC |
| | CGGCCTGAAC AAGTAGcgat ccgagtcaac tttgggagtt ataatttccc ttttctaatt |
| | aattgttggg attttcaaat aaaatttggg agtcataatt gattctcgta ctcatcgtac |
| | ttgttgttgt ttttagtgtt gtaatgtttt aatgtttctt ctccctttag atgtactacg |
| | tttggaactt taagtttaat caacaaaatc tagtttaagt tctaagaact ttgttttacc |
| | atcctctttt ttattgcact taatgcttat agacttttat gtccatccat ttctcaattc |
| | ggctacgttg aattataagg gtcacataag caaaaaaata tcttaaaaag tcataacatt |
| | aaggcaaaga tagattctta aaagtactca aattgagatc acgaaaataa caagttagaa |
| | gttagaactt ccgtaggata tttataagaa caaaagatta ataaatgaag gcaatgattc |
| | tggattcctt gcaagttagg aagttcgaaa tcgttg |
| 29 | cgttattatt actacttcgc ttttagtgtg attcgtttca ttctcgtttt tttatattcc |
| | tcgatctgtt tgctcatttg ttgagatcta ttcgctatgt gagttcattt gactcagatc |
| | tggatatttc gtgttgttcg atttatagat ctggtttctg gatctgttta cgatctatcg |
| | tcatctttcc tttgaaaatg attggtgttt ctgtgttcgt attcgtttag atctaaagtt |
| | tttgatcgat gaatgtcgca tgtgttttta tctgaaagtt ttcgattaca gtatcaagtg |
| | gtggtagtag tagtagtaga ctcaaaaagc tgcacaaact ttttatacac gtgaattgtg |
| | attgctttac ggttttcttg gagtttgtta attaaatcat ttaatattaa gaagtttatg |
| | aattaagaga acgttatttt atactatgat tttgattttg atttggtttg tgtgttttaa |
| | tgcagtaaaa gaaaatcaaa ATGGCTTCAC ACATTGTTGG ATACCCACGT ATGGGCCCTA |
| | AGAGAGAGCT CAAGTTTGCA TTGGAATCTT TCTGGGATGG TAAGAGCACT GCTGAGGATC |
| | TTCAGAAGGT GTCTGCTGAT CTCAGGTCAT CCATCTGGAA ACAGATGTCT GCCGCTGGGA |
| | CTAAGTTCAT CCCTAGCAAC ACCTTTGCTC ACTACGACCA GGTTCTTGAC ACCACCGCCA |
| | TGCTCGGTGC TGTTCCACCT AGGTATGGAT ACACTGGTGG TGAGATCGGC CTTGATGTTT |
| | ACTTCTCCAT GGCTAGAGGA AATGCCTCTG TGCCTGCCAT GGAAATGACC AAGTGGTTCG |
| | ACACCAACTA gtgagtcttc attgatctct tgtgttcttt ttgttgacat tggtcttttt |
| | gagttgtgga ctaatttgat tatgcttttg ttgatgcagC CATTACATCG TCCCTGAGTT |
| | GGGCCCTGAG GTTAACTTCT CTTACGCATC CCACAAGGCG GTGAATGAGT ACAAGGAGGC |
| | CAAGGCTgta cgtatcattc tttactaata tccgtttctt aggaaattac tgtttgctcg |
| | tctaattaac tattagagat cataggcttt agtttgagga tatagtgttt aagcttagat |
| | tcattgagtg gtgtttcact gaggatgcta atatgctagg aaggtctcgg atgcattgaa |
| | tataaaaacc gttagaaaag tcatctggca ctggttgtct aaagtagttt ttttttctac |
| | gaagttctga tctggtttac ttgatgttta tgcagCTTGG TGTTGACACC GTCCCTGTAC |
| | TTGTTGGCCC AGTCTCTTAC TTGCTGCTTT CCAAGGCTGC CAAGGGTGTT GACAAGTCAT |
| | TCGAACTTCT TTCTCTTCTC CCTAAGATTC TCCCGATCTA CAAgtaagaa atcactttat |
| | tgtttttctt tattatgcca tccgtatcct tgatgttatc aatgatcctc tgacatacca |
| | ctgatataat gactttgatt tgtgtacagG GAAGTGATTA CCGAGCTTAA GGCTGCTGGT |
| | GCCACCTGGA TTCAGCTTGA CGAGCCTGTC CTTGTTATGG ATCTTGAGGG TCAGAAACTC |
| | CAGGCCTTTA CTGGTGCCTA TGCTGAACTT GAATCAACTC TTTCTGGTTT GAATGTTCTT |
| | GTCGAGACCT ACTTCGCTGA TATCCCTGCT GAGGCATACA AGACCCTAAC CTCATTGAAG |
| | GGTGTGACTG CCTTTGGATT TGATTTGGTT CGTGGCACCA AGACCCTTGA TTTGGTCAAG |
| | GCAGGTTTCC CTGAGGGAAA GTACCTCTTT GCTGGTGTTG TTGATGGAAG GAACATCTGG |
| | GCCAACGACT TTGCTGCGTC CCTAAGCACC TTGCAGGCAC TTGAAGGCAT TGTTGGTAAA |
| | Ggtaattgtt cttccaaaat catctgcctt ttacctgaca ttactaggga attattgaaa |
| | aacaactgta tgaaatgttg atctgttgtc tttttgatgc agACAAGCTT GTGGTCTCAA |
| | CCTCCTGCTC TCTTCTCCAC ACCGCTGTTG ATCTTATCAA TGAGACTAAG CTTGATGATG |
| | AAATCAAGTC ATGGTTGGCG TTTGCTGCCC AGAAGGTCGT TGAAGTGAAC GCTTTGGCCA |
| | AGGCTTTGGC TGGTCAGAAG GACGAGgtat tttacccaca tgctccccta gtagtggacc |
| | cttgaattat ctgtagtgta attgatccag aaaaatctag aactcaatat tttttttctt |
| | tcagGCTCTT TTCTCTGCCA ATGCTGCGGC TTTGGCTTCA AGGAGATCTT CCCCAAGAGT |
| | CACCAACGAG GGTGTCCAGA AGGCTgtaag tttgatttca aactgatgca ctgtgctcac |
| | ccaatggttt attttcctaa tcttgtattg attgagatag tttctcattc ttgttatctc |
| | agGCTGCTGC TTTGAAGGGA TCTGACCACC GTCGTGCAAC CAATGTTAGT GCTAGGCTAG |
| | ATGCTCAGCA GAAGAAGCTC AATCTCCCAA TCCTACCAAC CACAACCATT GGATCCTTCC |
| | CACAGACTGT AGAGCTCAGG AGAGTTCGTC GTGAGTACAA GGCCAAAAAg ttagtctcct |
| | aaatttaatc cttgggctta tgcgtcacac attttcttaa attgttgtga tgctaatggt |
| | ttctttaatc tctcttttac tagGGTCTCA GAGGAGGACT ACGTTAAAGC CATCAAGGAA |
| | GAGATCAAGA AAGTTGTTGA CCTCCAAGAG GAACTTGACA TCGATGTTCT TGTCCACGGA |
| | GAGCCAGAGg tgaatttttt ttattattct atgtttttgc ctgatatttc tagtaatcct |
| | tggtactgtt tctgatgaga catgttttca caattttgta gAGAAACGAC ATGGTTGAGT |
| | ACTTTGGTGA GCAGTTGTCT GGTTTTGCCT TCACTGCAAA CGGATGGGTC CAATCTTATG |
| | GATCTCGCTG TGTGAAGCCA CCAGTTATCT ATGGTGATGT GAGCCGTCCC AAGGCAATGA |
| | CCGTCTTCTG GTCCGCAATG GCTCAGAGCA TGACCTCTCG CCCAATGAAG GGTATGCTTA |
| | CTGGTCCCGT CACCATTCTC AACTGGTCCT TTGTCAGGAA CGACCAGCCC AGgtacataa |
| | tgttactata atctaaaaac aaacataaac accaaataaa gaacaaaaca ctaagacaat |
| | cttggaatca ttgtagGCAC GAAACCTGTT ACCAGATCGC TTTGGCCATC AAGGACGAAG |
| | TCGAGGATCT TGAGAAAGGT GGAATCGGTG TCATTCAGAT TGATGAGGCT GCACTTAGAG |
| | AAGGACTACC ACTCAGGAAA TCCGAGCATG CTTTCTACTT GGACTGGGCC GTCCACTCCT |
| | TCAGAATCAC CAACTGTGGA GTCCAAGACA GCACCCAGgt ttgcttaaat aaaaactaca |
| | cataacgagt ctcatgtagt gtaatgcttt ctcagttgct cataacttat gtgtttctgg |
| | tgtttttttt ttgcagATCC ACACTCACAT GTGCTACTCC CACTTCAATG ACATCATACA |
| | CTCCATCATC GACATGGATG CTGATGTCAT CACCATTGAG AACTCCAGGT CTGATGAGAA |
| | GCTTCTTTCC GTGTTCCGTG AAGGAGTGAA GTACGGTGCT GGAATCGGTC CAGGAGTCTA |
| | CGACATCCAC TCTCCAAGAA TACCATCTTC TGAGGAAATC GCAGACAGGG TCAACAAGAT |
| | GCTTGCTGTC CTAGAGCAGA ACATCCTTTG GGTTAACCCT GACTGTGGTC TCAAGACCCG |
| | TAAGTACACC GAGGTCAAGC CTGCACTCAA GAACATGGTT GATGCGGCTA AGCTCATCCG |
| | CTCCCAGCTC GCCAGTGCCA AGTGAagaaa agcttgattt gaacaaggaa acgttttttt |
| | ttctctaaaa tggttgtgtt ttatttggtt taataacttt cttaaaaata tttttagtcg |
| | aaggtagatt tgatgcatat ggtttctttc ttgttgagag agagaaaggc tatagcatcc |
| | tttggatttg atgcaatgtt tgtgattttc tttttgtctc caatatattt ctctgatgga |
| | atgtcttttt tctaaagtat cttgaaaagg aataagagga ttgattctta tacaaatact |
| | tttgtttgcg ttgtcctaaa ctcactactt ttttttatcc gacgcaatca gtgctttgta |
| | gcctgttctt gaagtaggcc cctttgtatg tctctatctg gctcctgtat cagattgttg |
| | tttcccttag atttctttat ttcgttggca aaaagaaaat ctgaattgcc ccacaaagag |
| | cgtggtggct gatgttaggt tgcagtctca tggtccacca cttta |
| 30 | ttttgcagaa acattacatt acagatggag aacgccaaaa atcgattctt ttttttaatt |
| | ttcttttttg acaaatcgca ttctgcacac attccttttt tttttaattt tctccactac |
| | accactaatc ttgccgtgat aggtgcatgt gtatgtgttt aagacatatc tcttttgttc |
| | cggttggatt agtttatgta ataaccaaca actatactta atacattttg tccacttttg |
| | aattttctgt ttcttatttt gtttactgta aaaaagaatg aaaatcattg agatattaaa |
| | actaactaat cactaaggcc catttagtag acccaataag gcccatatgc tatttttttt |
| | ctccagaatt tgacctttat gtatttgacc gagtggaaaa gtaatacagt tcttttcttc |
| | tctcctcctc tttcttcttc atgattggaa ttttagggct tttgaaagca cgaacgcgtg |
| | aagctctaat cgagaaaaaa ATGGAGGTTT TGGATAGGAG AGACGATGAG ATCAGGGACT |
| | CGGGAAACAT GGACAGCATC AAGTCACACT ATGTTACCGA CTCTGTTTCC GAGGAACGCC |
| | GCTCTCGTGA GCTCAAGGAT GGAGACCATC CTTTACGGgt ttgtccttta tccttagtat |
| | cgattcattt gcaatttgaa tctgatctta gctgaaaatt tgattcccgt tcgtcaaaga |
| | tttctgaact ggtgatatga cggtttatag ctagagtagt ggaagattcg gattctaaat |
| | ctttgtttgt tggagttttt gttttcaaat taggttttgc gaatttgttt agatgtatgt |
| | gagctcaaat gttataggat tttcgtattg gtggtattga ttgtagctag aacaaggcag |
| | attgatttag aggaactgat ttcattgtta agagtaagta ctggctcagt gactctagga |
| | tttttggtaa tgatgcagTA CAAGTTTTCG ATATGGTACA CTCGTCGCAC ACCAGGGGTT |
| | CGGAACCAGT CTTATGAAGA TAACATCAAG AAGATGGTAG AATTCAGCAC Ggtaagtcta |
| | aatatactac tggaagttca ttgttgaagc tgtttgcgat actatcttgt tcgtttctga |
| | gttatggctt ttataaacta gGTTGAAGGA TTTTGGGCCT GCTACTGTCA CCTTGCTCGT |
| | TCTTCTCTCT TGCCTAGTCC AACAGATCTT CATTTCTTTA AGGATGGGAT TCGTCCATTG |
| | TGGGAGgtac gtattcccct gtgttgattt ttcgtattgt gtttttatct ggatcatcga |
| | tatagaggga accttttata caacaaaagt ttctcaagag ttgtatcttc ttcaataaac |
| | caactaaact agctaaattc atcaccttta gGATGGTGCC AACTGCAATG GAGGAAAGTG |
| | GATCATACGT TTCTCAAAAG TTGTATCTGC TCGCTTCTGG GAGGATCTGg tgagttttat |
| | tttcttgtgg gcactactat tggagtattg acacctttct actttattca aaagaaaccc |
| | ttttgtcaat gttatttata atccatttta catacttagg gtctgagaat catgttaaat |
| | actcttccgt ttatttgttt tcttcagCTT CTTGCGTTGG TAGGCGACCA GCTTGATGAT |
| | GCTGATAACA TATGTGGGGC AGTACTGAGT GTCCGTTTCA ACGAGGACAT CATTAGTGTA |
| | TGGAATCGCA ATGCTTCTGA CCATCAGgtg agaaaactgt tcacaagaag aactgtctct |
| | ctccctctcc ttttgattgg tacttacaca gtgcaatgtt ttccttaaac agGCAGTGAT |
| | GGGTTTGAGA GACTCAATCA AGCGGCATTT GAAGTTGCCT CATGCATATG TCATGGAATA |
| | CAAGCCACAC GATGCTTCTC TCCGCGACAA CTCTTCCTAC AGAAACACAT GGCTGAGAGG |
| | ATAGgcccaa agtcgatgat tgtatcatgt aatgtggaga agatttggga agctcatctg |
| | caacctggga agatatctgg attgaaccct gtatccaata ccatactgta ccggaggctt |
| | acaatatcag aaaaaacaaa atccgggcta cttctgtgtc agtatgtgtt catttcgttt |
| | ttcttttaca gtacatcttg ttaacttcaa tggtttgact cttgatcaaa actataagga |
| | tgtattttca atgaaaactg gaaattacgt tctggtttac attataactc atgtcttaaa |
| | aagtaacagg atgtcaatat acaatgtcac ttcgtacgat gatctctaat gtacatctac |
| | tgatgaaaaa ctgagtgtgg ctctgtccgt tgatctcaaa agctatagtt tagcatccgc |
| | agatgattga agtccgatga tacctggttc aacatcaaag cctcgagtga attacttcac |
| | acaatggaaa ctagaaaata agag |
| 31 | MALKSKLVSL LFLIATLSST FAASFSDSDS DSDLLNELVS LRSTSESGVI HLDDHGISKF |
| | LTSASTPRPY SLLVFFDATQ LHSKNELRLQ ELRREFGIVS ASFLANNNGS EGTKLFFCEI |
| | EFSKSQSSFQ LFGVNALPHI RLVSPSISNL RDESGQMDQS DYSRLAESMA EFVEQRTKLK |
| | VGPIQRPPLL SKPQIGIIVA LIVIATPFII KRVLKGETIL HDTRLWLSGA IFIYFFSVAG |
| | TMHNIIRKMP MFLQDRNDPN KLVFFYQGSG MQLGAEGFAV GFLYTVVGLL LAFVTNVLVR |
| | VKNITAQRLI MLLALFISFW AVKKVVYLDN WKTGYGIHPY WPSSWR* |
| 32 | MTKTMMIFAA AMTVMALLLV PTIEAQTECV SKLVPCFNDL NTTTTPVKEC CDSIKEAVEK |
| | ELTCLCTIYT SPGLLAQFNV TTEKALGLSR RCNVTTDLSA CTAKGAPSPK ASLPPPAPAG |
| | NTKKDAGAGN KLAGYGVTTV ILSLISSIFF * |
| 33 | MAAITEFLPK EYGYVVLVLV FYCFLNLWMG AQVGRARKRY NVPYPTLYAI ESENKDAKLF |
| | NCVQRGHQNS LEMMPMYFIL MILGGMKHPC ICTGLGLLYN VSRFFYFKGY ATGDPMKRLT |
| | IGKYGFLGLL GLMICTISFG VTLILA* |
| 34 | MSLLADLVNL DISDNSEKII AEYIWVGGSG MDMRSKARTL PGPVTDPSKL PKWNYDGSST |
| | GQAPGQDSEV ILYPQAIFKD PFRRGNNILV MCDAYTPAGE PIPTNKRHAA AEIFANPDVI |
| | AEVPWYGIEQ EYTLLQKDVN WPLGWPIGGF PGPQGPYYCS IGADKSFGRD IVDAHYKASL |
| | YAGINISGIN GEVMPGQWEF QVGPSVGISA ADEIWIARYI LERITEIAGV VVSFDPKPIP |
| | GDWNGAGAHT NYSTKSMREE GGYEIIKKAI EKLGLRHKEH ISAYGEGNER RLTGHHETAD |
| | INTFLWGVAN RGASIRVGRD TEKEGKGYFE DRRPASNMDP YVVTSMIAET TLLWNP* |
| 35 | MYQKFQISGK IVKTLGLKMK VLIAVSFGSL LFILSYSNNF NNKLLDATTK VDIKETEKPV |
| | DKLIGGLLTA DFDEGSCLSR YHKYFLYRKP SPYKPSEYLV SKLRSYEMLH KRCGPDTEYY |
| | KEAIEKLSRD DASESNGECR YIVWVAGYGL GNRLLTLASV FLYALLTERI ILVDNRKDVS |
| | DLLCEPFPGT SWLLPLDFPM LNYTYAWGYN KEYPRCYGTM SEKHSINSTS IPPHLYMHNL |
| | HDSRDSDKLF VCQKDQSLID KVPWLIVQAN VYFVPSLWFN PTFQTELVKL FPQKETVFHH |
| | LARYLFHPTN EVWDMVTDYY HAHLSKADER LGIQIRVFGK PDGRFKHVID QVISCTQREK |
| | LLPEFATPEE SKVNISKTPK LKSVLVASLY PEFSGNLTNM FSKRPSSTGE IVEVYQPSGE |
| | RVQQTDKKSH DQKALAEMYL LSLTDNIVTS ARSTFGYVSY SLGGLKPWLL YQPTNFTTPN |
| | PPCVRSKSME PCYLTPPSHG CEADWGTNSG KILPFVRHCE DLIYGGLKLY DEF* |
| 36 | MRTVVHDLAV VLLVIFYDYY MLFILDRLLE ANYGGKWEKI LGNHVDIFKN YPLIGQLFVQ |
| | DMYNSIMDFP SFFIFQALLE YERHKVSEGE LQIPLPLELE PMNIDNQASG SGRARRDAAS |
| | RAMQGWHSQR LNGNGEVSDP AIKDKNLVLH QKREKQIGTT PGLLKRKRAA EHGAKNAIHV |
| | SKSMLDVTVV DVGPPADWVK INVQRTQDCF EVYALVPGLV REEVRVQSDP AGRLVISGEP |
| | ENPMNPWGAT PFKKVVSLPT RIDPHHTSAV VTLNGQLFVR VPLEQLE* |
| 37 | MSWQSYVDDH LMCDVEGNHL TAAAILGQDG SVWAQSAKFP QLKPQEIDGI KKDFEEPGFL |
| | APTGLFLGGE KYMVIQGEQG AVIRGKKGPG GGVIKKTNQA LVFGFYDEPM TGGQCNLVVE |
| | RLGDYLIESE L* |
| 38 | MYVVKRDGRQ ETVHFDKITA RLKKLSYGLS SDHCDPVLVA QKVCAGVYKG VTTSQLDELA |
| | AETAAAMTCN HPDYASLAAR IAVSNLHKNT KKSFSETIKD MFYEVNDRSG LKSPLIADDV |
| | FEIIMQNAAR LDSEIIYDRD FEYDYFGFKT LERSYLLKVQ GTVVERPQHM LMRVAVGIHK |
| | DDIDSVIQTY HLMSQRWFTH ASPTLFNAGT PRPQLSSCFL VCMKDDSIEG IYETLKECAV |
| | ISKSAGGIGV SVHNIRATGS YIRGTNGTSN GIVPMLRVFN DTARYVDQGG GKRKGAFAVY |
| | LEPWHADVYE FLELRKNHGK EEHRARDLFY ALWLPDLFME RVQNNGQWSL FCPNEAPGLA |
| | DCWGAEFETL YTKYEREGKA KKVVQAQQLW YEILTSQVET GTPYMLFKDS CNRKSNQQNL |
| | GTIKSSNLCT EIIEYTSPTE TAVCNLASIA LPRFVREKGV PLDSHPPKLA GSLDSKNRYF |
| | DFEKLAEVTA TVTVNLNKII DVNYYPVETA KTSNMRHRPI GIGVQGLADA FILLGMPFDS |
| | PEAQQLNKDI FETIYYHALK ASTELAARLG PYETYAGSPV SKGILQPDMW NVIPSDRWDW |
| | AVLRDMISKN GVRNSLLVAP MPTASTSQIL GNNECFEPYT SNIYSRRVLS GEFVVVNKHL |
| | LHDLTDMGLW TPTLKNKLIN ENGSIVNVAE IPDDLKAIYR TVWEIKQRTV VDMAADRGCY |
| | IDQSQSLNIH MDKPNFAKLT SLHFYTWKKG LKTGMYYLRS RAAADAIKFT VDTAMLKEKP |
| | SVAEGDKEVE EEDNETKLAQ MVCSLTNPEE CLACGS* |
| 39 | MAYASRFLSR SKQLQGGLVI LQQQHAIPVR AFAKEAARPT FKGDEMLKGV FFDIKNKFQA |
| | AVDILRKEKI TLDPEDPAAV KQYANVMKTI RQKADMFSES QRIKHDIDTE TQDIPDARAY |
| | LLKLQEIRTR RGLTDELGAE AMMFEALEKV EKDIKKPLLR SDKKGMDLLV AEFEKGNKKL |
| | GIRKEDLPKY EENLELSMAK AQLDELKSDA VEAMESQKKK EEFQDEEMPD VKSLDIRNFI |
| | * |
| 40 | MHGYEDDLDE EAGYDDYYSG DEDEYEDEEE EDEEPPKEEL EFLESRQKLK ESIRKKMGNG |
| | SANAQSSQER RRKLPYNDFG SFFGPSRPVI SSRVIQESKS LLENELRKMS NSSQTMFLLM |
| | ELFFGVQKKR PVPTNGSGSK NVSQEKRPKV VNEVRRKVET LKDTRDYSFL FSDDAELPVP |
| | KKESLSRSGS FPNSAYHFHE DNLYRFFADV QEARSAQLSS RPKQSSGING RTAHSPHREE |
| | KRPVSANGHS RPSSSGSQMN HSRPSSSGSK MNHSRPATSG SQMPNSRPAS SGSQMQSRAV |
| | SGSGRPASSG SQMQNSRPQN SRPASAGSQM QQRPASSGSQ RPASSGSQRP ASSGSQRPGS |
| | STNRQAPMRP PGSGSTMNGQ SANRNGQLNS RSDSRRSAPA KVPVDHRKQM SSSNGVGPGR |
| | SATNARPLPS KSSLERKPSI SAGKSSLQSP QRPSSSRPMS SDPRQRVVEQ RKVSRDMATP |
| | RMIPKQSAPT SKHQMMSKPA LKRPPSRDID HERRLLKKKK PARSEDQEAF DMLRQLLPPK |
| | RFSRYDDDDI NMEAGFEDIQ KEERRSARIA REEDERELKL LEEEERRERL KKNRKLSR* |
| 41 | MDPNQRIARI SAHLNPPNLH NQIADGSGLN RVACRAKGGS PGFKVAILGA AGGIGQPLAM |
| | LMKMNPLVSV LHLYDVANAP GVTADISHMD TSAVVRGFLG QPQLEEALTG MDLVIIPAGV |
| | PRKPGMTRDD LFNINAGIVR TLSEAIAKCC PKAIVNIISN PVNSTVPIAA EVFKKAGTFD |
| | PKKLMGVTML DVVRANTFVA EVMSLDPREV EVPVVGGHAG VTILPLLSQV KPPCSFTQKE |
| | IEYLTDRIQN GGTEVVEAKA GAGSATLSMA YAAVEFADAC LRGLRGDANI VECAYVASHV |
| | TELPFFASKV RLGRCGIDEV YGLGPLNEYE RMGLEKAKKE LSVSIHKGVT FAKK* |
| 42 | MAQVQAPSSH SPPPPAVVND GAATASATPG IGVGGGGDGV THGALCSLYV GDLDFNVTDS |
| | QLYDYFTEVC QVVSVRVCRD AATNTSLGYG YVNYSNTDDA EKAMQKLNYS YLNGKMIRIT |
| | YSSRDSSARR SGVGNLFVKN LDRSVDNKTL HEAFSGCGTI VSCKVATDHM GQSRGYGFVQ |
| | FDTEDSAKNA TEKLNGKVLN DKQIFVGPFL RKEERESAAD KMKFTNVYVK NLSEATTDDE |
| | LKTTPGQYGS ISSAVVMRDG DGKSRCFGFV NFENPEDAAR AVEALNGKKF DDKEWYVGKA |
| | QKKSERELEL SRRYEQGSSD GGNKFDGLNL YVKNLDDTVT DEKLRELFAE FGTITSCKVM |
| | RDPSGTSKGS GFVAFSAASE ASRVLNEMNG KMVGGKPLYV ALAQRKEERR AKLQAQFSQM |
| | RPAFIPGVGP RMPIFTGGAP GLGQQIFYGQ GPPPIIPHQP GFGYQPQLVP GMRPAFFGGP |
| | MMQPGQQGPR PGGRRSGDGP MRHQHQQPMP YMQPQMMPRG RGYRYPSGGR NMPDGPMPGG |
| | MVPVAYDMNV MPYSQPMSAG QLATSLANAT PAQQRTLLGE SLYPLVDQIE SEHAAKVTGM |
| | LLEMDQTEVL HLLESPEALN AKVSEALDVL RNVNQPSSQG SEGNKSGSPS DLLASLSIND |
| | HL* |
| 43 | MAENYDRASE LKAFDEMKIG VKGLVDAGVT KVPRIFHNPH VNVANPKPTS TVVMIPTIDL |
| | GGVFESTVVR ESVVAKVKDA MEKFGFFQAI NHGVPLDVME KMINGIRRFH DQDPEVRKMF |
| | YTRDKTKKLK YHSNADLYES PAASWRDTLS CVMAPDVPKA QDLPEVCGEI MLEYSKEVMK |
| | LAELMFEILS EALGLSPNHL KEMDCAKGLW MLCHCFPPCP EPNRTFGGAQ HTDRSFLTIL |
| | LNDNNGGLQV LYDGYWIDVP PNPEALIFNV GDFLQLISND KFVSMEHRIL ANGGEEPRIS |
| | VACFFVHTFT SPSSRVYGPI KELLSELNPP KYRDTTSESS NHYVARKPNG NSSLDHLRI* |
| 44 | MYKLDRKLGK GGFGQVYVGR KMGTSTSNAR FGPGALEVAL KFEHRTSKGC NYGPPYEWQV |
| | YNALGGSHGV PRVHFKGRQG DFYVMVMDIL GPSLWDVWNS TTQAMSTEMV ACIAIEAISI |
| | LEKMHSRGYV HGDVKPENFL LGPPGTPEEK KLFLVDLGLA SKWRDTATGL HVEYDQRPDV |
| | FRGTVRYASV HAHLGRTCSR RDDLESLAYT LVFLLRGRLP WQGYQVGDTK NKGFLVCKKK |
| | MATSPETLCC FCPQPFRQFV EYVVNLKFDE EPDYAKYVSL FDGIVGPNPD IRPINTEGAQ |
| | KVIW* |
| 45 | MAQRLEAKGG KGGNQWDDGA DHENVTKIHV RGGLEGIQFI KFEYVKAGQT VVGPIHGVSG |
| | KGFTQTFEIN HLNGEHVVSV KGCYDNISGV IQALQFETNQ RSSEVMGYDD TGTKFTLEIS |
| | GNKITGFHGS ADANLKSLGA YFTPPPPIKQ EYQGGTGGSP WDHGIYTGIR KVYVTFSPVS |
| | ISHIKVDYDK DGKVETRQDG DMLGENRVQG QPNEFVVDYP YEYITSIEVT CDKVSGNTNR |
| | VRSLSFKTSK DRTSPTYGRK SERTFVFESK GRALVGLHGR CCWAIDALGA HFGAPPIPPP |
| | PPTEKLQGSG GDGGESWDDG AFDGVRKIYV GQGENGIASV KFVYDKNNQL VLGEEHGKHT |
| | LLGYEEFELD YPSEYITAVE GYYDKVFGSE SSVIVMLKFK TNKRTSPPYG MDAGVSFILG |
| | KEGHKVVGFH GKASPELYQT GVTVAPITK* |
| 46 | MDIEKAGSRR EEEEPIVQRP RLDKGKGKAH VFAPPMNYNR IMDKHKQEKM SPAGWKRGVA |
| | IFDFVLRLIA AITAMAAAAK MATTEETLPF FTQFLQFQAD YTDLPTMSSF VIVNSIVGGY |
| | LTLSLPFSIV CILRPLAVPP RLFLILCDTV MMGLTLMAAS ASAAIVYLAR NGNSSSNWLP |
| | VCQQFGDFCQ GTSGAVVASF IAATLLMFLV ILSAFALKRT T* |
| 47 | MTTEEKEILA AKLEEQKIDL DKPEVEDDDD NEDDDSDDDD KDDDEADGLD GEAGGKSKQS |
| | RSEKKSRKAM LKLGMKPITG VSRVTVKKSK NILFVISKPD VFKSPASDTY VIFGEAKIED |
| | LSSQIQSQAA EQFKAPDLSN VISKGESSSA AVVQDDEEVD EEGVEPKDIE LVMTQAGVSR |
| | PNAVKALKAA DGDIVSAIME LTT* |
| 48 | MLPSDAADPS VCYVPNPYNP YQYYNVYGSG QEWTDYPAYT NPEGVDMNSG IYGENGTVVY |
| | PQGYGYAAYP YSPATSPAPQ LGGEGQLYGA QQYQYPNYFP NSGPYASSVA TPTQPDLSAN |
| | KPAGVKTLPA DSNNVASAAG ITKGSNGSAP VKPTNQATLN TSSNLYGMGA PGGGLAAGYQ |
| | DPRYAYEGYY APVPWHDGSK YSDVQRPVSG SGVASSYSKS STVPSSRNQN YRSNSHYTSV |
| | HQPSSVTGYG TAQGYYNRMY QNKLYGQYGS TGRSALGYGS SGYDSRTNGR GWAATDNKYR |
| | SWGRGNSYYY GNENNVDGLN ELNRGPRAKG TKNQKGNLDD SLEVKEQTGE SNVTEVGEAD |
| | NTCVVPDREQ YNKEDFPVDY ANAMFFIIKS YSEDDVHKSI KYNVWASTPN GNKKLAAAYQ |
| | EAQQKAGGCP IFLFFSVNAS GQFVGLAEMT GPVDFNTNVE YWQQDKWTGS FPLKWHIVKD |
| | VPNSLLKHIT LENNENKPVT NSRDTQEVKL EQGLKIVKIF KEHSSKTCIL DDFSFYEVRQ |
| | KTILEKKAKQ TQKQVSEEKV TDEKKESATA ESASKESPAA VQTSSDVKVA ENGSVAKPVT |
| | GDVVANGC* |
| 49 | MLAIFDKNVA KTPEALQGQE GGSVCALKDR FLPNHFSSVY PGAVTINLGS SGFIACSLEK |
| | QNPLLPRLFA VVDDMFCIFQ GHIENVPILK QQYGLTKTAT EVTIVIEAYR TLRDRGPYSA |
| | EQVVRDFQGK FGFMLYDCST QNVFLAGDVD GSVPLYWGTD AEGHLVVSDD VETVKKGCGK |
| | SFAPFPKGCF FTSSGGLRSY EHPSNELKPV PRVDSSGEVC GVTFKVDSEA KKEAMPRVGS |
| | VQNWSKQI* |
| 50 | MVNIPKTKNT YCKNKECKKH TLHKVTQYKK GKDSLAAQGK RRYDRKQSGY GGQTKPVFHK |
| | KAKTTKKIVL RLQCQSCKHF SQRPIKRCKH FEIGGDKKGK GTSLF* |
| 51 | MEKSNGLRVI LFPLPLQGCI NPMIQLAKIL HSRGFSITVI HTCFNAPKAS SHPLFTFLEI |
| | PDGLSETEKR TNNTKLLLTL LNRNCESPFR ECLSKLLQSA DSETGEEKQR ISCLIADSGW |
| | MFTQPIAQSL KLPILVLSVF TVSFFRCQFV LPKLRREVYL PLQDSEQEDL VQEFPPLRKK |
| | DIVRILDVET DILDPFLDKV LQMTKASSGL IFMSCEELDH DSVSQAREDF KIPIFGIGPS |
| | HSHFPATSSS LSTPDETCIP WLDKQEDKSV IYVSYGSIVT ISESDLIEIA WGLRNSDQPF |
| | LLVVRVGSVR GREWIETIPE EIMEKLNEKG KIVKWAPQQD VLKHRAIGGF LTHNGWSSTV |
| | ESVCEAVPMI CLPFRWDQML NARFVSDVWM VGINLEDRVE RNEIEGAIRR LLVEPEGEAI |
| | RERIEHLKEK VGRSFQQNGS AYQSLQNLID YISSF* |
| 52 | MADGEDIQPL VCDNGTGMVK AGFAGDDAPR AVFPSIVGRP RHTGVMVGMG QKDAYVGDEA |
| | QSKRGILTLK YPIEHGIVSN WDDMEKIWHH TFYNELRVAP EEHPVLLTEA PLNPKANREK |
| | MTQIMFETFN VPAMYVAIQA VLSLYASGRT TGIVLDSGDG VSHTVPIYEG YALPHAILRL |
| | DLAGRDLTDS LMKILTERGY MFTTTAEREI TRDIKEKLAY VALDYEQELE TAKSSSSVEK |
| | NYELPDGQVI TIGAERFRCP EVLFQPSLIG MEAPGIHETT YNSIMKCDVD IRKDLYGNIV |
| | LSGGSTMFPG IADRMSKEIT ALAPSSMKIK VVAPPERKYS VWIGGSILAS LSTFQQMWIS |
| | KSEYDESGPS IVHRKCF* |
| 53 | MEWEKWYLDA VLVPSALLMM FGYHIYLWYK VRTDPFCTIV GTNSRARRSW VAAIMKDNEK |
| | KNILAVQTLR NTIMGGTLMA TTCILLCAGL AAVLSSTYSI KKPLNDAVYG AHGDFTVALK |
| | YVTILTIFLF AFFSHSLSIR FINQVNILIN APQEPFSDDF GEIGSFVTPE YVSELLEKAF |
| | LLNTVGNRLF YMGLPLMLWI FGPVLVFLSS ALIIPVLYNL DFVFLLSNKE KGKVDCNGGC |
| | DDNFSP* |
| 54 | MGDARDNEAY EEELLDYEEE DEKVPDSGNK VNGEAVKKGY VGIHSSGFRD FLLKPELLRA |
| | IVDSGFEHPS EVQHECIPQA ILGMDVICQA KSGMGKTAVF VLSTLQQIEP SPGQVSALVL |
| | CETRELAYQI CNEFVRESTY LPDTKVSVFY GGVNIKIHKD LLKNECPHIV VGTPGRVLAL |
| | AREKDLSLKN VRHFILDECD KMLESLDMRR DVQEIFKMTP HDKQVMMFSA TLSKEIRPVC |
| | KKFMQDPMEI YVDDEAKLTL HGLVQHYIKL SEMEKTRKLN DLLDALDFNQ VVIFVKSVSR |
| | AAELNKLLVE CNFPSICIHS GMSQEERLTR YKSFKEGHKR ILVATDLVGR GIDIERVNIV |
| | INYDMPDSAD TYLHRVGRAG RFGTKGLAIT FVASASDSEV LNQVQERFEV DIKELPEQID |
| | TSTYSKCEIP YQLFV* |
| 55 | MATISNLANL PRATCVDSKS SSSSSVLPRS FVNFRALNAK LSSSQLSLRY NQRSIPSLSV |
| | RCSVSGGNGT AGKRTTLHDL YEKEGQSPWY DNLCRPVTDL LPLIARGVRG VTSNPAIFQK |
| | AISTSNAYND QFRTLVESGK DIESAYWELV VKDIQDACKL FEPIYDQTEG ADGYVSVEVS |
| | PRLADDTQGT VEAAKYLSKV VNRRNVYIKI PATAPCIPSI RDVIAAGISV NVTLIFSIAR |
| | YEAVIDAYLD GLEASGLDDL SRVTSVASFF VSRVDTLMDK MLEQIGTPEA LDLRGKAAVA |
| | QAALAYKLYQ QKFSGPRWEA LVKKGAKKQR LLWASTSVKN PAYSDTLYVA PLIGPDTVST |
| | MPDQALEAFA DHGIVKRTID ANVSEAEGIY SALEKLGIDW NKVGEQLEDE GVDSFKKSFE |
| | SLLGTLQDKA NTLKLASH* |
| 56 | MESPKNSLIP SFLYSSSSSP RSFLLDQVLN SNSNAAFEKS PSPAPRSSPT SMISRKNFLI |
| | ASPTEPGKGI EMYSPAFYAA CTFGGILSCG LTHMTVTPLD LVKCNMQIDP AKYKSISSGF |
| | GILLKEQGVK GFFRGWVPTL LGYSAQGACK FGFYEYFKKT YSDLAGPEYT AKYKTLIYLA |
| | GSASAEIIAD IALCPFEAVK VRVQTQPGFA RGMSDGFPKF IKSEGYGGLY KGLAPLWGRQ |
| | IPYTMMKFAS FETIVEMIYK YAIPNPKSEC SKGLQLGVSF AGGYVAGVFC AIVSHPADNL |
| | VSFLNNAKGA TVGDAVKKIG MVGLFTRGLP LRIVMIGTLT GAQWGLYDAF KVFVGLPTTG |
| | GVAPAPAIAA TEAKA* |
| 57 | MVEPANTVGL PVNPTPLLKD ELDIVIPTIR NLDFLEMWRP FLQPYHLIIV QDGDPSKKIH |
| | VPEGYDYELY NRNDINRILG PKASCISFKD SACRCFGYMV SKKKYIFTID DDCFVAKDPS |
| | GKAVNALEQH IKNLLCPSSP FFFNTLYDPY REGADFVRGY PFSLREGVST AVSHGLWLNI |
| | PDYDAPTQLV KPKERNTRYV DAVMTIPKGT LFPMCGMNLA FDRDLIGPAM YFGLMGDGQP |
| | IGRYDDMWAG WCIKVICDHL SLGVKTGLPY IYHSKASNPF VNLKKEYKGI FWQEEIIPFF |
| | QNAKLSKEAV TVQQCYIELS KMVKEKLSSL DPYFDKLADA MVTWIEAWDE LNPPAASGKA |
| | * |
| 58 | MSETNKNAFQ AGQAAGKAEE KSNVLLDKAK DAAAAAGASA QQAGKSISDA AVGGVNFVKD |
| | KTGLNK* |
| 59 | MASHIVGYPR MGPKRELKFA LESFWDGKST AEDLQKVSAD LRSSIWKQMS AAGTKFIPSN |
| | TFAHYDQVLD TTAMLGAVPP RYGYTGGEIG LDVYFSMARG NASVPAMEMT KWFDTNYHYI |
| | VPELGPEVNF SYASHKAVNE YKEAKALGVD TVPVLVGPVS YLLLSKAAKG VDKSFELLSL |
| | LPKILPIYKE VITELKAAGA TWIQLDEPVL VMDLEGQKLQ AFTGAYAELE STLSGLNVLV |
| | ETYFADIPAE AYKTLTSLKG VTAFGFDLVR GTKTLDLVKA GFPEGKYLFA GVVDGRNIWA |
| | NDFAASLSTL QALEGIVGKD KLVVSTSCSL LHTAVDLINE TKLDDEIKSW LAFAAQKVVE |
| | VNALAKALAG QKDEALFSAN AAALASRRSS PRVTNEGVQK AAAALKGSDH RRATNVSARL |
| | DAQQKKLNLP ILPTTTTGSF PQTVELRRVR REYKAKKVSE EDYVKAIKEE IKKVVDLQEE |
| | LDIDVLVHGE PERNDMVEYF GEQLSGFAFT ANGWVQSYGS RCVKPPVIYG DVSRPKAMTV |
| | FWSAMAQSMT SRPMKGMLTG PVTILNWSFV RNDQPRHETC YQIALAIKDE VEDLEKGGIG |
| | VIQIDEAALR EGLPLRKSEH AFYLDWAVHS FRITNCGVQD STQIHTHMCY SHFNDIIHSI |
| | IDMDADVITI ENSRSDEKLL SVFREGVKYG AGIGPGVYDI HSPRIPSSEE IADRVNKMLA |
| | VLEQNILWVN PDCGLKTRKY TEVKPALKNM VDAAKLIRSQ LASAK* |
| 60 | MEVLDRRDDE IRDSGNMDSI KSHYVTDSVS EERRSRELKD GDHPLRYKFS IWYTRRTPGV |
| | RNQSYEDNIK KMVEFSTVEG FWACYCHLAR SSLLPSPTDL HFFKDGIRPL WEDGANCNGG |
| | KWIIRFSKVV SARFWEDLLL ALVGDQLDDA DNICGAVLSV RFNEDIISVW NRNASDHQAV |
| | MGLRDSIKRH LKLPHAYVME YKPHDASLRD NSSYRNTWLR G* |
| 61 | GATCTCTGTTTCACAAG |
| 62 | GATCTGTGTTGTTAATT |
| 63 | GATCCTTGCTTGAGCTA |
| 64 | GATCCGTAACTCTTGAA |
| 65 | GATCCCTCTTTACAGTT |
| 66 | GATCCCGTGCTGCAGCT |
| 67 | GATCACTGGAATTTGAG |
| 68 | GATCGTTCCCTTGCTGC |
| 69 | GATCTTTTTTTTGTTCA |
| 70 | GATCCAATCTTAAAGGT |
| 71 | GATCATTTATGAGAAGC |
| 72 | GATCAATCAAGGAGAGT |
| 73 | GATCAGCATTTACAGTG |
| 74 | GATCCTCTTGATTAAAT |
| 75 | GATCTCAAAGGGTGAGT |
| 76 | GATCCGTTTCTTTGCCC |
| 77 | GATCAAAACACAATCCT |
| 78 | GATCGGTGGTGACAAGA |
| 79 | GATCGTTTCAACAAAAC |
| 80 | GATCAATCCTTGCATCC |
| 81 | GATCTTTGGGCCTGTGC |
| 82 | GATCTATTATCAATTTA |
| 83 | GATCATGGAATAGTGAA |
| 84 | GATCGGGACGTTGACTG |
| 85 | GATCATTCCGTTCTTCC |
| 86 | GATCCGAGTCAACTTTG |
| 87 | GATCCACACTCACATGT |
| 88 | GATCAAAACTATAAGGA |
| 89 | GATCTGAAAGAGAGAAG |
| 90 | GATCATCTTTTTTCTCC |
| 91 | GATCATGCATATTTGTT |
| 92 | GATCATTGAGAATCCAG |
| 93 | GATCATTCAAATCTTGT |
| 94 | GATCTCGACTTCTCTGC |
| 95 | GATCGTCTTCAAGGGCA |
| 96 | GATCACACCTCTGAGTC |
| 97 | GATCTACTATTATTAAG |
| 98 | GATCCGTTGATTTGCTC |
| 99 | GATCCAGACAACATGAA |
| 100 | GATCCCAATTCCTTGTT |
| 101 | GATCTCTCTGTCTCCCA |
| 102 | GATCTCTATTGGCAATA |
| 103 | GATCTCTACTCTCTTCT |
| 104 | GATCTGAGATAGAGACA |
| 105 | GATCCATTGAGATAATT |
| 106 | GATCTATTCCAGCGGAA |
| 107 | GATCCTAGAATATTTTT |
| 108 | GATCCTGTCATGGAATA |
| 109 | GATCGTTCGTGGTACTT |
| 110 | GATCGGCTTCTGCTCGA |
| 111 | GATCGGCATTACGACCC |
| 112 | GATCTCCTTTTGATTCT |
| 113 | GATCAAAATTCTCAACC |
| 114 | GATCTTGCCTTTTAAAC |
| 115 | GATCTTGTATAATGACA |
| 116 | GATCTTTATGGTGCTAG |
| 117 | GATCAACCCGATTCTTG |
| 118 | GATCAAGATTTTTTTTA |
| 119 | GATCACGCCTTTGTTTC |
| 120 | GATCAAGAATGTGTATG |
| 121 | GATCTGATTTTCTCAAC |
| 122 | GATCACACCGCAATGCT |
| 123 | GATCGACTCTTCTCGTT |
| 124 | GATCAATATGGTTTTGA |
| 125 | GATCGCGTCTGAATTGT |
| 126 | GATCTCTGTCATAGACT |
| 127 | GATCTCGGCATGTGTGT |
| 128 | GATCTTGGGTGCAATTT |
| 129 | GATCAACATGAATGAGG |
| 130 | GATCTTCTGCTAGGGAT |
| 131 | GATCCCGTATCTTGAAC |
| 132 | GATCCAGAAATTTCCAA |
| 133 | GATCGCGTCGTGTTACT |
| 134 | GATCTTAGCTTATGACT |
| 135 | GATCTATATTTTTCTAA |
| 136 | GATCCTTTTTGTAGTTT |
| 137 | GATCGACGATGTCATCT |
| 138 | GATCATTGAGTATGTTT |
| 139 | GATCAATCAATGGTTCA |
| 140 | GATCGACTCTCTTACTT |
| 141 | GATCTTTGTTTTTAAGA |
| 142 | GATCTTGGTTTTTAGAG |
| 143 | GATCTATTCGGTGAAAA |
| 144 | GATCACAGTGAACCCCG |
| 145 | GATCTTGTGGACATCTC |
| 146 | GATCGTTAATTCAATGC |
| 147 | GATCGAAGAAGCAGACC |
| 148 | GATCTGTGTGTCGTCCA |
| 149 | GATCTTCTGTGCTATGT |
| 150 | GATCTCTGGATTCATCG |
| 151 | GATCAGATGCAATTTGC |
| 152 | GATCCTCTCCTATGATG |
| 153 | GATCTTTGTAACGCACC |
| 154 | GATCTCATAAATGTTGG |
| 155 | GATCTCTGTGAGATTTG |
| 156 | GATCTGTAGCAAACACA |
| 157 | GATCATGCCTCTGTTCA |
| 158 | GATCTGGCGGAGCACCA |
| 159 | GATCTGACAAACGCAAC |
| 160 | GATCAATCAACCTTATG |
| 161 | GATCTGTAAAATACTAC |
| 162 | GATCATAAAGAGACAGA |
| 163 | GATCCGTGGTGTTAAGA |
| 164 | GATCCTTAACTTGAGGA |
| 165 | GATCGCAGTCGAGGAAT |
| 166 | GATCTTCTTGTTCGCAT |
| 167 | GATCATTCTTCTTTTGG |
| 168 | GATCTCGTCTTTGTTTT |
| 169 | GATCAGATAAAACACCT |
| 170 | GATCTGTAGCCAATGGA |
| 171 | GATCCAAATCCAAAGAG |
| 172 | GATCAGAGGAGAACGTG |
| 173 | GATCTAAGCTTAGCATC |
| 174 | GATCACAGTTTTGAAAT |
| 175 | GATCCAGAGGCGTTCAA |
| 176 | GATCTGATGAGCCAAAG |
| 177 | GATCAAAGCCATTGAAG |
| 178 | GATCCCGTGAGTGGATG |
| 179 | GATCCTGTTTTTGATTG |
| 180 | GATCTGAATAGCTGCGC |
| 181 | GATCATATACCAGTATT |
| 182 | GATCACATCTTTACCAG |
| 183 | GATCCTTCTAAGACTAA |
| 184 | GATCATTTCTGTTAGAA |
| 185 | GATCGTGGCCGTTGGAT |
| 186 | GATCATGCTCTCCAAAC |
| 187 | GATCCCAAACCGATGGT |
| 188 | GATCATTAGTCTCTCAT |
| 189 | GATCGGTGTGTTATACA |
| 190 | GATCTTGTCTCTGAGTA |
| 191 | GATCTTTCGCCTCTTCT |
| 192 | GATCTGCTGAAACTGAA |
| 193 | GATCTTTTTTTTTGTGT |
| 194 | GATCTCATCCATCTTCT |
| 195 | GATCTAAATCTGTGAAA |
| 196 | GATCAAAAAAAAAAAAA |
| 197 | GATCAAAACAACCTGCG |
| 198 | GATCAAAACAATGAGGG |
| 199 | GATCAAAACTGTTACAC |
| 200 | GATCAAAAGCTCTTACA |
| 201 | GATCAAAATTTGAGGGG |
| 202 | GATCAAAATTTGTAGTG |
| 203 | GATCAAACTGGTGAAGG |
| 204 | GATCAAACTTTGCTTGC |
| 205 | GATCAAATCATCTTCCA |
| 206 | GATCAAATGTCCCCACC |
| 207 | GATCAACGCAGCCAAGG |
| 208 | GATCAACTCTTTACATG |
| 209 | GATCAACTGTCAATTCA |
| 210 | GATCAACTTAAGCAAAA |
| 211 | GATCAACTTATAAGTGC |
| 212 | GATCAAGAAAGAAGAAG |
| 213 | GATCAAGAAGGTAACGC |
| 214 | GATCAAGCTGTCTTCAA |
| 215 | GATCAAGTTTACAGGAT |
| 216 | GATCAATAATTGTTTCT |
| 217 | GATCAATCTAGCGAACA |
| 218 | GATCAATTGATGGCGCA |
| 219 | GATCACAGATTCTGAAT |
| 220 | GATCACAGCAAGAGTGG |
| 221 | GATCACATGAGGAAGAT |
| 222 | GATCACCTTGTTGCTGC |
| 223 | GATCACGACCAAGTCAT |
| 224 | GATCACGGTTCTCGTCG |
| 225 | GATCACTGCTTTGGCTC |
| 226 | GATCACTTTCAGTGATA |
| 227 | GATCACTTTTAACTGTT |
| 228 | GATCACTTTTTTGTGGG |
| 229 | GATCAGAAGAGCAACGT |
| 230 | GATCAGAAGCAGTGCGT |
| 231 | GATCAGAAGGAACTGCA |
| 232 | GATCAGAATCATCAATA |
| 233 | GATCAGATGCAATGTGT |
| 234 | GATCAGATGGGATGGTA |
| 235 | GATCAGATTTTCTTGGG |
| 236 | GATCAGCGCCACTCTTC |
| 237 | GATCAGTTAGCTTCTCT |
| 238 | GATCAGTTGATGCTGGA |
| 239 | GATCATATGTTGCTGGA |
| 240 | GATCATCAAAACCATCC |
| 241 | GATCATCAAAATCAGTC |
| 242 | GATCATCACTATTTCAT |
| 243 | GATCATCCCCTGTCTGT |
| 244 | GATCATCCTTCTTTGCC |
| 245 | GATCATCGTTTCGTGTA |
| 246 | GATCATCTATTGGATGA |
| 247 | GATCATCTCACCTTTGT |
| 248 | GATCATCTGAAACCATC |
| 249 | GATCATCTGTGAATTTT |
| 250 | GATCATCTTTTGAATGT |
| 251 | GATCATGAAATGGTATG |
| 252 | GATCATGATTTCCTTCT |
| 253 | GATCATGCAATCAAGCA |
| 254 | GATCATGTGTTTGGTTT |
| 255 | GATCATTCTCCTCGCAA |
| 256 | GATCATTGGGAAATGAT |
| 257 | GATCATTGTTGTCTCAC |
| 258 | GATCATTTTATGTGATT |
| 259 | GATCATTTTCCAAACGC |
| 260 | GATCATTTTGATGCTTT |
| 261 | GATCATTTTTCTCTAAT |
| 262 | GATCATTTTTTTTTTTT |
| 263 | GATCCAAAAGACAAACA |
| 264 | GATCCAAAGAGTTGGAG |
| 265 | GATCCAAATCAACCTAA |
| 266 | GATCCAAGCTTTTAATG |
| 267 | GATCCAATAATACATAC |
| 268 | GATCCAATGGCACCAGC |
| 269 | GATCCAATTTGGTCAGA |
| 270 | GATCCACATGGAGGTAG |
| 271 | GATCCACCTGATGATGT |
| 272 | GATCCACGAGTTTCAGG |
| 273 | GATCCACGCGTGGGAGA |
| 274 | GATCCAGAAGCCGGAGT |
| 275 | GATCCAGAAGTTCTTGC |
| 276 | GATCCAGAGGTCTGGTT |
| 277 | GATCCAGCAGTGGTGTT |
| 278 | GATCCAGTTATTATGGA |
| 279 | GATCCAGTTTTTGTTTG |
| 280 | GATCCATGAACTGGACC |
| 281 | GATCCATTCACTGTTAA |
| 282 | GATCCATTCCGCAGTTC |
| 283 | GATCCATTTGTGATGAA |
| 284 | GATCCCAAACGACAAAA |
| 285 | GATCCCAAATTCCCAAT |
| 286 | GATCCCAGATTACGATT |
| 287 | GATCCCATTATCGCTAA |
| 288 | GATCCCATTTCTCACTG |
| 289 | GATCCCGATTGGAGTGC |
| 290 | GATCCCTCCGAAGCAGT |
| 291 | GATCCCTGCATACGGTG |
| 292 | GATCCGCTTCGCCTTCA |
| 293 | GATCCGGATATTTACAC |
| 294 | GATCCGTATCGTCGATT |
| 295 | GATCCGTCCTACTTGTC |
| 296 | GATCCGTCTTATTGCGT |
| 297 | GATCCTAACCATTATCC |
| 298 | GATCCTAGGAGAATACA |
| 299 | GATCCTATTCGTTGTTG |
| 300 | GATCCTCATCTTTCCTA |
| 301 | GATCCTCCTCGGACGAA |
| 302 | GATCCTCGGATGTGGCA |
| 303 | GATCCTGACGCCGTAGC |
| 304 | GATCCTGAGAATTTCTT |
| 305 | GATCCTTATCATCCGAG |
| 306 | GATCCTTATTTGGTGCC |
| 307 | GATCCTTCCGCAATGTT |
| 308 | GATCCTTCGTTAACGGC |
| 309 | GATCCTTGGATTTGGTC |
| 310 | GATCCTTGTGGCGACTG |
| 311 | GATCCTTTAGAACATTT |
| 312 | GATCCTTTCGACAAGAT |
| 313 | GATCCTTTCTTGGAAGA |
| 314 | GATCCTTTCTTTGGGGT |
| 315 | GATCCTTTTATCGAATC |
| 316 | GATCGAACCAAGTTTCA |
| 317 | GATCGAACCAGAGATAT |
| 318 | GATCGAATTCCTGGAAG |
| 319 | GATCGACAGTCTGGAGA |
| 320 | GATCGACGACTGGACTC |
| 321 | GATCGATGCCCTTGTGA |
| 322 | GATCGCCATTGAGAACA |
| 323 | GATCGCTGCAACGATGA |
| 324 | GATCGCTGCTCAGTTTG |
| 325 | GATCGGAAAGATTGTGG |
| 326 | GATCGGAATTCGTGATG |
| 327 | GATCGGAATTTCATGTG |
| 328 | GATCGGATTTTTTCTGA |
| 329 | GATCGGGAAGAGAGGAG |
| 330 | GATCGTATACTTCGTCC |
| 331 | GATCGTCAAGAAGAAGC |
| 332 | GATCGTCGTTCGATGAT |
| 333 | GATCGTGGTGTCCTCGC |
| 334 | GATCGTTAATTTTTTTT |
| 335 | GATCTAAACTTTTATGC |
| 336 | GATCTAAGTGGAATCTT |
| 337 | GATCTAATAGCAGAGTT |
| 338 | GATCTACCCGATTCTTT |
| 339 | GATCTACGCGTCCCTCT |
| 340 | GATCTACGTAAGTTTTC |
| 341 | GATCTACTCAACGAAGC |
| 342 | GATCTAGGCGCTTTTAC |
| 343 | GATCTATCCAGTTTGGT |
| 344 | GATCTATCTATTATTCC |
| 345 | GATCTATTCATAGAAGT |
| 346 | GATCTATTCTGTCCAAG |
| 347 | GATCTCAAAGTGACTGT |
| 348 | GATCTCAAGTTTCAATC |
| 349 | GATCTCAGATATTTTAA |
| 350 | GATCTCATACATTATGT |
| 351 | GATCTCATTATGCAATT |
| 352 | GATCTCCAGTTCGATAT |
| 353 | GATCTCCGTCCCAAGAA |
| 354 | GATCTCGAAAGCTATCA |
| 355 | GATCTCGGTGTTCCTTC |
| 356 | GATCTCTACAATTAGTG |
| 357 | GATCTCTCTAGCCTTTG |
| 358 | GATCTCTCTCGGCCTTG |
| 359 | GATCTCTCTTTATTGTC |
| 360 | GATCTCTTACACGTGCC |
| 361 | GATCTCTTTATGAAAGA |
| 362 | GATCTCTTTGTGACTAT |
| 363 | GATCTCTTTCTTTTTCT |
| 364 | GATCTGAAATCCGCCGT |
| 365 | GATCTGACTAATGTCAT |
| 366 | GATCTGAGTTTTATTTT |
| 367 | GATCTGATTGGTTTTGG |
| 368 | GATCTGATTGTGTTACC |
| 369 | GATCTGCACAAAGCATG |
| 370 | GATCTGCCAAAAGCACC |
| 371 | GATCTGCTGAAGAAAGT |
| 372 | GATCTGCTGGGAAAGTC |
| 373 | GATCTGGACCTTGTCCC |
| 374 | GATCTGGAGGTGCCTAA |
| 375 | GATCTGGTCTACTATAT |
| 376 | GATCTGGTTCGTTCCGT |
| 377 | GATCTGTTCTTCCAGCA |
| 378 | GATCTGTTTCATTAGAC |
| 379 | GATCTTAGTGACGATGA |
| 380 | GATCTTATTGTTGGTGA |
| 381 | GATCTTCAGTCTTGAGT |
| 382 | GATCTTCCCTTTTCTTT |
| 383 | GATCTTCTTGAGGAGGA |
| 384 | GATCTTCTTGGCATGCA |
| 385 | GATCTTGCAGCATTGGA |
| 386 | GATCTTGCTCGGCTTGC |
| 387 | GATCTTGTACCTTCTGA |
| 388 | GATCTTGTTGAAGGATG |
| 389 | GATCTTGTTTCTCGGTC |
| 390 | GATCTTTATCTTTATCT |
| 391 | GATCTTTCTTGTTTTGT |
| 392 | GATCTTTGTTGGTGTAA |
| 393 | GATCTTTTCTTGGATGA |
| 394 | GATCTTTTGGTCTTTTT |
| 395 | GATCTTTTTGGGGATAA |
| 396 | GATCTTTTTGTATGTTG |
| 397 | GATCTGAAAGAGAGAAG |
| 398 | GATCATCTTTTTTCTCC |
| 399 | GATCACTGGAATTTGAG |
| 400 | GATCGTTCCCTTGCTGC |
| 401 | GATCCAATCTTAAAGGT |
| 402 | GATCAATCAAGGAGAGT |
| 403 | GATCATGCATATTTGTT |

What is claimed is:

1. A composition comprising at least one expression vector, wherein the at least one expression vector comprises a nucleic acid comprising:
(a) at least one polynucleotide sequence selected from the group consisting of: SEQ ID NO: 1-SEQ ID NO: 30 or a sequence complementary thereto;
(b) at least one polynucleotide sequence comprising a conservative variation of a polynucleotide sequence of (a);
(c) at least one polynucleotide encoding a polypeptide sequence selected from the group consisting of SEQ ID NO: 31-SEQ ID NO: 60, or conservative variations thereof;
(d) at least one polynucleotide sequence that hybridizes under stringent conditions to a polynucleotide sequence of (a) or (b);
(e) at least one polynucleotide that is at least about 70% identical to a polynucleotide sequence of (a), or (b); or,
(f) at least one polynucleotide sequence comprising at least 10 contiguous nucleotides of a polynucleotide sequence selected from the group consisting of: SEQ ID NO: 1-SEQ ID NO: 30, or a sequence complementary thereto.

2. The composition of claim 1, wherein the at least one expression vector comprises a promoter operably linked to the nucleic acid comprising the polynucleotide of (a), (b), (c), (d) or (e).

3. The composition of claim 1, wherein the nucleic acid encodes a polypeptide.

4. The composition of claim 1, wherein the polypeptide comprises a polypeptide subsequence of SEQ ID NO: 31-SEQ ID NO: 60.

5. The composition of claim 1, wherein the nucleic acid encodes a sense or antisense RNA.

6. A cell comprising the at least one expression vector of claim 1.

7. The cell of claim 6, which cell expresses a polypeptide selected from the group consisting of SEQ ID NO: 31-SEQ ID NO: 60, and conservative variations thereof.

8. An isolated or recombinant polypeptide comprising:
(a) an amino acid sequence selected from the group consisting of SEQ ID NO: 31-SEQ ID NO: 60, and conservative variants thereof;
(b) an amino acid sequence encoded by a polynucleotide sequence selected from the group consisting of SEQ ID NO: 1 to SEQ ID NO: 30, and conservative variations thereof;
(c) an amino acid sequence encoded by a polynucleotide sequence that hybridizes under stringent conditions to a polynucleotide sequence selected from the group consisting of SEQ ID NO: 1 to SEQ ID NO: 30;
(d) an amino acid sequence encoded by a polynucleotide sequence that is at least about 70% identical to a polynucleotide selected from the group consisting of SEQ ID NO: 1 to SEQ ID NO: 30, or
(e) a polypeptide comprising an amino acid subsequence of (a), (b), (c) or (d).

9. The isolated or recombinant polypeptide of claim 8, comprising a fusion protein.

10. The isolated or recombinant polypeptide of claim 8, comprising a peptide or polypeptide tag.

11. The isolated or recombinant polypeptide of claim 10, wherein the peptide or polypeptide tag comprises a reporter peptide or polypeptide.

12. The isolated or recombinant polypeptide of claim 10, wherein the peptide or polypeptide tag comprises an epitope.

13. The isolated or recombinant polypeptide of claim 10, wherein the peptide or polypeptide tag comprises a localization signal or sequence.

14. An array of polypeptides comprising two or more different polypeptides of claim 8.

15. An antibody specific for the isolated or recombinant polypeptide of claim 8.

16. The antibody of claim 15, wherein the antibody comprises a monoclonal antibody or polyclonal serum.

17. The antibody of claim 15, which antibody is specific for an epitope comprising a subsequence of a polypeptide selected from the group consisting of SEQ ID NO: 31-SEQ ID NO: 60.

18. An isolated or recombinant polypeptide which specifically binds to the antibody of claim 15.

19. A cell comprising at least one exogenous nucleic acid, which cell expresses a polypeptide of claim 8.

20. The cell of claim 19, wherein the expressed polypeptide is encoded by the exogenous nucleic acid.

21. The cell of claim 19, wherein the exogenous nucleic acid comprises a promoter, which promoter regulates transcription of an endogenous nucleic acid encoding the polypeptide.

22. A labeled probe comprising a nucleic acid or polypeptide comprising:
(a) a polynucleotide sequence selected from the group consisting of: SEQ ID NO: 1-SEQ ID NO: 30; conservative variants of any one of SEQ ID NO: 1-SEQ ID NO: 30; or, a subsequence of SEQ ID NO: 1-SEQ ID NO: 30; or a conservative variant thereof comprising at least 10 nucleotides; or a complementary sequence thereof;
(b) a polypeptide or peptide comprising an amino acid sequence selected from the group consisting of: SEQ ID NO: 31-SEQ ID NO: 60; a conservative variant of any one of SEQ ID NO: 31-SEQ ID NO: 60, or, a subsequence of one or more of SEQ ID NO: 31-SEQ ID NO: 60, or one or more conservative variants thereof, comprising at least six amino acids; or,
(c) an antibody specific for a polypeptide or peptide sequence of (b).

23. The labeled probe of claim 22, wherein the polynucleotide sequence comprises a subsequence of SEQ ID NO: 1-SEQ ID NO: 30, comprising at least 12 nucleotides.

24. The labeled probe of claim 22, wherein the polynucleotide sequence comprises a subsequence of SEQ ID NO: 1-SEQ ID NO: 30, comprising at least 14 nucleotides.

25. The labeled probe of claim 22, wherein the polynucleotide sequence comprises a subsequence of SEQ ID NO: 1-SEQ ID NO: 30, comprising at least 16 nucleotides.

26. The labeled probe of claim 22, wherein the polynucleotide sequence comprises subsequence of SEQ ID NO: 1-SEQ ID NO: 30 comprising at least 17 nucleotides.

27. The labeled probe of claim 22, comprising an antigenic peptide.

28. The labeled probe of claim 22, comprising a fusion protein.

29. The labeled probe of claim 22, comprising an epitope tag.

30. The labeled probe of claim 22, comprising an isotopic, fluorescent, fluorogenic or colorimetric label.

31. The labeled probe of claim 22, comprising a DNA or RNA molecule.

32. A labeled probe of claim 22, comprising a cDNA, an amplification product, a transcript, a restriction fragment, or an oligonucleotide.

33. A labeled probe of claim 22, comprising an oligonucleotide consisting of a polynucleotide sequence selected from a subsequence of SEQ ID NO: 61 to SEQ ID NO: 403, or a conservative variation thereof.

34. A marker set for predicting at least one growth trait of a plant cell, the marker set comprising a plurality of members, which members comprise:
(a) one or more polynucleotides sequences selected from the group consisting of: SEQ ID NO: 1-SEQ ID NO: 30 or SEQ ID NO: 61-SEQ ID NO: 403; a conservative variant of any one of SEQ ID NO: 1-SEQ ID NO: 30 or SEQ ID NO: 61-SEQ ID NO: 403; a subsequence of SEQ ID NO: 1-SEQ ID NO: 30, SEQ ID NO: 61-SEQ ID NO: 403, or a conservative variant thereof comprising at least 10 nucleotides; and, a complementary sequence thereof;
(b) one or more polypeptides or peptides comprising an amino acid selected from the group consisting of: SEQ ID NO: 31 to SEQ ID NO: 60; a conservative variant of any one of SEQ ID NO: 31 to SEQ ID NO: 60; or a subsequence of SEQ ID NO: 31-SEQ ID NO: 60 or a conservative variant thereof comprising at least six amino acids; and/or,
(c) one or more antibodies specific for a polypeptide or peptide sequence of (b).

35. The marker set of claim 34, wherein the nucleic acids comprise oligonucleotides, expression products, or amplification products.

36. The marker set of claim 35, wherein the oligonucleotides are synthetic oligonucleotides.

37. The marker set of claim 34, comprising a plurality of labeled nucleic acid probes.

38. The marker set of claim 34, comprising a plurality of polypeptides or peptides.

39. The marker set of claim 34, comprising a plurality of antibodies.

40. The marker set of claim 34, comprising a plurality of members, which members include nucleic acids and polypeptides.

41. The marker set of claim 34, wherein the nucleic acids or polypeptides are logically or physically arrayed.

42. The marker set of claim 34, wherein the nucleic acids or polypeptides are physically arrayed in a solid phase or liquid phase array.

43. The marker set of claim 41, wherein the array comprises a bead array.

44. The marker set of claim 34, wherein each member of the marker set comprises at least 10 contiguous nucleotides from at least one of SEQ ID NO: 1-SEQ ID NO: 30.

45. The marker set of claim 34, comprising a plurality of members that together comprise a plurality of sequences or subsequences selected from a plurality of nucleic acids represented by SEQ ID NO: 61-SEQ ID NO: 403.

46. The marker set of claim 34, comprising a majority of members that together comprise a majority of sequences or subsequences selected from a majority of nucleic acids represented by SEQ ID NO: 61-SEQ ID NO: 403.

47. The marker set of claim 34, wherein each member of the marker set comprises at least 10 contiguous nucleotides from at least one of SEQ ID NO: 61-SEQ ID NO: 403.

48. The marker set of claim 34, wherein each member of the marker set comprises at least six contiguous amino acids from at least one of SEQ ID NO: 31-SEQ ID NO: 60.

49. The marker set of claim 34, comprising at least one antibody specific for each of SEQ ID NO: 31-SEQ ID NO: 60, or a subsequence thereof.

50. The marker set of claim 34, wherein a plant growth trait is predicted by hybridizing the nucleic acids of the marker set to a DNA or RNA sample from a cell or tissue, and detecting at least one polymorphic polynucleotide or differentially expressed expression product.

51. An array comprising the marker set of claim 34.

52. A method for modulating a plant growth trait, the method comprising:
modulating expression or activity of at least one polypeptide encoded by a nucleic acid comprising:
(a) at least one polynucleotide sequence selected from the group consisting of: SEQ ID NO: 1-SEQ ID NO: 30 or a sequence complementary thereto;
(b) at least one polynucleotide sequence comprising a conservative variation of a polynucleotide sequence of (a);
(c) at least one polynucleotide encoding a polypeptide sequence selected from the group consisting of SEQ ID NO: 31-SEQ ID NO: 60, or conservative variations thereof;
(d) at least one polynucleotide sequence that hybridizes under stringent conditions to a polynucleotide sequence of (a) or (b);
(e) at least one polynucleotide that is at least about 70% identical to a polynucleotide sequence of (a), or (b); or,
(f) at least one polynucleotide sequence comprising at least 10 contiguous nucleotides of a polynucleotide sequence selected from the group consisting of: SEQ ID NO: 1-SEQ ID NO: 30, or a sequence complementary thereto.

53. The method of claim 52, comprising modulating expression or activity of at least one polypeptide contributing to a plant growth trait.

54. The method of claim 52, comprising modulating a plant growth trait in a flowering plant.

55. The method of claim 52, comprising modulating a plant growth trait in a member of the family Brassicaceae.

56. The method of claim 52, comprising modulating a plant growth trait in a plant selected from the group consisting of Arabidopsis, Brassica, Zea, Oryza, Triticum, Hordeum, Lolium, Sorghum, Glycine, Medicago, Helianthus, Lactuca, Beta, Vitis, Solanum, Lycopersicon, Capsicum, Gossypium, Hevea, Linum, Prunus, Citrus, Populus, Pinus, Quercus, and Saccharomyces.

57. The method of claim 52, comprising modulating expression by expressing an exogenous nucleic acid comprising a polynucleotide sequence selected from SEQ ID NO: 1 to SEQ ID NO: 30.

58. The method of claim 57, comprising modulating expression by inducing or suppressing expression of an endogenous nucleic acid.

59. The method of claim 58, wherein the endogenous nucleic acid encodes a polypeptide selected from among SEQ ID NO: 31-SEQ ID NO: 60, or homologues thereof.

60. The method of claim 57, comprising introducing the exogenous nucleic acid comprising at least one promoter, which promoter regulates expression of an endogenous nucleic acid modulating a plant growth trait.

61. The method of claim 57, further comprising detecting altered expression or activity of an expression product encoded by a nucleic acid comprising a polynucleotide sequence selected from SEQ ID NO: 1-SEQ ID NO: 30, or conservative variants thereof.

62. The method of claim 61, comprising detecting altered expression or activity in a high throughput assay.

63. The method of claim 52, wherein expression is modulated in response to an environmental factor, a chemical or biological agent, a pathogen, a bacteria, a virus, a fungus, or an insect.

64. The method of claim 63, comprising detecting altered expression or activity in response to the presence of a fertilizer, or an herbicide.

65. The method of claim 63, wherein a plurality of expression products are detected.

66. The method of claim 65, wherein the plurality of expression products are detected in an array.

67. The method of claim 66, wherein the array comprises a bead array.

68. The method of claim 63, wherein a data record comprising the altered expression or activity is recorded in a database.

69. The method of claim 68, wherein the database comprises a plurality of character strings recorded on a computer or in a computer readable medium.

70. A method for detecting genes for a plant growth trait, the method comprising:
(i) providing a subject cell or tissue sample of nucleic acids;
(ii) detecting at least one polymorphic nucleic acid or at least one expression product corresponding to a polynucleotide sequence, comprising;
(a) at least one polynucleotide sequence selected from the group consisting of: SEQ ID NO: 1-SEQ ID NO: 30 or a sequence complementary thereto;
(b) at least one polynucleotide sequence comprising a conservative variation of a polynucleotide sequence of (a);
(c) at least one polynucleotide encoding a polypeptide sequence selected from the group consisting of SEQ ID NO: 31-SEQ ID NO: 60, or conservative variations thereof;
(d) at least one polynucleotide sequence that hybridizes under stringent conditions to a polynucleotide sequence of (a) or (b);
(e) at least one polynucleotide that is about 70% identical to a polynucleotide sequence of (a), or (b); or,
(f) at least one polynucleotide sequence comprising at least 10 contiguous nucleotides of a polynucleotide sequence selected from the group consisting of: SEQ ID NO: 1-SEQ ID NO: 30, or a sequence complementary thereto.

71. The method of claim 70, wherein the expression product comprises an RNA.

72. The method of claim 70, wherein the detecting step comprises qualitative detection.

73. The method of claim 70, wherein the detecting step comprises quantitative detection.
